(19) United States
(12) Patent Application Publication
(10) Pub. No.: US 2022/0304992 A1
(43) Pub. Date: Sep. 29, 2022

(54) METHODS OF REJUVENATING AGED TISSUE BY INHIBITING 15-HYDROXYPROSTAGLANDIN DEHYDROGENASE (15-PGDH)

(71) Applicant: The Board of Trustees of the Leland Stanford Junior University, Stanford, CA (US)

(72) Inventors: Helen M. Blau, Stanford, CA (US); Adelaida Rosa Palla, Stanford, CA (US); Andrew Tri Van Ho, Paris (FR)

(73) Assignee: The Board of Trustees of the Leland Stanford Junior University, Stanford, CA (US)

(21) Appl. No.: 17/616,966

(22) PCT Filed: Jun. 11, 2020
(86) PCT No.: PCT/US2020/037207

§ 371 Date: Dec. 6, 2021

Related U.S. Application Data
(60) Provisional application No. 62/860,180, filed on Jun. 11, 2019.
(60) Provisional application No. 62/875,915, filed on Jul. 18, 2019.
(60) Provisional application No. 62/882,981, filed on Aug. 5, 2019.
(60) Provisional application No. 62/883,025, filed on Aug. 5, 2019.

(51) Int. Cl. A61K 31/4365; A61P 21/00

(52) U.S. Cl. CPC A61K 31/4365; A61P 21/00

(57) ABSTRACT

The present disclosure provides compositions and methods based on the identification of 15-hydroxyprostaglandin dehydrogenase (15-PGDH) as a therapeutic target in aging, dystrophic muscle to improve muscle atrophy, increase muscle mass, function and strength. Further provided herein are compositions and methods for the rejuvenation of aged tissue. In particular, 15-PGDH inhibitors, such as SW033291, are used to elevate the levels of prostaglandin E2 (PGE2) in the muscle or tissue.

### CROSS-REFERENCE

This application claims the benefit of U.S. Provisional Patent Application No. 62/860,180, filed Jun. 11, 2019; U.S. Provisional Patent Application No. 62/875,915, filed Jul. 18, 2019; U.S. Provisional Patent Application No. 62/882,981, filed Aug. 5, 2019; and U.S. Provisional Patent Application No. 62/883,025, filed Aug. 5, 2019; each of which is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under contract AG020961 awarded by the National Institutes of Health. The Government has certain rights in the invention.

### BACKGROUND

In muscle wasting diseases a rapid loss of muscle mass and strength occurs due primarily to excessive protein degradation, which frequently is accompanied by diminished protein synthesis. Quality of life is reduced, and morbidity and mortality are increased due to this loss of muscle function. While much is known about how muscle atrophy arises, current therapeutic strategies to effectively prevent or slow atrophy are limited to exercise. A plausible strategy to increase muscle mass and strength is to alter protein balance, e.g. via modulation of the TGF-β family, or the insulin receptor signaling pathways.

Prostaglandin E2 (PGE2), also known as dinoprostone, has been employed in various clinical settings including to induce labor in women and to augment hematopoietic stem cell transplantation. PGE2 can be used as an anticoagulant and antithrombotic agent. PGE2's role as a lipid mediator that can resolve inflammation is also well known. Nonsteroidal anti-inflammatory drugs (NSAIDs), inhibitors of COX-1 and/or COX-2, suppress inflammation by inhibiting prostanoids, mainly via PGE2 biosynthesis. PGE2 is synthesized from arachidonic acid by a cyclooxygenase (COX) and prostaglandin E synthase enzymes. Levels of PGE2 are physiologically regulated by the PGE2 degrading enzyme, 15-hydroxyprostaglandin dehydrogenase (15-PGDH). 15-PGDH catalyzes the inactivating conversion of the PGE2 15-OH to a 15-keto group.

There remains a need in the art for effective treatments for preventing or reversing the loss of protein in aging and/or atrophied muscles, and the resulting loss of myofiber and/or myotube size and consequent loss of strength, endurance, or mass of atrophied muscle in a subject in need thereof. There also remains a need in the art for effective treatments for preventing or reversing loss of function in tissues, e.g., non-skeletal muscle tissues, in subjects with age-related diseases and disorders. The present disclosure satisfies these needs and provides other advantages as well.

### BRIEF SUMMARY

In one aspect, a method of enhancing a function of an aged skeletal muscle in a subject is provided, the method comprising: administering to the aged skeletal muscle a 15-PGDH inhibitor in an amount effective to inhibit 15-PGDH activity and/or reduce 15-PGDH levels in one or more senescent cells in the aged skeletal muscle, thereby enhancing a function of the aged skeletal muscle.

In another aspect, a method of increasing muscle mass, muscle strength, and/or muscle endurance of an aged skeletal muscle in a subject is provided, the method comprising: administering to the aged skeletal muscle a 15-PGDH inhibitor in an amount effective to inhibit 15-PGDH activity and/or reduce 15-PGDH levels in one or more senescent cells in the aged skeletal muscle, thereby increasing muscle mass, muscle strength, and/or muscle endurance of the aged skeletal muscle.

In another aspect, a method of increasing a level of PGE2 in an aged skeletal muscle in a subject is provided, the method comprising: administering to the aged skeletal muscle a 15-PGDH inhibitor in an amount effective to increase PGE2 levels in the aged skeletal muscle, thereby increasing a level of PGE2 in the aged skeletal muscle.

In any one of the preceding methods, the subject has one or more biomarkers of aging.

In yet another aspect, a method of rejuvenating an aged skeletal muscle in a subject having one or more biomarkers of aging is provided, the method comprising: administering to the subject having one or more biomarkers of aging a 15-PGDH inhibitor in an amount effective to inhibit 15-PGDH activity and/or reduce 15-PGDH levels in the subject, thereby rejuvenating the aged skeletal muscle.

In any one of the preceding methods, the one or more biomarkers of aging is selected from the group consisting of: an increase in 15-PGDH levels relative to a level present in young skeletal muscle, a decrease in PGE2 levels relative to a level present in young skeletal muscle, an increase in a PGE2 metabolite relative to a level present in young skeletal muscle, an increase or a greater accumulation of senescent cells relative to a level present in young skeletal muscle, an increase in expression of one or more atrogenes relative to a level present in young skeletal muscle, a decrease in mitochondria biogenesis and/or function relative to a level present in young skeletal muscle, and an increase in transforming growth factor pathway signaling relative to a level present in young skeletal muscle. In some cases, the one or more atrogenes is selected from the group consisting of: Atrogin1 (MAFbx1), MuSA (Fbxo30), and Trim63 (MuRFI). In some cases, the increase in transforming growth factor pathway signaling comprises an increase in expression of one or more gene selected from the group consisting of: Activin receptor, Myostatin, a SMAD protein, and a bone morphogenetic protein. In any one of the preceding methods, the aged skeletal muscle has an increased accumulation of senescent cells relative to young skeletal muscle. In some cases, the senescent cells express one or more senescent markers. In some cases, the senescent cells have an increased level of one or more senescent markers relative to non-senescent cells. In some cases, the one or more senescent markers is selected from the group consisting of: p15Ink4b, p16Ink4a, p19Arf, p21, Mmp13, Ill1, Il1b, and I16. In some cases, the senescent cells are macrophages. In any one of the preceding methods, the aged skeletal muscle is uninjured and/or has not undergone exercise and/or has not undergone regeneration. In any one of the preceding methods, the method further comprises administering a senolytic agent to the aged skeletal muscle. In some cases, the senolytic agent is selected from the group consisting of: a Bc12 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof. In any one of the preceding methods, the 15-PGDH inhibitor is selected from the group consisting of: a small molecule compound, a blocking antibody, a nanobody, and a peptide. In any one of the preceding methods, the 15-PGDH inhibitor is SW033291. In any one of the preceding methods, the 15-PGDH inhibitor is selected from the group consisting of: an antisense oligonucleotide, microRNA, siRNA, and shRNA. In any one of the preceding methods, the subject is a human. In any one of the preceding methods, the subject is at least 30 years of age. In any one of the preceding methods, the administering comprises systemic administration or local administration. In any one of the preceding methods, a level of PGE2 is increased in the aged skeletal muscle relative to a level of PGE2 present in the aged skeletal muscle prior to the administering of the 15-PGDH inhibitor. In any one of the preceding methods, a level of PGE2 is increased by at least 10% relative to a level of PGE2 present in the aged skeletal muscle prior to the administering of the 15-PGDH inhibitor. In any one of the preceding methods, a level of PGE2 is increased to a level that is substantially similar to a level present in young skeletal muscle. In any one of the preceding methods, a level of PGE2 is increased to a level that is within about 50% or less of a level present in young skeletal muscle. In any one of the preceding methods, the method results in an increase in myofiber and/or myotube cross-sectional area and/or diameter. In any one of the preceding methods, the method results in an increase in cross-sectional area and/or diameter of oxidative (type IIa) and/or glycolytic (type IIb) fibers. In any one of the preceding methods, the 15-PGDH inhibitor reduces or blocks 15-PGDH expression. In any one of the preceding methods, the 15-PGDH inhibitor reduces or blocks enzymatic activity of 15-PGDH. In any one of the preceding methods, the method results in an increase in muscle mass, an increase in muscle strength, an increase in muscle endurance, or any combination thereof of the aged skeletal muscle. In any one of the preceding methods, the method results in an increase in muscle mass, an increase in muscle strength, an increase in muscle endurance, or any combination thereof of the aged skeletal muscle relative to the aged skeletal muscle prior to the administering of the 15-PGDH inhibitor. In any one of the preceding methods, the method results in an increase in muscle mass, an increase in muscle strength, an increase in muscle endurance, or any combination thereof of the aged skeletal muscle to a level substantially similar to a level present in young skeletal muscle. In any one of the preceding methods, the method results in an increase in muscle mass, an increase in muscle strength, an increase in muscle endurance, or any combination thereof of the aged skeletal muscle to a level within about 50% or less of a level present in young skeletal muscle. In any one of the preceding methods, the method results in an enhanced function of the aged skeletal muscle. In any one of the preceding methods, the method results in an enhanced function of the aged skeletal muscle relative to the aged skeletal muscle prior to the administering of the 15-PGDH inhibitor. In any one of the preceding methods, the method results in an enhanced function of the aged skeletal muscle to a level substantially similar to a level present in young skeletal muscle. In any one of the preceding methods, the method results in an enhanced function of the aged skeletal muscle to a level within about 50% or less of a level present in young skeletal muscle. In any one of the preceding methods, the function is an increase in protein synthesis, an increase in cell proliferation, an increase in cell survival, a decrease in protein degradation, or any combination thereof. In any one of the preceding methods, the method results in decreased levels of a PGE2 metabolite in the aged skeletal muscle relative to the aged skeletal muscle prior to the administering of the 15-PGDH inhibitor, and/or to a level substantially similar to a level present in young skeletal muscle. In some cases, the PGE2 metabolite is selected from the group consisting of: 15-keto PGE2 and 13,14-dihydro-15-keto PGE2. In any one of the preceding methods, the subject has sarcopenia due to aging. In any one of the preceding methods, an expression level of one or more atrogenes is decreased relative to the aged skeletal muscle prior to the administering of the 15-PGDH inhibitor and/or to a level substantially similar to a level present in young skeletal muscle. In any one of the preceding methods, an expression level of one or more components of a mitochondria complex is increased relative to the aged skeletal muscle prior to the administering of the 15-PGDH inhibitor and/or to a level substantially similar to a level present in young skeletal muscle. In some cases, the one or more components of a mitochondria complex is selected from the group consisting of: Ndufa11, Ndufa12, Ndufa13, Ndufa2, Ndufa3, Ndufa4, Ndufa5, Ndufa10, Ndufb5, Ndufcl, Ndufs4, Ndufs8, Ndufv1, Ndufv2, Uqcrb, Uqcrcl, Uqcrh, Uqcrq, Ucqr10, Cox8b, Cox7a1, Cox7a2, Cox7b, Cox6c, Cox5a, Cox5b, Atp5fl, Atp5gl, Atp5h, Atp5j2, Atp5o, Atp5e, and Atp5k. In any one of the preceding methods, an expression level of peroxisome proliferator-activated receptor gamma coactivator 1-alpha (Pgc1α) is increased relative to the aged skeletal muscle prior to the administering of the 15-PGDH inhibitor and/or to a level substantially similar to a level present in young skeletal muscle. In any one of the preceding methods, an expression level of one or more genes selected from the group consisting of Tnfaipl, Klhdc8a, Fbxwll, Tnfaip3, Herc3, Herc2, Hdac4, Traf6, Ankibl, Mib1, Pja2, Ubr3, Thbsl, Smad3, Acvr2a, Rgmb, Tgfb2, and Mstn is decreased relative to the aged skeletal muscle prior to the administering of the 15-PGDH inhibitor and/or to a level substantially similar to a level present in young skeletal muscle. In any one of the preceding methods, the method is independent of an increase in proliferation of muscle stem cells (MuSCs) in the subject. In any one of the preceding methods, the administering comprises once a day, twice a day, once a week, or once a month administration.

In yet another aspect, a method of rejuvenating an aged non-skeletal muscle tissue in a subject is provided, the method comprising: administering to the subject an amount of a 15-PGDH inhibitor effective to inhibit 15-PGDH activity and/or reduce 15-PGDH levels in the subject, thereby rejuvenating the aged non-skeletal muscle tissue. In some cases, the administering increases a level of PGE2 in the aged non-skeletal muscle tissue of the subject. In some cases, a level of PGE2 in the aged non-skeletal muscle tissue is increased relative to the aged non-skeletal muscle tissue prior to the administering of the 15-PGDH inhibitor. In some cases, a level of PGE2 in the aged non-skeletal muscle tissue is increased by at least 10% relative to the aged non-skeletal muscle tissue prior to the administering of the 15-PGDH inhibitor. In some cases, a level of PGE2 in the aged non-skeletal muscle tissue is increased to a level substantially similar to a level present in young non-skeletal muscle tissue. In some cases, a level of PGE2 in the aged non-skeletal muscle tissue is increased to a level within about 50% or less of a level present in young non-skeletal muscle tissue. In some cases, the aged non-skeletal muscle tissue is selected from the group consisting of: epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof. In some cases, the subject has one or more biomarkers of aging. In some cases, the one or more biomarkers of aging is selected from the group consisting of: an increase in 15-PGDH levels relative to young non-skeletal muscle tissue, a decrease in PGE2 levels relative to young non-skeletal muscle tissue, an increase in a PGE2 metabolite relative to young non-skeletal muscle tissue, an increase or a greater accumulation of senescent cells relative to young non-skeletal muscle tissue, an increase in expression of one or more atrogenes relative to young non-skeletal muscle tissue, a decrease in mitochondria biogenesis and/or function relative to young non-skeletal muscle tissue, and an increase in transforming growth factor pathway signaling relative to young non-skeletal muscle tissue. In some cases, the aged non-skeletal muscle tissue has an increased accumulation of senescent cells relative to young non-skeletal muscle tissue. In some cases, the senescent cells express one or more senescent markers. In some cases, the senescent cells have an increased level of one or more senescent markers relative to non-senescent cells. In some cases, the one or more senescent markers is selected from the group consisting of: p15Ink4b, p16Ink4a, p19Arf, p21, Mmp13, I11a, I11b, and 116. In some cases, the senescent cells are macrophages. In some cases, the method further comprises administering a senolytic agent to the aged non-skeletal muscle tissue. In some cases, the senolytic agent is selected from the group consisting of: a Bc12 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof. In some cases, the 15-PGDH inhibitor is selected from the group consisting of: a small molecule compound, a blocking antibody, a nanobody, and a peptide. In some cases, the 15-PGDH inhibitor is SW033291. In some cases, the 15-PGDH inhibitor is selected from the group consisting of: an antisense oligonucleotide, microRNA, siRNA, and shRNA. In some cases, the subject is a human. In some cases, the subject is at least 30 years of age. In some cases, the 15-PGDH inhibitor reduces or blocks 15-PGDH expression. In some cases, the 15-PGDH inhibitor reduces or blocks enzymatic activity of 15-PGDH. In some cases, a function of the aged non-skeletal muscle is enhanced relative to a function of the aged non-skeletal muscle prior to the administering of the 15-PGDH inhibitor. In some cases, a function of the aged non-skeletal muscle tissue is enhanced by at least 10% relative to the function of the aged non-skeletal muscle prior to the administering of the 15-PGDH inhibitor. In some cases, a function of the aged non-skeletal muscle tissue is enhanced to a level that is substantially similar to a level present in young non-skeletal muscle tissue. In some cases, a function of the aged non-skeletal muscle tissue is enhanced to a level that is within about 50% or less of a level present in young non-skeletal muscle tissue. In some cases, the function comprises increased protein synthesis, increased cell proliferation, increased cell survival, decreased protein degradation, or any combination thereof. In some cases, the method results in decreased levels of a PGE2 metabolite in the aged non-skeletal muscle tissue relative to the aged non-skeletal muscle tissue prior to the administering of the 15-PGDH inhibitor and/or to a level that is substantially similar to a level present in young non-skeletal muscle. In some cases, the PGE2 metabolite is selected from the group consisting of: 15-keto PGE2 and 13,14-dihydro-15-keto PGE2.

In yet another aspect, a method of enhancing a function of a skeletal muscle in a subject is provided, the method comprising: administering to the subject a 15-PGDH inhibitor in an amount effective to inhibit 15-PGDH activity and/or reduce 15-PGDH levels in the skeletal muscle, thereby enhancing a function of the skeletal muscle in the subject, wherein the skeletal muscle is healthy, and wherein the method is independent of an increase in proliferation of muscle stem cells (MuSCs) in the subject. In some cases, the skeletal muscle is uninjured. In some cases, the skeletal muscle is not undergoing regeneration. In some cases, the skeletal muscle has not undergone significant or substantial exercise. In some cases, the function is enhanced relative to the skeletal muscle prior to the administering of the 15-PGDH inhibitor. In some cases, the function is an increase in protein synthesis, an increase in cell proliferation, an increase in cell survival, a decrease in protein degradation, or any combination thereof. In some cases, the method results in an increase in muscle mass, an increase in muscle strength, an increase in muscle endurance, or any combination thereof relative to the skeletal muscle prior to the administering of the 15-PGDH inhibitor. In some cases, the skeletal muscle is young skeletal muscle. In some cases, the subject is less than 30 years of age. In some cases, the skeletal muscle is aged skeletal muscle. In some cases, the subject is greater than 30 years of age.

In another aspect, the present disclosure provides a method for increasing the mass, strength, and/or endurance of aged and/or atrophied muscle in a subject, the method comprising administering to the subject a therapeutically effective amount of a 15-hydroxyprostaglandin dehydrogenase (15-PGDH) inhibitor, wherein the administration of the 15-PGDH inhibitor increases the myofiber and/or myotube size in the aged and/or atrophied muscle of the subject.

In some embodiments, the subject has a condition or disease associated with muscle atrophy selected from the group consisting of sarcopenia, diabetes, muscular dystrophy, sarcopenic obesity, neuropathy, cancer cachexia, HIV cachexia, muscle immobilization, muscle disuse, frailty, and combinations thereof. In some embodiments, the subject is a human. In some embodiments, the human is over 30 years of age (e.g., an adult with age-related sarcopenia). In some embodiments, the human is a child (e.g., a child with a muscular dystrophy such as Duchenne muscular dystrophy). In some embodiments, the method further comprises a step in which the human is selected for treatment with the 15-PGDH inhibitor based on his or her age.

In some embodiments, the method further comprises a step in which the human is selected for treatment with the 15-PGDH inhibitor based on a diagnosis of diabetes, frailty, muscular dystrophy, sarcopenic obesity, neuropathy, cancer cachexia, or HIV cachexia, or muscle atrophy resulting from immobilization or disuse. In some embodiments, the muscular dystrophy is selected from the group consisting of Duchenne muscular dystrophy, Becker muscular dystrophy, congenital muscular dystrophy, distal muscular dystrophy, Emery-Dreifuss muscular dystrophy, facioscapulohumeral muscular dystrophy, limb-girdle muscular dystrophy, myotonic muscular dystrophy, and oculopharyngeal muscular dystrophy. In some embodiments, the muscular dystrophy is Duchenne muscular dystrophy.

In some embodiments, the 15-PGDH inhibitor inactivates 15-PGDH or blocks 15-PGDH activity (e.g., enzymatic activity). In some embodiments, the 15-PGDH inhibitor reduces the stability of 15-PGDH. In some embodiments, the 15-PGDH inhibitor is a small molecule compound, blocking antibody, nanobody, or peptide. In some embodiments, the small molecule compound is SW033291. In some embodiments, the 15-PGDH inhibitor reduces or blocks 15-PGDH expression. In some embodiments, the 15-PGDH inhibitor is an antisense oligonucleotide, microRNA, siRNA, or shRNA. In some embodiments, the 15-PGDH inhibitor is a modified RNA, e.g., modified mRNA (mmRNA).

In some embodiments, the muscle is skeletal muscle. In some embodiments, the muscle is uninjured and/or has not undergone exercise and/or regeneration. In some embodiments, the inhibitor increases the myofiber and/or myotube size in the aged and/or atrophied muscle of the subject independent of muscle injury, exercise, or regeneration. In some embodiments, the therapeutically effective amount of the 15-PGDH inhibitor increases the muscle mass or the myofiber and/or myotube cross-sectional area or diameter in the aged and/or atrophied muscle of the subject. In some embodiments, the therapeutically effective amount of the 15-PGDH inhibitor increases muscle strength, muscle function, muscle mass, and/or muscle endurance independently of or without requiring an increase in proliferation of muscle stem cells (MuSCs) in the subject. In some embodiments, the therapeutically effective amount of the 15-PGDH inhibitor increases, elevates or restores prostaglandin E2 (PGE2) levels in the aged and/or atrophied muscle of the subject. In some embodiments, the therapeutically effective amount of the 15-PGDH inhibitor decreases PGE2 metabolite levels in the aged and/or atrophied muscle of the subject.

In some embodiments, the PGE2 metabolite is 15-keto-PGE2 or 13,14-dihydro-15-keto-PGE2 (PGEM). In some embodiments, administering the 15-PGDH inhibitor comprises systemic or local administration. In some embodiments, the aged and/or atrophied muscle has an increased accumulation of senescent cells (e.g., relative to young muscle).

In some embodiments, the method further comprises administering a senolytic agent to the subject. In some embodiments, the senolytic agent is selected from the group consisting of a Bc12 inhibitor (e.g., navitoclax (ABT-263), ABT-737), a pan-tyrosine kinase inhibitor (e.g., dasatinib), a flavonoid (e.g., quercetin), a peptide that interferes with the FOXO4-p53 interaction (e.g., FOXO4-DRI), a selective targeting system of senescent cells using galactooligosaccharide-coated nanoparticles, an HSP90 inhibitor (e.g., 17-DMAG), and combinations thereof.

In some embodiments, the administration of the 15-PGDH inhibitor results in a decrease in Atrogin1 levels or activity in the aged and/or atrophic muscle of the subject. In some embodiments, the administration of the 15-PGDH inhibitor results in an increase in EP4 activity in the aged and/or atrophic muscle of the subject. In some embodiments, the administration of the 15-PGDH inhibitor results in protection against muscle cell death, in particular of mature muscle cells.

The present disclosure provides compositions and methods for improving the health, function, and/or performance of non-skeletal muscle tissues in subjects with age-related conditions or diseases, in particular by inhibiting 15-PGDH in the subjects.

In one aspect, the present disclosure provides a method for increasing the function of a non-skeletal muscle tissue in a subject with an age-related disorder, the method comprising administering to the subject a therapeutically effective amount of a 15-hydroxyprostaglandin dehydrogenase (15-PGDH) inhibitor, wherein the administration of the 15-PGDH inhibitor increases or restores the level of PGE2 and/or PGD2 in the non-skeletal muscle tissue in the subject.

In some embodiments of the method, the age-related disorder is selected from the group consisting of cardiovascular disease, chronic respiratory disease, nutritional disease, kidney disease, gastrointestinal or digestive disease, neurological disorder, sensory disorder, hearing disorder, skin or subcutaneous disease, cerebrovascular disease, osteoporosis, osteoarthritis, premature aging disease, and combinations thereof. In some embodiments, the cardiovascular disease is atrial fibrillation, stroke, ischemic heart disease, cardiomyopathy, endocarditis, intracerebral hemorrhage, hypertension, or a combination thereof. In some embodiments, the chronic respiratory disease is chronic obstructive pulmonary disease, asbestosis, silicosis, or a combination thereof. In some embodiments, the nutritional disease is trachoma, diarrheal disease, encephalitis, or a combination thereof. In some embodiments, the kidney disease is a chronic kidney disease. In some embodiments, the gastrointestinal or digestive disease is NASH, pancreatitis, ulcer, intestinal obstruction, or a combination thereof. In some embodiments, the neurological disorder is Alzheimer's disease, dementia, Parkinson's disease, or a combination thereof. In some embodiments, the sensory disorder is hearing loss, vision loss, loss of sense of smell or sense of taste, macular degeneration, retinosa pigmentosa, glaucoma, or a combination thereof. In some embodiments, the skin or subcutaneous disease is cellulitis, ulcer, fungal skin disease, pyoderma, or a combination thereof. In some embodiments, the premature aging disease is Osteogenesis imperfecta, Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, Rothmund-Thomson syndrome, an immunodeficiency leading to a premature aging syndrome such as Ataxia telangiectasia, or an infectious disease leading to premature aging such as HIV.

In some embodiments of the method, the subject is a human. In some embodiments, the method further comprises a step in which the human is selected for treatment with the 15-PGDH inhibitor based on a diagnosis of the age-related disorder. In some embodiments, the non-skeletal muscle tissue is selected from the group consisting of epidermal, epithelial, vascular, cardiac muscle, brain, bone, cartilage, sensory organs, kidney, thyroid, lung, smooth muscle, brown fat, spleen, liver, heart, brain, small intestine, colon, skin, ovaries and other reproductive tissues, hair, dental tissues, cochlea, oligodendrocytes, and combinations thereof.

In some embodiments of the method, the 15-PGDH inhibitor inactivates 15-PGDH or blocks 15-PGDH activity. In some embodiments, the 15-PGDH inhibitor reduces or blocks the enzymatic activity of 15-PGDH. In some embodiments, the 15-PGDH inhibitor is a small molecule compound, blocking antibody, nanobody, or peptide. In some embodiments, the small molecule compound is SW033291. In some embodiments, the 15-PGDH inhibitor reduces or blocks 15-PGDH expression. In some embodiments, the 15-PGDH inhibitor is an antisense oligonucleotide, microRNA, siRNA, or shRNA.

In some embodiments of the method, the administration of the 15-PGDH inhibitor increases or restores the level of PGE2 in the non-skeletal muscle tissue in the subject. In some embodiments, the therapeutically effective amount of the 15-PGDH inhibitor decreases PGE2 and/or PGD2 metabolite levels in the non-skeletal muscle tissue of the subject. In some embodiments, the PGE2 metabolite is 15-keto-PGE2 or 13,14-dihydro-15-keto-PGE2 (PGEM). In some embodiments, the PGD2 metabolite is 15-keto-PGD2 or 13,14-dihydro-15-keto-PGD2. In some embodiments, the therapeutically effective amount of the 15-PGDH inhibitor increases protein synthesis, increases cell proliferation, increases cell survival, lengthens telomeres, and/or decreases protein degradation, in the non-skeletal muscle tissue of the subject. In some embodiments, administering the 15-PGDH inhibitor comprises systemic administration. In some embodiments, administering the 15-PGDH inhibitor comprises local administration. In some embodiments, the non-skeletal muscle tissue has an increased accumulation of senescent cells (e.g., relative to young non-skeletal muscle tissue). In some embodiments, the method further comprises administering a senolytic agent to the subject. In some embodiments, the senolytic agent is selected from the group consisting of a Bc12 inhibitor, a pan-tyrosine kinase inhibitor, a flavonoid, a peptide that interferes with the FOXO4-p53 interaction, a selective targeting system of senescent cells using galactooligosaccharide-coated nanoparticles, an HSP90 inhibitor, and combinations thereof.

Other objects, features, and advantages of the present disclosure will be apparent to one of skill in the art from the following detailed description and figures.

### BRIEF DESCRIPTION OF THE DRAWINGS

FIGS. 1A-ID. Decline in strength and PGE2 levels in aged muscles. (FIG. 1A) Plantar flexion muscle tetanic torque in young (2 months, n=9), mid (18 months, n=5), and aged (25 months, n=5) male mice. (FIG. 1B) PGE2 catabolism scheme. 13,14-dihydro-15-keto PGE2 (PGEM). (FIG. 1C) 15-PGDH specific enzymatic activity assayed in muscle tissues of young (2 months) and aged (25 months) mice (n=4 mice per age group). (FIG. 1D) PGE2 and PGEM levels in muscle tissue lysates quantified by mass spectrometry (n=14 mice for young, and n=8 for aged). *P<0.05, **P<0.001, ****P<0.0001. ANOVA test with Bonferroni correction for multiple comparisons (FIGS. 1A and 1D); Mann-Whitney test (FIG. 1C). Means±s.e.m.

FIG. 2. 15-PGDH, a component of senescent cells in aged tissues. Expression of 15-PGDH (Hpgd) in muscle tissues of 20-month C57B1/6 wild type mice that were treated with vehicle (veh) or ABT-263 (ABT) over a 4-week alternating regimen and analyzed 2 months later (n=3 per condition in 2 month old mice and n=4 per condition in 23 month old mice). *P<0.05. ANOVA test with Bonferroni correction for multiple comparisons; Means+s.e.m. Abbreviation: mo, months.

FIGS. 3A-3E. 15-PGDH inhibition leads to improved muscle function in aged mice by increasing endogenous PGE2 levels. (FIG. 3A) Aged mice were treated daily with 15-PGDH inhibitor, SW033291 (SW), or vehicle and muscle function was measured at 1 month. Experimental Scheme (Top). Left to right: Mass assessed as weight of dissected gastroenemius (GA) and tibialis anterior (TA) muscles. Strength assessed as plantar flexion tetanic force (absolute values). Plantar flexion tetanic force (values normalized to baseline). Endurance assessed as time and distance to exhaustion. (FIG. 3B) Representative TA cross-section of 1 month vehicle treated or SW treated aged muscles. DAPI, blue; LAMININ, green. Bar=50 μm. (FIG. 3C) Myofiber cross-sectional areas (CSA) in vehicle- and SW-treated aged GAs (n=4 per group). (FIG. 3D) Mean CSA (n=4 per group). (FIG. 3E) PGE2 and PGEM levels in muscle tissue lysates quantified by mass spectrometry (n=3 per group).*P<0.05, **P<0.001, ****P<0.0001. Mann-Whitney test (FIGS. 3A and 3D). ANOVA test with Bonferroni correction for multiple comparisons (FIGS. 3C and 3E); Means±s.e.m. Abbreviations: mo, months; i.p. intraperitoneal.

FIGS. 4A-4D. 15-PGDH knockdown by AAV9-delivered shRNA leads to improved muscle function in aged mice. Intramuscular (i.m.) injection of AAV9 carrying a construct of an shRNA against 15-PGDH (sh15PGDH) or scramble (scr) control into the GA. (FIG. 4A) Experimental scheme. (FIG. 4B) Expression levels of 15-PGDH in scr and sh15PGDH infected muscles and young control (n=5 per group). (FIG. 4C) Weight of dissected gastrocnemius (GA). (FIG. 4D) Plantar flexion tetanic force (absolute values).*P<0.05. ANOVA test with Bonferroni correction for multiple comparisons (FIG. 4B); Mann-Whitney test (FIGS. 4C and 4D). Means±s.e.m. Abbreviations: mo, months; i.m. intramuscular.

FIGS. 5A and 5B. 15-PGDH inhibition leads to improved muscle function in a Duchenne Muscular Dystrophy mouse model. (FIG. 5A) Expression of senescence markers and 15-PGDH (Hpgd) in the GA muscles of Duchenne Muscular Dystrophy (DMD) mice (mdx4cv/mTRKO(G2)) and controls (mTRKO(G2))(n=4 per genotype). (FIG. 5B) DMD mice and control mice were treated daily with 15-PGDH inhibitor, SW033291 (SW), or vehicle and muscle function was measured at 1 month. Experimental scheme (top). Plantar flexion tetanic force (values normalized to vehicle-treated for each genotype, bottom).*P<0.05, ****P<0.0001. Mann-Whitney test (FIGS. 5A and 5B). Means±s.e.m. Abbreviations: mo, months; i.p. intraperitoneal.

FIGS. 6A-6F. PGE2 treatment of cultured myotubes leads to inhibition of the muscle atrophy pathway. (FIG. 6A) Expression levels of Atrogin1 in: (left) vehicle and SW treated aged muscles (n=3 per condition); (right) shscr and sh15PGDH treated aged muscles (n=5 per condition). (FIG. 6B) Expression levels of PGE2 receptors, EP1-4 (Ptger1-4) during a timecourse of differentiation. (FIG. 6C) Expression levels of Pax7 and Myh during a timecourse of differentiation. (FIG. 6D) Expression levels of atrophy marker, Atrogin1 (left), and myotube diameter (middle) in differentiated myotubes starved for 24 hr and concomitantly treated with vehicle, PGE2 or SW in the presence of the EP4 antagonist, ONO-AE3-208. Representative images of myotubes exposed to PGE2 or vehicle post-differentiation (right). Bar=50 μm. (FIG. 6E) Diameter and MYH stained positive area of EP4fl/fl or EP4A/A myotubes. (FIG. 6F) Graphic description of 15-PGDH regulation in aged and dystrophic mice. Rescue of muscle mass and strength loss in aged or DMD muscles can be achieved by use of a 15-PGDH inhibitor or senolytics to restore levels of PGE2, resulting in decreased levels of downstream atrophy mediator Atrogin1, muscle hypertrophy and increased strength in treated DMD or aged mice. *P<0.05, **P<0.001, ***P<0.0005 ****P<0.0001. Mann-Whitney test (FIG. 6A, 6D-left, and 6E); ANOVA test with Bonferroni correction for multiple comparisons (FIG. 6D-right); Means±s.e.m.

FIGS. 7A-7C. Mass spectrometry analysis of young and aged muscle to detect prostaglandins and PGE2 metabolites. (FIG. 7A) Chemical structures, chemical formula, exact mass and molecular weight of analyzed prostaglandins (PGE2, PGF2a and PGD2) and PGE2 metabolites (15-keto PGE2 and 13,14-dihydro-15-keto PGE2). The internal standards PGF2α-D9 and PGE2-D9 were added to all composite standards. (FIG. 7B) Calibration lines for liquid chromatography-electrospray ionization-tandem mass spectrometry (LC-ESI-MS/MS) analysis were prepared by diluting stock solutions to final concentrations of 0.1 ng/ml to 500 ng/ml. Standard curve equations and correlation coefficients are shown for each standard. (FIG. 7C) Representative chromatogram. The separate peaks show excellent chromatographic resolution of the analyzed prostaglandins and their metabolites. cps: counts per second.

FIGS. 8A-8P. Analysis of eicosanoid levels during aging uncovered an increase in PGE2 degrading enzyme 15-PGDH. (FIG. 8A) PGE2 and PGD2 catabolism scheme. (FIG. 8B) PGE2, PGD2, PGF2a and 13,14-dihydro-15-keto PGE2 (PGEM) levels in muscle tissue lysates quantified by mass spectrometry (n=12 mice for young, and n=8 for aged) (FIG. 8C) Representative chromatogram of the PGE2, PGD2 levels analyzed by mass spectrometry from young (2 months, left) and aged (25 months, right) muscle tissues. (FIG. 8D) 15-PGDH specific enzymatic activity assayed in tissues of young (2 months) and aged (25 months) mice. Activity is expressed as percent change relative to young. (FIG. 8E) 15-PGDH (Hpgd) RNAseq expression data from young (3 mo.) and aged mice (>24mo.) (n=4, 6 respectively). TPM, transcripts per million. (FIG. 8F) 15-PGDH immunoblots from muscle lysates of young (3 month) and aged (25 month) (n=4 each). (FIGS. 8G-8P) Intramuscular (i.m.) injection of AAV9 carrying a construct of an shRNA against 15-PGDH (sh15PGDH) or scramble (scr) control into the Gastocnemius (GA) of young (3 month) and aged (24 month) old C57BL/6. (FIG. 8G) Experimental scheme. (FIG. 8H) Expression levels of 15-PGDH in scr and sh15PGDH infected muscles and young control (n=5 per group). (FIG. 8I) 15-PGDH specific enzymatic activity assayed in muscle tissues of scr and sh15PGDH infected aged muscles normalized to scr treated (n=5 mice per age group). (FIG. 8J) PGE2, PGD2, PGF2a levels in muscle tissue lysates quantified by mass spectrometry (n=4 per group). (FIG. 8K) Representative TA cross-section of scr and sh15PGDH infected aged muscles. DAPI, blue; LAMININ, green. Bar=50 μm. (FIG. 8L) Myofiber cross-sectional areas (CSA) in scr and sh15PGDH infected aged GAs (n=7 per group). (FIG. 8M) Mean CSA. (FIG. 8N) Weight of dissected TA (FIG. 8O) Weight of dissected GA. (FIG. 8P) Plantar flexion tetanic force (absolute values). *P<0.05, **P<0.01, ****P<0.0001. ANOVA test with Bonferroni correction for multiple comparisons (FIGS. 8H and 8L-8P); Multiple t-tests (FIGS. 8B, 8D, and 8J), Mann-Whitney test (FIGS. 8E, 8F, and 8I). Means±s.e.m. Abbreviations: Spl. Spleen; Mus. Muscle; mo months; i.m. intramuscular.

FIGS. 9A-9C. Mass spectrometry analysis of young and aged muscle detects prostaglandins and PGE2 metabolites. (FIG. 9A) Chemical structures, chemical formula, exact mass and molecular weight of analyzed prostaglandins (PGE2, PGF2α and PGD2), PGE2 metabolites (15-keto PGE2 and 13,14-dihydro-15-keto PGE2), PGA2 and its metabolite, 13,14-dihydro-15-keto PGA2, and internal standards PGF2α-D9 and PGE2-D4 and PGD2-D4. (FIG. 9B) PGE2 calibration curve was linear in the range 0.05-500 ng/mL. Standard curve equation and correlation coefficient are shown. (FIG. 9C) Representative chromatogram of a standard mix showing chromatographic separation of the analyzed prostaglandins and their metabolites. Analyte peak intensities are expressed as cps, counts per second.

FIG. 10. Mass spectrometry analysis of young and aged muscle. Representative chromatogram indicates transition states of the metabolite PGE2 levels analyzed by mass spectrometry from young (2 months, left) and aged (25 months, right) muscle tissues.

FIG. 11. 15-PGDH specific activity assay in young and aged tissues. Kinetic measurement of 15-PGDH specific activity in lysates prepared from young (grey) and aged (black) tissues.

FIGS. 12A-12D. Transcriptomic analysis of quadriceps from young vs aged C57BL/6. (FIGS. 12A-12D) RNA Sequencing was performed on young (3 mo.) and aged mice (>24mo.) (n=4, 6 respectively). (FIG. 12A) Heatmap of Euclidean sample distances of young and aged samples after rlog transformation (FIG. 12B) Volcano plot of differentially expressed genes of young vs aged samples. (FIG. 12C) Box and whiskers plot of TPM values of Prostaglandin E2 receptors (Ptger 1-4). (FIG. 12D) GO term and KEGG analysis of differentially up- and down-regulated gene from (FIG. 12B). Abbreviation: mo., months; n.s., non significant; TPM Transcripts Per Million.

FIG. 13. 15-PGDH levels are elevated in aged muscles. 15-PGDH (Hpgd) microarray expression data from aged human (78±6 yrs) biopsies from the vastus lateralis muscle compared to young (25±3 yrs) (n=15, 21 respectively) analyzed from publicly available data GSE25941 (Raue et al. 2012). *P<0.0001. Mann-Whitney test.

FIGS. 14A-14C. AAV9 mediated knockdown of 15-PGDH. (FIG. 14A) Mass spectrometry quantification of PGE2, PGD2, PGF2a levels in muscle tissue of young sh15PGDH relative to shscr (n=4 per group). (FIG. 14B) Representative images of TA cross-section of scr and sh15PGDH infected aged muscles DAPI, blue; GFP, green; LAMININ, white (FIG. 14C) Plantar flexion tetanic force (relative to baseline). *P<0.05. Multiple t-test (FIG. 14A), ANOVA test with Bonferroni correction for multiple comparisons (FIG. 14C). Means+s.e.m. Abbreviation: TA: Tibialis anterior; scr: scrambled, n.s., non significant.

FIGS. 15A-15M. 15-PGDH inhibition by a small molecule leads to improved muscle function of aged mice by increasing endogenous PGE2 levels. (FIG. 15A) Experimental scheme. Young (3 month) and aged (>24 month) mice were treated daily with 15-PGDH inhibitor, SW033291 (SW) or vehicle and muscle function was measured at 1 month. (FIG. 15B) 15-PGDH specific enzymatic activity assayed in muscle tissues of vehicle and SW treated aged muscles normalized to vehicle treated (n=4 mice per age group). (FIG. 15C) Eicosanoid levels in muscle tissue lysates quantified by mass spectrometry (n=10 for young, n=5 for aged veh and n=7 for aged SW). (FIG. 15D) Representative TA cross-section of 1 month treated vehicle or SW treated aged muscles. DAPI, blue; LAMININ, green. Bar=50 μm. (FIG. 15E) Myofiber cross-sectional areas (CSA) in vehicle and SW treated aged GAs (n=4 per group). (FIG. 15F) Mean CSA. (FIG. 15G) Representative TA cross-section of 1 month treated vehicle or SW treated aged muscles stained for oxidative (MHC2a) and glycolytic fibers (MHC2b) LAMININ, Blue; MHC2a, green and MHC2b, Red Bar=50 μm (FIG. 15H) Mean CSA. (FIG. 15I) Cross-sectional area of MHC2a. n=4 per group (FIG. 15J) Cross-sectional area of MHC2b. n=4 per group (FIG. 15K) Weight of dissected Gastrocnemius (GA), Tibialis anterior (TA) and Soleus muscles. (FIG. 15L) Plantar flexion tetanic force (absolute values). (FIG. 15M) Time to exhaustion. *P<0.05, **P<0.01,****P<0.0001. Mann-Whitney test (FIGS. 15B and 15H); ANOVA test with Bonferroni correction for multiple comparisons (FIGS. 15C, 15E, 15F, and 15J-15M). Means±s.e.m. Abbreviation: mo, months; i.p. intraperitoneal.

FIGS. 16A-16C. Analysis of aged vehicle and SW treated muscle. (FIG. 16A) Representative chromatogram indicates transition states of the metabolite PGE2 levels analyzed by mass spectrometry from aged vehicle treated (left) and aged SW treated (right) muscle tissues. (FIG. 16B) Mass spectrometry quantification of PGE2, PGD2, PGF2a levels in muscle tissue of SW treated relative to Vehicle treated (n=4 per group). (FIG. 16C) Plantar flexion tetanic force (relative to baseline). **P<0.01. Multiple t-test (FIG. 16B), ANOVA test with Bonferroni correction for multiple comparisons (FIG. 16C). Abbreviation: n.s., non significant.

FIGS. 17A-17G. 15-PGDH is expressed by cells in the aged muscle microenvironment. (FIG. 17A) Expression of 15-PGDH (Hpgd) in sorted macrophages (Cd11b+/Cd11c-/F4/80+/Cd31-), endothelial (Cd31+/Cd11b-/Cd11c-/F4/80-) and myogenic and stem cells (α7+/Cd11b-/Cd45-/Cd31-/Scal-) from young (2 months) and aged (25 months) from the hindlimb muscles. (FIG. 17B) Expression of p16Ink4a and p21 in FACS isolated young (2 month) and aged macrophages (25 month) (n=3 and 5 respectively). (FIGS. 17C-17G) INK-ATTAC 12-month-old mice were treated with vehicle or AP20187 (AP) twice a week for 16 months to eliminate senescent cells and skeletal muscle tissues were analyzed at 28 months. (FIG. 17C) Experimental Scheme. (FIG. 17D) Expression of 15-PGDH enzyme (Hpgd) in the quadriceps muscle of young (2 months), and aged (28 months) INK-ATTAC mice treated with vehicle (veh) or AP. n=5 for 2 mo, n=6 for 28 months treated with veh or AP. (FIG. 17E) Eicosanoid levels in muscle tissue lysates quantified by mass spectrometry (n=10 for young, n=3 mice for vehicle-treated and n=3 for AP-treated). (FIG. 17F) Expression of 15-PGDH (Hpgd) in sorted macrophages and endothelial cells from adult (12 months) and aged INK-ATTAC treated with vehicle (veh) or AP (28 months) from the hindlimb muscles. (FIG. 17G) Weight of dissected Gastroenemius (GA), Tibialis anterior (TA) muscles (left), grip strength and treadmill endurance (right) of adult (12 months) and aged INK-ATTAC treated with vehicle (veh) or AP (28 months). n=6, 7 and 15 respectively.*P<0.05, ***P<0.001, ****P<0.0001. Multiple t-tests (A), ANOVA test with Bonferroni correction for multiple comparisons (FIGS. 17E-17G). Means±s.e.m.

FIGS. 18A and 18B. Expression of aging markers of sorted cells from young and aged mice. (FIG. 18A) Sorting of macrophages (Cd11b+/Cd11c-/F4/80+/Cd31-) from young (3mo.) and aged mice (24 mo.). (FIG. 18B) Expression of p16 and p21 in young (2 mo.) and aged (24 mo.) in sorted endothelial (Cd31+/Cd1 1b-/Cd11c-/F4/80-). (n=5 mice per condition). *P<0.05, ****P<0.0001. Mann-Whitney test (FIG. 18B). Means+s.e.m. Abbreviation: mo., months.

FIGS. 19A-19G. INK-ATTAC and senolytic treated aged mice characterization. (FIG. 19A) Expression of indicated senescence markers in the quadriceps muscle of young (2 months), and aged (28 months) INK-ATTAC mice treated with vehicle (veh) or AP. n=5 for young, n=6 for aged treated with veh or AP. (FIG. 19B) Representative chromatograms indicates transition states of the metabolite PGE2 levels analyzed by mass spectrometry from aged INK-ATTAC vehicle treated (left) and aged INK-ATTAC AP treated (right) muscle tissues. (FIG. 19C) Expression of p21 in sorted macrophages and endothelial cells of adult (12 mo.) INK-ATTAC, aged (28 mo.) INK-ATTAC mice treated with vehicle (veh) or AP. (n=4 per condition). (FIGS. 19D-19G) 20-month C57B1/6 wild type mice were treated with vehicle (veh) or ABT-263 (ABT) over a 4-week alternating regimen and analyzed 2 months later. (FIG. 19D) Scheme (top). Expression of senescent markers of young or aged C57B1/6 wild type (wt) mice treated with vehicle or ABT263 (ABT) and during a 4-week alternated regime (n=3 per condition in young mice and n=4 per condition in aged mice) (bottom). (FIG. 19E) Representative TA cross-section of young (2 months), aged ABT-treated and aged vehicle-treated muscles (23 months). DAPI, blue; 15-PGDH, green; WGA, red. (Bar=20 μm). (FIG. 19F) Quantification of 15-PGDH+immunostained cells in muscle tissues sections. (Muscle cross-sections (˜5,000-8,000 DAPI positive cells per section) for n=4 aged mice treated with ABT and n=4 mice treated with vehicle control) (FIG. 19G) Expression of 15-PGDH (Hpgd) (n=3 in young 2 month old mice and n=4 per condition in aged 23 month old mice) *P<0.05,**P<0.01, ***P<0.001. ANOVA test with Bonferroni correction for multiple comparisons (FIGS. 19A, 19C, 19D-left, and 19G). Mann-Whitney test (FIGS. 19F, and 19D-right). Means+s.e.m. Abbreviation: mo., months.

FIGS. 20A-20K. Overexpression of 15-PGDH induces muscle atrophy, rescued by treatment with SW033291. (FIGS. 20A-20H) Intramuscular (i.m.) injection of AAV9 carrying a construct of CMV driving 15-PGDH expression or control into the Tibialis anterior (TA) of young C57BL/6 (4 months) mice. (FIG. 20A) Experimental scheme. (FIG. 20B) Expression of 15-PGDH (Hpgd) in scr and 15-PGDH O.E. infected young muscles (n=5 per group). (FIG. 20C) PGE2, PGD2, PGF2a and PGEM levels in muscle tissue lysates quantified by mass spectrometry (n=4 per group). (FIG. 20D) Representative TA cross-section 1 month post i.m injection. DAPI, blue; LAMININ, green. Bar=50 μm (FIG. 20E) Myofiber cross sectional area of muscle injected with 15-PGDH overexpression vector and control (n=3 per group). (FIG. 20F) Weight of dissected Tibialis anterior (TA) muscles. (FIG. 20G) Plantar flexion tetanic force (absolute values). (FIG. 20H) Expression level of MuRFI (Trim63), Atrogin-1 (Fbxo32), p62, Lc3b, Atg4 and Atg6 measured by qPCR (n=3). (FIGS. 20I-20K) Intramuscular (i.m.) injection of AAV9 carrying a construct of CMV driving 15-PGDH expression or control into the Tibialis anterior (TA) of young C57BL/6 (3 months) mice together with daily intraperitoneal (i.p.) treatment with 15-PGDH inhibitor, SW033291 (SW) or vehicle (n=4 mice per group). (FIG. 20I) Experimental scheme. (FIG. 20J) Weight of dissected TA muscles. (FIG. 20K) Plantar flexion tetanic force (absolute values). *P<0.05, **P<0.01, ***P<0.001 ****P<0.0001. ANOVA test with Bonferroni correction for multiple comparisons (FIGS. 20J and 20K); Multiple t-tests (FIG. 20C), Mann-Whitney test (FIGS. 20B and 20E-20H). Means±s.e.m.

FIGS. 21A-21K. PGE2 mediates beneficial effects of 15-PGDH inhibition. (FIGS. 21A-21G) Intramuscular (i.m.) injection of AAV9 carrying a construct of an shRNA against Prostaglandin D2 Synthase, PTGDS (shPTGDS) or scramble (scr) control into the Gastrocnemius (GA) of aged (>24 month) old C57BL/6 mice. (FIG. 21A) Experimental scheme. (FIG. 21B) Expression of Ptgds measured by qPCR (n=4 per group). (FIG. 21C) PGD2 level in muscle tissue lysates quantified by mass spectrometry (n=4 per group). (FIG. 21D) Weight of dissected GA. (FIG. 21E) Plantar flexion tetanic force (values normalized to baseline). (FIG. 21F) Plantar flexion tetanic force (absolute values). (FIG. 21G) Distance to exhaustion on treadmill. (FIGS. 21H-21K) Intramuscular (i.m.) injection of AAV9 carrying a construct of MCK promoter driving Cre expression into the GA of EP4f/f mice or littermate controls (EP4+/+). Mice were then treated daily with 15-PGDH inhibitor, SW033291 (SW) or vehicle and muscle function was measured at 1 month. (FIG. 21H) Experimental scheme. (FIG. 21I) Weight of dissected GA (FIG. 21J) Plantar flexion tetanic force (values normalized to baseline). (FIG. 21K) Plantar flexion tetanic force (absolute values). *P<0.05,**P<0.01, ***P<0.001 ****P<0.0001. ANOVA test with Bonferroni correction for multiple comparisons (FIGS. 21B, 21D-21G, and 21I-21K); Mann-Whitney test (FIG. 21C). Means±s.e.m. Abbreviation: mo. months; i.p. intraperitoneal; i.m. intramuscular.

FIG. 22. Expression of prostaglandin receptors in myotubes. Expression levels of PGE2 receptors, EP1-4 (Ptger1-4), PGD2 receptors (Ptgdr1-2) and PGF2a receptor (Ptgfr) of myotubes (day 4 differentiated myotubes).

FIGS. 23A and 23B. PGE2 treatment leads to activation of CREB in muscles. (A) Immunoblots of muscle lysates from young (3 mo.) C57BL/6 mice injected with PGE2 i.m. after 0, 30 or 60 minutes. (B) Quantification of immunoblot in (A). **P<0.01. ANOVA test with Bonferroni correction for multiple comparisons (B). Means+s.e.m.

FIGS. 24A-24I. 15-PGDH inhibition impinges on multiple pathways to improve muscle function. (FIGS. 24A-24C) RNA sequencing analysis of aged muscle mice were treated daily with 15-PGDH inhibitor, SW033291 (SW) or vehicle and muscle function was measured at 1 month (n=3 each). (FIG. 24A) KEGG and GO Term analysis of upregulated (left) and downregulated (right) genes. (FIG. 24B) Heatmap of mitochondrial genes identified in (FIG. 24A). (FIG. 24C) Expression level of Pgc1a by qPCR (n=4 per group). (FIG. 24D) Relative quantification of mitochondrial DNA to nuclear DNA (n=4 per group). (FIG. 24E) Heatmap of protein ubiquitin related genes (top) and TGF-beta signaling pathway (bottom) identified in (FIG. 24A). (FIG. 24F) Immunoblots of myotubes (MT) differentiated from myogenic precursors derived from human muscle biopsies treated for 0, 15 or 30 min of PGE2 (10 ng/ml). (FIG. 24G) Immunoblots of muscle lysates from aged vehicle and SW treated mice (top) and quantification (bottom) (n=4 each). (FIG. 24H) Expression level of MuRF1 (Trim63), Atrogin-1 (Fbxo32) and Myostatin (Mstn) in Vehicle and SW treatment measured by qPCR (n=12 for aged veh and n=8 for aged SW). (FIG. 24I) Expression level of MuRF1 (Trim63), Atrogin-1 (Fbxo32) and Myostatin (Mstn) in scr and sh15PGDH treatment measured by qPCR (n=5 for aged shscr and n=4 for aged sh15PGDH).*P<0.05, **P<0.01,***P<0.001 ****P<0.0001. ANOVA test with Bonferroni correction for multiple comparisons (FIG. 24C); Mann-Whitney test (FIGS. 24D and 24G-I). Means±s.e.m. Abbreviation: KEGG: Kyoto Encyclopedia of Genes and Genomes; GO: Gene Ontology; BP: Biological Process; MF: Molecular Function; CC: Cellular Component.

FIGS. 25A-25D. PGE2 treatment leads to increased protein synthesis in myotubes. (FIG. 25A) Diameter of postdifferentiation myotubes starved for 24 hr and concomitantly treated with vehicle, PGE2 (10 ng/ml) or SW (1 μM) in the presence of the EP4 antagonist, ONO-AE3-208 (1 μM). (n=4 per condition) (FIG. 25B) Representative images of starved myotubes treated as in (FIG. 25A) DAPI, blue; MYH, red. Bar=50 μm. (FIG. 25C) Left: Diameter of postdifferentiation myotubes treated with vehicle or PGE2 for 4 days. Right: Representative image of postdifferentiation myotubes treated with vehicle or PGE2 for 4 days. DAPI, blue; Myosin Heavy Chain (MYH), red. Bar=50 μm. DM, differentiation medium. (FIG. 25D) Left: Immunoblot of puromycin incorporation into differentiated murine myotubes treated daily (4 d) with PGE2 (10 ng/ml) or vehicle. Cycloheximide was added as a control during puromycin addition. Right: The loading control is presented as the Ponceau S staining. ANOVA test with Bonferroni correction for multiple comparisons (FIG. 25A), Mann-Whitney test (FIG. 25B). ***P<0.001, ****P<0.0001. Means+s.e.m.

FIGS. 26A-26D. Characterization of 15-PGDH inhibition or knockdown in aged muscles. (FIG. 26A) Expression levels of atrophy markers in vehicle and SW treated aged muscles (n=8 and 5, respectively). (FIG. 26B) Expression levels of autophagy markers in young (3mo.) vehicle and SW treated aged muscles (n=4 for young, n=12 for aged veh and n=8 for aged SW). (FIG. 26C) Expression levels of inflammatory and senescent markers in vehicle and SW treated aged muscles (n=3 per condition). (FIG. 26D) Expression levels of inflammatory and senescent markers in shscr and sh15PGDH AAV9 treated aged muscles, (n=5 per condition). Mann-Whitney test (FIGS. 26A, 26C, and 26D), ANOVA test with Bonferroni correction for multiple comparisons (FIG. 26B), *P<0.05, **P<0.01. Means+s.e.m. Abbreviation: n.s., non significant.

FIGS. 27A and 27B. PGE2 degrading enzyme 15-PGDH is increased in aged tissues. (FIG. 27A) PGE2 and PGD2 catabolism scheme. (FIG. 27B) 15-PGDH specific enzymatic activity assayed in tissues of young (2 months) and aged (25 months) mice. Activity is expressed as percent change relative to young. *P<0.05, **P<0.001,***P<0.0005. Multiple t-tests (FIG. 27B). Means±s.e.m. Abbreviations: Spl. Spleen; Mus. Muscle.

FIG. 28. 15-PGDH specific activity assay of young and aged tissues. Kinetic measurement of 15-PGDH specific activity in lysates prepared from young (gray) and aged (black) tissues.

### DETAILED DESCRIPTION

### 1. Introduction

The present disclosure is based, in part, on the discovery that a loss of PGE2 signaling contributes to wasting of skeletal muscles during aging and muscular dystrophy and in association with muscle atrophy, and that PGE2 catabolism is dysregulated, leading to detrimental effects on aged, dystrophic, or atrophic muscle tissues. In aged muscle tissues, PGE2 is detected at lower levels, a phenomenon not previously associated with aging. Further, elevated PGE2 degrading enzyme, 15-PGDH, levels in aged or dystrophic muscles, due in part to an accumulation of senescent cells, lead to a reduction in muscle tissue PGE2 levels. The present disclosure therefore provides compositions and methods based on the use of 15-PGDH activity as a therapeutic target in aged and/or dystrophic muscle to improve, e.g., muscle atrophy, increasing muscle mass, function, and strength. In particular, reduction or inhibition of 15-PGDH (e.g., activity or levels, e.g., mRNA and/or protein) may lead to an improvement of skeletal muscle function in aging and muscular dystrophy. In one embodiment, the methods provided herein involve administering an inhibitor of 15-PGDH to treat aged and/or dystrophic muscles. In some cases, the methods involve increasing the levels of PGE2 (e.g., by inhibiting the PGE2 degrading enzyme, 15-PGDH) in aged, atrophic, or dystrophic muscles.

The elevation, increase, or restoration of PGE2 levels in aged, atrophic, or dystrophic muscles, e.g., in the absence of injury, exercise, or regeneration, may ameliorate muscle wasting, revealing a previously unrecognized role for the PGE2 degrading enzyme, 15-PGDH, in muscle wasting diseases such as muscular dystrophy, and in aging. In particular, PGE2 may act on mature myofibers in homeostasis in the absence of injury. Accordingly, 15-PGDH inhibitors (e.g., SW033291) may restore levels of PGE2 in aged, atrophic, and/or dystrophic skeletal muscles, together with decreased levels of the inactive PGE2 metabolites, e.g., PGEM. In some cases, the use of 15-PGDH inhibitors described herein may augment or enhance muscle mass, strength, exercise performance, and/or function. The pathway of PGE2 signaling may occur through the EP4 receptor in differentiated muscle cells and myofibers, and may directly regulate muscle mass through inhibition of Atrogin1 expression, a crucial mediator of muscle atrophy. 15-PGDH inhibition can be achieved by local or systemic strategies, surmounting the deleterious effects of the aged, atrophic, and dystrophic muscle microenvironment and leading to a robust increase in muscle mass, strength, and endurance in aged and dystrophic muscles.

The present disclosure is further based, in part, on the discovery that the PGE2 degrading enzyme, 15-PGDH, or its transcript, is elevated in a range of aging tissues, in particular, non-skeletal muscle tissues. As such, 15-PGDH proteins or transcripts can be used as a biomarker for aging in non-skeletal muscle tissues, e.g., in subjects with an age-related disorder or disease. In addition, 15-PGDH can be inhibited in order to reverse or slow aging and aging-related processes in non-skeletal muscle tissues, thereby ameliorating their function. Without being bound by the following theory, it is believed that elevated 15-PGDH levels in non-skeletal muscle tissues in subjects with age-related conditions or diseases, e.g., in the colon, brain, skin, spleen, or liver, leads to PGE2 and/or PGD2 degradation in these tissues and thus to lower levels of PGE2 and/or PGD2 and of PGE2 and/or PGD2 signaling, which has deleterious effects on tissue function that are manifested in aging. The present disclosure therefore provides compositions and methods based on the use of 15-PGDH activity as a therapeutic target in non-skeletal muscle tissues in subjects with age-related diseases or conditions. Inhibiting 15-PGDH in these tissues may restore or increase PGE2 and/or PGD2 levels in the tissues and may ameliorate their function, health, and/or physiological activity. Reducing 15-PGDH can thus lead to improved quality of life and outcomes for age-related diseases.

A non-limiting list of non-skeletal muscle tissues that can be treated using the present methods and compositions include, for example, epidermal, vascular, cardiac muscle, brain, bone, cartilage, smooth muscle, brown fat, spleen, liver, and the like. 15-PGDH elevation may occur in diseases of aged tissues including cardiovascular diseases (e.g., atrial fibrillation, stroke, ischemic heart diseases, cardiomyopathies, endocarditis, intracerebral hemorrhage), chronic respiratory diseases (e.g., chronic obstructive pulmonary disease, asbestosis, silicosis), nutritional diseases (trachoma, diarrheal diseases, encephalitis), kidney diseases (e.g., chronic kidney diseases), gastrointestinal and digestive diseases (e.g., NASH, pancreatitis, ulcer, intestinal obstruction), neurological disorders (e.g., Alzheimer's, dementia, Parkinson's), sensory disorders (e.g., hearing loss, macular degeneration, glaucoma), skin and subcutaneous diseases (e.g., cellulitis, ulcer, fungal skin diseases, pyoderma), osteoporosis, osteoarthritis, rheumatoid arthritis and the like. In addition, genetic disorders of these tissues that lead to premature aging syndromes, such as Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, and Rothmund-Thomson syndrome, as well as immunodeficiencies of these tissues that lead to premature aging syndromes, such as Ataxia telangiectasia, and infectious diseases of these tissues that lead to premature aging syndromes, such as human immunodeficiency virus (HIV), can also benefit from 15-PGDH inhibition.

Treating non-skeletal muscle tissues with inhibitors of 15-PGDH may provide numerous advantages, such as that the treatment can be localized to specific cell types that express elevated levels of the enzyme (e.g., diseased or aged non-skeletal muscle tissues), that it provides the ability to restore endogenous levels of PGE2 and/or PGD2 to achieve physiological “youthful” levels of PGE2 and/or PGD2, that it can target non-skeletal muscle tissues with high senescent cell infiltration (e.g., colon, skin, spleen), which is thought to have detrimental effects in aging and aging-associated conditions, and that it provides the possibility of targeting 15-PGDH with molecules with relatively long half-lives or by using gene therapy, in order to provide sustained, systemic PGE2 and/or PGD2 benefits.

### 2. General

Practicing the methods disclosed herein utilizes routine techniques in the field of molecular biology. Basic texts disclosing the general methods of use described herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“Age-related condition” or “age-related disease” refers to any disease, condition, or disorder that shows or potentially shows any signs or features associated with increasing age or passage of time in non-skeletal muscle tissues, including, e.g., loss or decrease of tissue function, loss or decrease of tissue health, loss or decrease of one or more physiological activities of the tissue, decreased protein synthesis in cells of the tissue, increased protein degradation in cells of the tissue, decreased survival or viability of the tissue, decreased proliferation of cells within the tissue, shortened telomeres in cells of the tissue, mitochondrial dysfunction in cells of the tissue, increased presence of senescent cells in the tissue, decreased levels of PGE2 and/or PGD2 in the tissue, etc. The condition or disease can be a result of natural aging processes due to the passage of time, of other factors such as lifestyle factors or disease, e.g., infectious disease, or of genetic conditions that cause premature aging.

A “non-skeletal muscle” tissue as used herein can refer to any tissue in the body other than skeletal muscle (e.g., other than musculi pectoralis complex, latissimus dorsi, teres major and subscapularis, brachioradialis, biceps, brachialis, pronator quadratus, pronator teres, flexor carpi radialis, flexor carpi ulnaris, flexor digitorum superficialis, flexor digitorum profundus, flexor pollicis brevis, opponens pollicis, adductor pollicis, flexor pollicis brevis, iliopsoas, psoas, rectus abdominis, rectus femoris, gluteus maximus, gluteus medius, medial hamstrings, gastrocnemius, lateral hamstring, quadriceps mechanism, adductor longus, adductor brevis, adductor magnus, gastrocnemius medial, gastrocnemius lateral, soleus, tibialis posterior, tibialis anterior, flexor digitorum longus, flexor digitorum brevis, flexor hallucis longus, extensor hallucis longus, ocular muscles, pharyngeal muscles, sphincter muscles, hand muscles, arm muscles, foot muscles, leg muscles, chest muscles, stomach muscles, back muscles, buttock muscles, shoulder muscles, head and neck muscles), and can encompass organs comprising multiple tissue types, as well as particular cell types within an organ or tissue. For example, a “non-skeletal muscle tissue” can include any of the following: epithelial tissue, nerve tissue, connective tissue, smooth muscle, cardiac muscle, epidermal tissue, vascular tissue, heart, kidney, brain, bone, cartilage, brown fat, spleen, liver, colon, sensory organs, thyroid, lung, blood, small intestine, dental tissue, ovaries or other reproductive tissue or organs, hair, cochlea, oligodendrocytes, and combinations thereof.

“Sarcopenia” refers to a loss of muscle mass, strength, and/or physical performance in association with age. Sarcopenia is a progressive process that can occur at different rates in different individuals and there is no minimum age for a diagnosis. For example, a human can be considered to have sarcopenia for the purposes of the methods provided herein if they are at least, e.g., 20, 25, 30, 35, 40, 45, 50, 55, 60, 65, 70, 75 years old or older.

“Aged muscle” or “aging muscle” refers to any muscle (e.g., skeletal muscle) that shows or potentially shows any signs or features associated with increasing age or passage of time in developed muscle, including, e.g., loss of muscle mass or strength, decreased protein synthesis, accumulation of intra- and extra-myocellular lipids, mitochondrial dysfunction, expression of atrogenes (e.g., Atrogin], Murf and MuSA), increased presence of senescent cells, increased levels of PGE2 metabolites (e.g., PGEM), etc. In some embodiments, aged or aging muscle refers to muscles in a subject with sarcopenia.

“Muscle atrophy” or “atrophic muscle” refers to any loss or wasting of muscle tissue, e.g., any amount of decrease of muscle size, mass, or function, for any reason, e.g., in relation to a condition such as sarcopenia, diabetes, muscular dystrophy, sarcopenic obesity, neuropathy, cancer cachexia, or HIV cachexia, frailty, or muscle atrophy resulting from immobilization or disuse.

The terms “prostaglandin E2”, “PGE2”, and “dinoprostone” refer to prostaglandin that can be synthesized from arachidonic acid via cyclooxygenase (COX) enzymes and terminal prosiaghndin E synthases (PGES). PGE2 plays a role in a number of biological functions including vasodilation, inflammation, and modulation of sleep/wake cycles. Structural and functional information about PGE2 can be found, e.g., in the entry for “Dinoprostone” of PubChem: pubchem.ncbi.nlm.nih.gov/compound/Dinoprostone, the contents of which are herein incorporated by reference in their entirety.

The term “prostaglandin D2” or “PGD2” refers to prostaglandin that can be synthesized from arachidonic acid via cyclooxygenase (COX) enzymes and PGD2 synthases (PTDS). PGD2 is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. Structural and functional information about PGD2 can be found, e.g., in the entry for “Prostaglandin D2” of PubChem: pubchem.ncbi.nlm.nih.gov/compound/448457, the contents of which are herein incorporated by reference in their entirety.

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos. NP_000851.2, NP_001139288.1, NP_001243236.1, NP_001243234.1, NP_001243235.1, NP_001350503.1, NP_001243230.1) can be targeted, as can any isoform with 50%, 60%, 70%, 80%, 85%, 90%, 95%, or higher identity to the amino acid sequences of any of GenBank Accession Nos. NP 000851.2, NP_001139288.1, NP_001243236.1, NP_001243234.1, NP 001243235.1, NP 001350503.1, NP_001243230.1, or of any other 15-PGDH enzyme.

A “15-PGDH inhibitor” refers to any agent that is capable of inhibiting, reducing, decreasing, attenuating, abolishing, eliminating, slowing, or counteracting in any way any aspect of the expression, stability, or activity of 15-PGDH. A 15-PGDH inhibitor can, for example, reduce any aspect of the expression, e.g., transcription, RNA processing, RNA stability, or translation of a gene encoding 15-PGDH, e.g., the human HPGD gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a 15-PGDH inhibitor can, for example, reduce the activity, e.g., enzymatic activity, of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor can, for example, reduce the stability of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “15-PGDH inhibitor”, also referred to herein as an “agent” or a “compound,” can be any molecule, either naturally occurring or synthetic, e.g., peptide, protein, oligopeptide (e.g., from about 5 to about 25 amino acids in length, e.g., about 5, 10, 15, 20, or 25 amino acids in length), small molecule (e.g., an organic molecule having a molecular weight of less than about 2500 daltons, e.g., less than 2000, less than 1000, or less than 500 daltons), antibody, nanobody, polysaccharide, lipid, fatty acid, inhibitory RNA (e.g., siRNA, shRNA, microRNA), modified RNA, polynucleotide, oligonucleotide, e.g., antisense oligonucleotide, aptamer, affimer, drug compound, or other compound.

A “senolytic agent” refers to any agent that is capable of inducing the death of senescent cells, e.g., inducing the death of at least 10%, 15%, 20%, 25%, 30%, 35%, 40%, 45%, 50 %, 55%, 60%, 65%, 70%, 75%, 80%, 85%, 90%, 95%, or more of a population of senescent cells, in vitro or in vivo. A non-limiting list of senolytic agents that can be used in the present methods include Bc12 inhibitors (e.g., navitoclax (ABT-263), ABT-737), pan-tyrosine kinase inhibitors (e.g., dasatinib), flavonoids (e.g., quercetin), peptides that interfere with the FOXO4-p53 interaction (e.g., FOXO4-DRI), a selective targeting system of senescent cells using galactooligosaccharide-coated nanoparticles, HSP90 inhibitors (e.g., 17-DMAG), and combinations thereof. In particular embodiments, a senolytic agent is capable of inducing the death of, e.g., 10%, 15%, 20%, 25%, 30%, 35%, 40%, 45%, 50%, 55%, 60%, 65%, 70%, 75%, 80%, 85%, 90%, 95% or more senescent cells, e.g., macrophages and/or fibroadipogenic progenitor (FAP) cells, within aged and/or atrophic muscle; and/or macrophages and/or fibroadipocytes within non-skeletal muscle tissue.

The terms “expression” and “expressed” refer to the production of a transcriptional and/or translational product, e.g., of a nucleic acid sequence encoding a protein (e.g., 15-PGDH). In some embodiments, the term refers to the production of a transcriptional and/or translational product encoded by a gene (e.g., the human HPGD gene) or a portion thereof. The level of expression of a DNA molecule in a cell may be assessed on the basis of either the amount of corresponding mRNA that is present within the cell or the amount of protein encoded by that DNA produced by the cell.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “V_{H}” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a 15-PGDH protein). Examples of antibody-binding fragments include, but are not limited to, a Fab fragment (a monovalent fragment consisting of the VL, VH, CL, and CHI domains), F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), V_{H} (heavy chain variable region), nanobodies, and any combination of those or any other functional portion of an immunoglobulin peptide capable of binding to target antigen (see, e.g., Fundamental Immunology (Paul ed., 4th ed. 2001).

The phrase “specifically binds” refers to a molecule (e.g., a 15-PGDH inhibitor such as a small molecule or antibody) that binds to a target with greater affinity, avidity, more readily, and/or with greater duration to that target in a sample than it binds to a non-target compound. In some embodiments, a molecule that specifically binds a target (e.g., 15-PGDH) binds to the target with at least 2-fold greater affinity than non-target compounds, e.g., at least 3-fold, 4-fold, 5-fold, 6-fold, 7-fold, 8-fold, 9-fold, 10-fold, 20-fold, 25-fold, 50-fold or greater affinity. For example, in some embodiments, a molecule that specifically binds to 15-PGDH will typically bind to 15-PGDH with at least a 2-fold greater affinity than to a non-15-PGDH target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer,” “administering,” or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. One skilled in the art will know of additional methods for administering a therapeutically effective amount of the compounds described herein for preventing or relieving one or more symptoms associated with a disease or condition.

The term “therapeutically effective amount” or “therapeutically effective dose” or “effective amount” refers to an amount of a compound (e.g., 15-PGDH inhibitor) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, ongoing disease process and type of treatment desired (e.g., aggressive vs. conventional treatment).

Therapeutically effective amounts of a pharmaceutical compound or composition, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutically acceptable carrier” refers to a carrier or a diluent that does not cause significant irritation to an organism and does not abrogate the biological activity and properties of the administered compound.

The terms “subject,” “individual,” and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “acute regimen”, in the context of administration of a compound, refers to a temporary or brief application of a compound to a subject, e.g., human subject, or to a repeated application of a compound to a subject, e.g., human subject, wherein a desired period of time (e.g., 1 day) lapses between applications. In some embodiments, an acute regimen includes an acute exposure (e.g., a single dose) of a compound to a subject over the course of treatment or over an extended period of time. In other embodiments, an acute regimen includes intermittent exposure (e.g., repeated doses) of a compound to a subject in which a desired period of time lapses between each exposure.

The term “chronic regimen,” in the context of administration of a compound, refers to a repeated, chronic application of a compound to a subject, e.g., human subject, over an extended period of time such that the amount or level of the compound is substantially constant over a selected time period. In some embodiments, a chronic regimen includes a continuous exposure of a compound to a subject over an extended period of time.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency, e.g., as described in Dar et al. (2016) Scientific Reports 6: article no. 20031 (2016), and as presented in the database accessible at crdd.osdd.net/servers/simamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

4. Methods of Enhancing Muscle Mass, Endurance, Strength, or Function in Atrophic and/or Aged Muscles

In one embodiment, provided herein are methods of enhancing a muscle function of an aged skeletal muscle in a subject, the method comprising: administering to the aged skeletal muscle a 15-PGDH inhibitor in an amount effective to inhibit 15-PGDH activity and/or to reduce 15-PGDH levels (e.g., mRNA and/or protein levels) in a senescent cell (e.g., present near or within the skeletal aged muscle, e.g., within the aged skeletal muscle microenvironment), thereby enhancing a muscle function of the aged skeletal muscle.

In another embodiment, provided herein are methods of increasing muscle mass, muscle strength, and/or muscle endurance of an aged skeletal muscle in a subject, the method comprising: administering to the aged skeletal muscle a 15-PGDH inhibitor in an amount effective to inhibit 15-PGDH activity and/or to reduce 15-PGDH levels (e.g., mRNA and/or protein levels) in a senescent cell (e.g., present near or within the aged skeletal muscle, e.g., within the aged skeletal muscle microenvironment), thereby increasing muscle mass, muscle strength, and/or muscle endurance of the aged skeletal muscle.

In another embodiment, a method of increasing a level of PGE2 in an aged skeletal muscle of a subject is provided, the method comprising: administering to the skeletal aged muscle (e.g., having a level of PGE2 that is reduced) a 15-PGDH inhibitor in an amount effective to increase PGE2 levels in the aged skeletal muscle (e.g., by inhibiting 15-PGDH activity or reducing 15-PGDH expression levels), thereby increasing a level of PGE2 in the aged skeletal muscle.

In another embodiment, a method of rejuvenating an aged skeletal muscle in a subject having one or more biomarkers of aging is provided, the method comprising: administering to the subject having one or more biomarkers of aging a 15-PGDH inhibitor in an amount effective to inhibit 15-PGDH activity and/or to reduce 15-PGDH levels (e.g., mRNA and/or protein levels) in the subject, thereby rejuvenating the aged skeletal muscle.

The methods provided herein may be used to enhance a function of aged skeletal muscle. The methods provided herein may be used to rejuvenate aged skeletal muscle. The methods provided herein may be used to increase muscle mass, muscle strength, muscle force, and/or muscle endurance of aged skeletal muscle.

In various aspects, the aged skeletal muscle may have one or more senescent cells (e.g., present within or near the skeletal muscle tissue). In some cases, the aged skeletal muscle may have a plurality of senescent cells (e.g., present within or near the skeletal muscle tissue). In some cases, the aged skeletal muscle may have an increased accumulation of senescent cells (e.g., within or near the skeletal muscle tissue) (e.g., relative to young skeletal muscle). In some cases, the aged skeletal muscle may have a number of senescent cells that is higher (e.g., substantially higher) than a number typically found in young skeletal muscle. The senescent cells may express one or more senescent markers. The senescent cells may have an increased level of one or more senescent markers relative to a non-senescent cell. The one or more senescent markers may be, without limitation, p15Ink4b, p16Ink4a, p19Arf, p21, Mmp13, I11a, I11b, and 116. In various aspects, the subject may be selected for treatment (e.g., by any method disclosed herein) based on a level of senescent cells present within skeletal muscle and/or based on the presence or levels of one or more senescent markers. In some cases, the presence of senescent cells within skeletal muscle (e.g., at a number higher than a number typically found in young muscle) and/or the presence and/or levels of one or more senescent markers may indicate that a treatment (e.g., any disclosed herein) is likely to provide a therapeutic benefit. In some cases, the senescent cells may express 15-PGDH (e.g., at levels effective to decrease a level of PGE2 within the aged skeletal muscle). In some cases, the senescent cells may be macrophages.

In various aspects, the subject may express one or more biomarkers of aging. A biomarker of aging may include, without limitation, an increase in 15-PGDH levels (e.g., relative to a level present in young skeletal muscle), a decrease in PGE2 levels (e.g., relative to a level present in young skeletal muscle), an increase in a PGE2 metabolite (e.g., relative to a level present in young skeletal muscle), an increase or a greater accumulation of senescent cells (e.g., relative to a level present in young skeletal muscle), an increase in expression of one or more atrogenes (e.g., Atrogin1 (MAFbx1), MuSA (Fbxo30), and Trim63 (MuRFI)) (e.g., relative to a level present in young skeletal muscle), a decrease in mitochondria biogenesis and/or function (e.g., relative to a level present in young skeletal muscle), and an increase in transforming growth factor pathway signaling (e.g., an increase in expression of one or more genes involved in a transforming growth factor signaling pathway, e.g., one or more of Activin receptor, Myostatin, a SMAD protein, and a bone morphogenetic protein) (e.g., relative to a level present in young skeletal muscle). In some cases, a biomarker of aging may include increased levels or activity of 15-PGDH (e.g., within the aged skeletal muscle) (e.g., relative to levels present in young skeletal muscle). In some cases, a biomarker of aging may include decreased levels of PGE2 (e.g., within the aged skeletal muscle) (e.g., relative to levels present in young skeletal muscle). In some cases, a biomarker of aging may include increased levels of a PGE2 metabolite (e.g., 15-keto PGE2 and 13,14-dihydro-15-keto PGE2) (e.g., relative to levels present in young skeletal muscle). In some cases, the presence of a biomarker of aging may indicate that the subject may benefit from treatment according to any method disclosed herein. In some cases, the subject is selected for treatment by a method disclosed herein (e.g., with a 15-PGDH inhibitor) based on the presence of one or more biomarkers of aging.

In various aspects, levels of PGE2 present within the aged skeletal muscle may be increased (e.g., after treatment with a 15-PGDH inhibitor, e.g., according to methods provided herein) relative to levels present in the aged skeletal muscle prior to the treatment (e.g., with the 15-PGDH inhibitor). PGE2 levels in the aged skeletal muscle may be increased (e.g., by any method disclosed herein) by at least 10% (e.g., at least 15%, at least 20%, at least 25%, at least 30%, at least 40%, at least 50%, at least 60%, at least 70%, or greater) relative to levels present in the aged skeletal muscle prior to the treatment (e.g., with the 15-PGDH inhibitor). In various aspects, levels of PGE2 present within the aged skeletal muscle may be increased (e.g., after treatment with a 15-PGDH inhibitor, e.g., according to methods provided herein) to a level substantially similar to a level present in young skeletal muscle. PGE2 levels in the aged skeletal muscle may be increased (e.g., by any method disclosed herein) to a level within about 50% or less of a level present in young skeletal muscle (e.g., within about 40%, within about 35%, within about 30%, within about 25%, within about 20%, within about 15%, within about 10%, within about 5%, or within about 1%).

In various aspects, levels of PGE2 metabolites present within the aged skeletal muscle may be decreased (e.g., after treatment with a 15-PGDH inhibitor, e.g., according to methods provided herein) relative to levels present in the aged skeletal muscle prior to the treatment (e.g., with the 15-PGDH inhibitor). PGE2 metabolite levels in the aged skeletal muscle may be decreased (e.g., by any method disclosed herein) by at least 10% (e.g., at least 15%, at least 20%, at least 25%, at least 30%, at least 40%, at least 50%, at least 60%, at least 70%, or greater) relative to levels present in the aged skeletal muscle prior to the treatment (e.g., with the 15-PGDH inhibitor). In various aspects, levels of PGE2 metabolites present within the aged skeletal muscle may be decreased (e.g., after treatment with a 15-PGDH inhibitor, e.g., according to methods provided herein) to a level substantially similar to a level present in young skeletal muscle. PGE2 metabolite levels in the aged skeletal muscle may be decreased (e.g., by any method disclosed herein) to a level within about 50% or less of a level present in young skeletal muscle (e.g., within about 40%, within about 35%, within about 30%, within about 25%, within about 20%, within about 15%, within about 10%, within about 5%, or within about 1%). The PGE2 metabolite may be 15-keto PGE2, 13,14-dihydro-15-keto PGE2, or both.

In some cases, treatment (e.g., with a 15-PGDH inhibitor, e.g., according to methods provided herein) may result in an increase in myofiber and/or myotube cross-sectional area and/or diameter (e.g., relative to the aged skeletal muscle prior to treatment, and/or increased to a level substantially similar (or within 50% or less) of a level of young skeletal muscle). In some cases, treatment (e.g., with a 15-PGDH inhibitor, e.g., according to methods provided herein) may result in an increase in cross-sectional area and/or diameter of oxidative (type IIa) and/or glycolytic (type IIb) fibers (e.g., relative to the aged skeletal muscle prior to treatment, and/or increased to a level substantially similar (or within about 50% or less) of a level of young skeletal muscle).

In some cases, treatment (e.g., with a 15-PGDH inhibitor, e.g., according to methods provided herein) may result in a decrease in expression levels (e.g., in the aged skeletal muscle) of one or more atrogenes selected from the group consisting of: Atrogin1 (MAFbx1), MuSA (Fbxo30), and Trim63 (MuRFI) (e.g., relative to the aged skeletal muscle prior to treatment, and/or increased to a level substantially similar (or within about 50% or less) of a level of young skeletal muscle). In some cases, treatment (e.g., with a 15-PGDH inhibitor, e.g., according to methods provided herein) may result in an increase in expression levels (e.g., in the aged skeletal muscle) of one or more components of a mitochondria complex (e.g., relative to the aged skeletal muscle prior to treatment, and/or increased to a level substantially similar (or within about 50% or less) of a level of young skeletal muscle).

The one or more components of a mitochondria complex may be selected from the group consisting of: Ndufa11, Ndufa12, Ndufa13, Ndufa2, Ndufa3, Ndufa4, Ndufa5, Ndufa10, Ndufb5, Ndufcl, Ndufs4, Ndufs8, Ndufv1, Ndufv2, Uqcrb, Uqcrcl, Uqcrh, Uqcrq, Ucqr10, Cox8b, Cox7al, Cox7a2, Cox7b, Cox6c, Cox5a, Cox5b, Atp5fl, Atp5gl, Atp5h, Atp5j2, Atp5o, Atp5e, and Atp5k. In some cases, treatment (e.g., with a 15-PGDH inhibitor, e.g., according to methods provided herein) may result in an increase of an expression level of peroxisome proliferator-activated receptor gamma coactivator 1-alpha (Pgc1α) (e.g., relative to the aged skeletal muscle prior to treatment, and/or increased to a level substantially similar (or within about 50% or less) of a level of young skeletal muscle). In some cases, treatment (e.g., with a 15-PGDH inhibitor, e.g., according to methods provided herein) may result in a decrease in expression levels of one or more genes selected from the group consisting of: Tnfaipl, Klhdc8a, Fbxw 11, Tnfaip3, Herc3, Herc2, Hdac4, Traf6, Ankibl, Mib1, Pja2, Ubr3, Thbsl, Smad3, Acvr2a, Rgmb, Tgfb2, and Mstn (e.g., relative to the aged skeletal muscle prior to treatment, and/or increased to a level substantially similar (or within about 50% or less) of a level of young skeletal muscle).

In various aspects, muscle function of the aged skeletal muscle may be enhanced (e.g., after treatment with a 15-PGDH inhibitor, e.g., according to methods provided herein) relative to the aged skeletal muscle prior to the treatment (e.g., with the 15-PGDH inhibitor). Muscle function of the aged skeletal muscle may be enhanced (e.g., by any method disclosed herein) by at least 10% (e.g., at least 15%, at least 20%, at least 25%, at least 30%, at least 40%, at least 50%, at least 60%, at least 70%, or greater) relative to the aged skeletal muscle prior to the treatment (e.g., with the 15-PGDH inhibitor). In various aspects, muscle function of the aged skeletal muscle may be enhanced (e.g., after treatment with a 15-PGDH inhibitor, e.g., according to methods provided herein) to a level substantially similar to a level present in young skeletal muscle. Muscle function of the aged skeletal muscle may be enhanced (e.g., by any method disclosed herein) to a level within about 50% or less of a level present in young skeletal muscle (e.g., within about 40%, within about 35%, within about 30%, within about 25%, within about 20%, within about 15%, within about 10%, within about 5%, or within about 1%). Muscle function may include increased protein synthesis, increased cell proliferation, increased cell survival, decreased protein degradation, or any combination thereof.

In various aspects, muscle mass, muscle strength, and/or muscle endurance of the aged skeletal muscle may be increased (e.g., after treatment with a 15-PGDH inhibitor, e.g., according to methods provided herein) relative to the aged skeletal muscle prior to the treatment (e.g., with the 15-PGDH inhibitor). Muscle mass, muscle strength, and/or muscle endurance of the aged skeletal muscle may be increased (e.g., by any method disclosed herein) by at least 10% (e.g., at least 15%, at least 20%, at least 25%, at least 30%, at least 40%, at least 50%, at least 60%, at least 70%, or greater) relative to the aged skeletal muscle prior to the treatment (e.g., with the 15-PGDH inhibitor). In various aspects, muscle mass, muscle strength, and/or muscle endurance of the aged skeletal muscle may be increased (e.g., after treatment with a 15-PGDH inhibitor, e.g., according to methods provided herein) to a level substantially similar young skeletal muscle. Muscle mass, muscle strength, and/or muscle endurance of the aged skeletal muscle may be increased (e.g., by any method disclosed herein) to a level within about 50% or less of young skeletal muscle (e.g., within about 40%, within about 35%, within about 30%, within about 25%, within about 20%, within about 15%, within about 10%, within about 5%, or within about 1%).

In further embodiments, the present disclosure provides a method of enhancing a function of a skeletal muscle in a subject, the method comprising: administering to the subject a 15-PGDH inhibitor in an amount effective to inhibit 15-PGDH activity and/or reduce 15-PGDH levels in the skeletal muscle, thereby enhancing a function of the skeletal muscle in the subject. In some cases, the skeletal muscle is healthy skeletal muscle. In some cases, the skeletal muscle is uninjured, has not or is not undergoing regeneration, and/or has not or is not undergoing significant or substantial exercise. In some cases, the skeletal muscle is not dystrophic, atrophic, or aged. In some cases, the method is independent of an increase in proliferation of muscle stem cells in the subject. In some cases, the skeletal muscle is young skeletal muscle. In some cases, the subject is less than 30 years of age (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 year of age). In various aspects, the method results in an increase in muscle mass, an increase in muscle strength, an increase in muscle endurance, or any combination thereof (e.g., relative to the skeletal muscle prior to the treatment, e.g., with the 15-PGDH inhibitor). In various aspects, the method results in an increase in protein synthesis, an increase in cell proliferation, an increase in cell survival, a decrease in protein degradation, or any combination thereof (e.g., relative to the skeletal muscle prior to the treatment, e.g., with the 15-PGDH inhibitor).

The present disclosure further provides methods of increasing the function of aged and/or atrophic muscle in a subject, e.g., a human subject, comprising administering a 15-PGDH inhibitor to the subject. The administration of the 15-PGDH inhibitor can be systemic or local, e.g., by intramuscular injection, and can enhance any of a number of aspects of the aged and/or atrophied muscle, including enhancing mass, function, strength, endurance, exercise performance, or any other measure of muscle function in the subject. In particular embodiments, the administration of the 15-PGDH inhibitor leads to an increase in the size of myofibers and/or myotubes in the aged and/or atrophied muscles in the subject, e.g., an increase in their diameter or cross-section. In other embodiments, the administration of the 15-PGDH inhibitor results in protection against muscle cell death in the subject, in particular in mature muscle cells.

In particular embodiments, the inhibition of 15-PGDH in the subject leads to an increase in PGE2, e.g., an elevation, increase or restoration of PGE2 levels, in the muscles of the subject and a decrease in PGE2 metabolites such as 15-keto-PGE2 or 13,14-dihydro-15-keto-PGE2 (PGEM). In some embodiments, the inhibition also leads to an increase in EP4 activity in the atrophied and/or aged muscles of the subject. In some embodiments, the inhibition also leads to a decrease in Atrogin1 levels or activity in the atrophied and/or aged muscles of the subject.

In particular embodiments, the herein-described benefits of 15-PGDH inhibitor administration, e.g., enhanced muscle strength, mass, exercise performance, endurance, myofiber or myotube size, etc., occur independently of any increase in the number or proliferation of muscle stem cells (MuSCs) in the atrophied and/or aged muscles of the subject. In other words, while there may be an increase in the number or proliferation of MuSCs in the subject, the herein-described effects do not require the MuSCs and would occur even without an increase in the number or proliferation of MuSCs. In particular embodiments, the aged and/or atrophic muscle is not injured nor has it undergone exercise or regeneration.

In some embodiments, the administration of the 15-PGDH inhibitor inhibits 15-PGDH activity or reduces 15-PGDH levels in senescent cells, e.g., macrophages and/or fibroadipogenic progenitor (FAP) cells, within the aged and/or atrophied muscle. In some embodiments, the methods further comprise the administration of a senolytic agent to the subject. Examples of senolytic agents that can be used include, inter alia, Bc12 inhibitors such as navitoclax (also known as ABT-263) and ABT-737, pan-tyrosine kinase inhibitors such as dasatinib together with a flavonoid such as quercetin, a peptide which interferes with the FOXO4-p53 interaction such as FOXO4-DRI, a selective targeting system of senescent cells using galactooligosaccharides-coated nanoparticles, combination therapy comprising dasatinib and quercetin, and HSP90 inhibitors such as 17-DMAG. It will be appreciated that the senolytic agent can be administered together with the 15-PGDH inhibitor, e.g., within a single pharmaceutical formulation, or separately.

### Subjects

The subject can be any subject, e.g., a human or other mammal, with aged and/or atrophic skeletal muscle, or at risk of having aged and/or atrophic skeletal muscle. In some embodiments, the subject is a human. In some embodiments, the subject is an adult (e.g., an adult with age-related sarcopenia). In some embodiments, the subject is a child (e.g., a child with a muscular dystrophy such as Duchenne muscular dystrophy). In some embodiments, the subject is female (e.g., an adult female). In some embodiments, the subject is male (e.g., an adult male).

In some embodiments, the subject is human, and the method further comprises a step in which the human is selected for treatment with the 15-PGDH inhibitor based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has sarcopenia or aged muscle. In some embodiments, the subject is determined to have aged and/or atrophic muscle as determined using any method of assessing muscle strength or function, e.g., grip test, walk speed, muscle power test, functional tests, resistance tests, or treadmill, by imaging-based tests, by assessment of muscle mass, and/or by molecular or cellular analysis in, e.g., a muscle biopsy taken from the subject by a physician or other qualified medical professional.

In some embodiments, the subject has a condition or disease associated with muscle atrophy such as diabetes, frailty, muscular dystrophy, sarcopenic obesity, neuropathy, cachexia such as cancer cachexia or HIV cachexia, or has muscle atrophy due to immobilization or muscle disuse. In some embodiments, the subject has a muscular dystrophy selected from the group consisting of Duchenne muscular dystrophy (DMD), Becker muscular dystrophy, congenital muscular dystrophy, distal muscular dystrophy, Emery-Dreifuss muscular dystrophy, facioscapulohumeral muscular dystrophy, limb girdle muscular dystrophy, myotonic muscular dystrophy (MDD), and oculopharyngeal muscular dystrophy. In particular embodiments, the muscular dystrophy is Duchenne muscular dystrophy.

In particular embodiments, the muscle is skeletal muscle. In some embodiments, the muscle is uninjured and/or has not undergone exercise or regeneration. The muscle can be any muscle of the body including, but not limited to, musculi pectoralis complex, latissimus dorsi, teres major and subscapularis, brachioradialis, biceps, brachialis, pronator quadratus, pronator teres, flexor carpi radialis, flexor carpi ulnaris, flexor digitorum superficialis, flexor digitorum profundus, flexor pollicis brevis, opponens pollicis, adductor pollicis, flexor pollicis brevis, iliopsoas, psoas, rectus abdominis, rectus femoris, gluteus maximus, gluteus medius, medial hamstrings, gastroenemius, lateral hamstring, quadriceps mechanism, adductor longus, adductor brevis, adductor magnus, gastroenemius medial, gastroenemius lateral, soleus, tibialis posterior, tibialis anterior, flexor digitorum longus, flexor digitorum brevis, flexor hallucis longus, extensor hallucis longus, ocular muscles, pharyngeal muscles, sphincter muscles, hand muscles, arm muscles, foot muscles, leg muscles, chest muscles, stomach muscles, back muscles, buttock muscles, shoulder muscles, head and neck muscles, and the like.

In some embodiments, subjects are identified for treatment based on a diagnosis of a condition or disease associated with muscle atrophy; based on a determination of the presence of or potential for muscle atrophy; based on a subject's age, e.g., an age associated with sarcopenia or of a potential for sarcopenia, or based on a detection of any of the herein-described features of aged and/or atrophic muscle. For example, a detection in muscles of elevated levels of PGE2 metabolites, e.g., 15-keto-PGE2 or PGEM, of decreased protein synthesis in muscles, of decreased myofiber and/or myotube size, of decreased muscle mass, of decreased muscle strength, function or endurance, of increased levels or activity of Atrogin1, of decreased activity of EP4, of elevated expression of genes associated with the senescence phenotype such as Ptges, Cox2, of elevated numbers of senescent cells, of the presence of one or more senescent markers, of elevated levels or activity of 15-PGDH, in particular in senescent cells, e.g., macrophages and/or fibroadipogenic progenitor cells, can indicate that the subject is a candidate for treatment with a 15-PGDH inhibitor. In particular embodiments, such a detection is made where the muscle has not been injured nor undergone exercise or regeneration.

The assessment of muscle function, strength, endurance, mass, or of any of the herein-described features in a subject can be assessed using any of a wide variety of methods known to those of skill in the art, e.g., by analysis of muscle performance such as by grip test, walk speed, muscle power test, functional tests, resistance tests, or treadmill, by imaging-based tests, by assessment of muscle mass, and/or by molecular or cellular analysis in, e.g., a muscle biopsy taken from the subject.

In some embodiments, the subject is a farm animal, e.g., livestock for human consumption, such as a porcine, bovine, ovine, poultry, or fish, and the methods are used, e.g., to enhance muscle mass, function, or strength in an aging animal, e.g., an animal with aged and/or atrophic muscle. In some such embodiments, the animal is administered a small molecule inhibitor of 15-PGDH. In some embodiments, a vector or expression cassette comprising a nucleic acid inhibitor of 15-PGDH, e.g., an shRNA, is introduced into the animal such that the nucleic acid inhibitor is expressed in the cells of the animal, e.g., the muscle cells. In some embodiments, a vector or expression cassette comprising a polynucleotide encoding a polypeptide inhibitor of 15-PGDH, e.g., an antibody or peptide, is introduced into the animal such that the polypeptide inhibitor is expressed in the cells of the animal, e.g., the muscle cells. In some embodiments, gene therapy is used, e.g., such that all or part of an endogenous 15-PGDH encoding gene is replaced with a form of the gene that is less active, less stable, or less highly expressed in cells, e.g., muscle cells, of the animal. In some embodiments, modified RNA, e.g., a chemically modified RNA inhibitor such as shRNA or a chemically modified mRNA encoding a polypeptide 15-PGDH inhibitor is introduced into the animal such that the RNA inhibitor or expressed protein inhibitor is present in muscle cells of the animal.

5. Methods of Enhancing Tissue Function in Subjects with Age-Related Conditions

In another embodiment, a method is provided for rejuvenating an aged non-skeletal muscle tissue in a subject, the method comprising: administering to the subject an amount of a 15-PGDH inhibitor effective to inhibit 15-PGDH, thereby rejuvenating the aged non-skeletal muscle tissue.

In various aspects, the aged non-skeletal muscle tissue may have one or more senescent cells (e.g., present within or near the aged tissue). In some cases, the aged non-skeletal muscle tissue may have a plurality of senescent cells (e.g., present within or near the aged tissue). In some cases, the aged non-skeletal muscle tissue may have an increased accumulation of senescent cells (e.g., within or near the aged non-skeletal muscle tissue) (e.g., relative to young non-skeletal muscle tissue). In some cases, the aged non-skeletal muscle tissue may have a number of senescent cells that is higher (e.g., substantially higher) than a number typically found in young non-skeletal muscle tissue. The senescent cells may express one or more senescent markers. The senescent cells may have an increased level of one or more senescent markers relative to a non-senescent cell. The one or more senescent markers may be, without limitation, p15Ink4b, p16Ink4a, p19Arf, p21, Mmp13, I11a, I11b, and 116. In various aspects, the subject may be selected for treatment (e.g., by any method disclosed herein) based on a level of senescent cells present within the aged non-skeletal muscle tissue and/or based on the presence or levels of one or more senescent markers. In some cases, the presence of senescent cells within the aged non-skeletal muscle tissue (e.g., at a number higher than a number typically found in young non-skeletal muscle tissue) and/or the presence and/or levels of one or more senescent markers may indicate that a treatment (e.g., any disclosed herein) is likely to provide a therapeutic benefit. In some cases, the senescent cells may express 15-PGDH (e.g., at levels effective to decrease a level of PGE2 within the aged non-skeletal muscle tissue). In some cases, the senescent cells may be macrophages.

In various aspects, the subject may express one or more biomarkers of aging. A biomarker of aging may include, without limitation, an increase in 15-PGDH levels (e.g., relative to a level present in young non-skeletal muscle tissue), a decrease in PGE2 levels (e.g., relative to a level present in young non-skeletal muscle tissue), an increase in a PGE2 metabolite (e.g., relative to a level present in young non-skeletal muscle tissue), an increase or a greater accumulation of senescent cells (e.g., relative to a level present in young non-skeletal muscle tissue), an increase in expression of one or more atrogenes (e.g., Atrogin1 (MAFbx1), MuSA (Fbxo30), and Trim63 (MuRF1)) (e.g., relative to a level present in young non-skeletal muscle tissue), a decrease in mitochondria biogenesis and/or function (e.g., relative to a level present in young non-skeletal muscle tissue), and an increase in transforming growth factor pathway signaling (e.g., an increase in expression of one or more genes involved in a transforming growth factor signaling pathway, e.g., one or more of Activin receptor, Myostatin, a SMAD protein, and a bone morphogenetic protein) (e.g., relative to a level present in young non-skeletal muscle tissue). In some cases, a biomarker of aging may include increased levels or activity of 15-PGDH (e.g., within the aged non-skeletal muscle tissue) (e.g., relative to a level present in young non-skeletal muscle tissue). In some cases, a biomarker of aging may include decreased levels of PGE2 (e.g., within the aged non-skeletal muscle tissue) (e.g., relative to a level present in young non-skeletal muscle tissue). In some cases, a biomarker of aging may include increased levels of a PGE2 metabolite (e.g., 15-keto PGE2 and 13,14-dihydro-15-keto PGE2, e.g., within the aged non-skeletal muscle tissue) (e.g., relative to a level present in young non-skeletal muscle tissue). In some cases, the presence of a biomarker of aging may indicate that the subject is likely to benefit from treatment according to any method disclosed herein. Young non-skeletal muscle may include non-skeletal muscle from a subject under the age of 30 (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 years of age).

In various aspects, levels of PGE2 present within the aged non-skeletal muscle tissue may be increased (e.g., after treatment with a 15-PGDH inhibitor, e.g., according to methods provided herein) relative to levels present in the aged non-skeletal muscle tissue prior to the treatment (e.g., with the 15-PGDH inhibitor). PGE2 levels in the aged non-skeletal muscle tissue may be increased (e.g., by any method disclosed herein) by at least 10% (e.g., at least 15%, at least 20%, at least 25%, at least 30%, at least 40%, at least 50%, at least 60%, at least 70%, or greater) relative to levels present in the aged non-skeletal muscle tissue prior to the treatment (e.g., with the 15-PGDH inhibitor). In various aspects, levels of PGE2 present within the aged non-skeletal muscle tissue may be increased (e.g., after treatment with a 15-PGDH inhibitor, e.g., according to methods provided herein) to a level substantially similar to a level present in young non-skeletal muscle tissue. PGE2 levels in the aged non-skeletal muscle tissue may be increased (e.g., by any method disclosed herein) to a level within about 50% or less of a level present in young non-skeletal muscle tissue (e.g., within about 40%, within about 35%, within about 30%, within about 25%, within about 20%, within about 15%, within about 10%, within about 5%, or within about 1%).

In various aspects, levels of PGE2 metabolites present within the aged non-skeletal muscle tissue may be decreased (e.g., after treatment with a 15-PGDH inhibitor, e.g., according to methods provided herein) relative to levels present in the aged non-skeletal muscle tissue prior to the treatment (e.g., with the 15-PGDH inhibitor). PGE2 metabolite levels in the aged non-skeletal muscle tissue may be decreased (e.g., by any method disclosed herein) by at least 10% (e.g., at least 15%, at least 20%, at least 25%, at least 30%, at least 40%, at least 50%, at least 60%, at least 70%, or greater) relative to levels present in the aged non-skeletal muscle tissue prior to the treatment (e.g., with the 15-PGDH inhibitor). In various aspects, levels of PGE2 metabolites present within the aged non-skeletal muscle tissue may be decreased (e.g., after treatment with a 15-PGDH inhibitor, e.g., according to methods provided herein) to a level substantially similar to a level present in young non-skeletal muscle tissue. PGE2 metabolite levels in the aged non-skeletal muscle tissue may be decreased (e.g., by any method disclosed herein) to a level within about 50% or less of a level present in young non-skeletal muscle tissue (e.g., within about 40%, within about 35%, within about 30%, within about 25%, within about 20%, within about 15%, within about 10%, within about 5%, or within about 1%). The PGE2 metabolite may be 15-keto PGE2, 13,14-dihydro-15-keto PGE2, or both. The PGE2 metabolite may be 15-keto PGE2, 13,14-dihydro-15-keto PGE2, or both.

In various aspects, a function of the aged non-skeletal muscle tissue may be enhanced (e.g., after treatment with a 15-PGDH inhibitor, e.g., according to methods provided herein) relative to the aged non-skeletal muscle tissue prior to the treatment (e.g., with the 15-PGDH inhibitor). A function of the aged non-skeletal muscle tissue may be enhanced (e.g., by any method disclosed herein) by at least 10% (e.g., at least 15%, at least 20%, at least 25%, at least 30%, at least 40%, at least 50%, at least 60%, at least 70%, or greater) relative to levels present in the aged non-skeletal muscle tissue prior to the treatment (e.g., with the 15-PGDH inhibitor). In various aspects, a function of the aged non-skeletal muscle tissue may be enhanced (e.g., after treatment with a 15-PGDH inhibitor, e.g., according to methods provided herein) to a level substantially similar to a level present in young non-skeletal muscle tissue. A function of the aged non-skeletal muscle tissue may be enhanced (e.g., by any method disclosed herein) to a level within about 50% or less of a level present in young non-skeletal muscle tissue (e.g., within about 40%, within about 35%, within about 30%, within about 25%, within about 20%, within about 15%, within about 10%, within about 5%, within about 1%). A function may include increased protein synthesis, increased cell proliferation, increased cell survival, decreased protein degradation, or any combination thereof.

In some instances, treatment (e.g., with a 15-PGDH inhibitor, e.g., according to methods provided herein) may result in rejuvenation of the aged non-skeletal muscle tissue (e.g., an increase in one or more functions of the aged non-skeletal muscle tissue).

The present disclosure provides methods of increasing the function, health, and other properties of non-skeletal muscle tissues in subjects, e.g., human subjects, with an age-related condition or disease, comprising administering a 15-PGDH inhibitor to the subject. The administration of the 15-PGDH inhibitor can be systemic or local, and can enhance any of a number of aspects of the tissue including enhancing function, physiological activity, endurance, performance on any assay for assessing tissue function, or any other measure of tissue function or health in the subject. In some embodiments, the administration of the 15-PGDH inhibitor results in protection against cell death in the non-skeletal muscle tissue in the subject. In some embodiments, the administration of the 15-PGDH inhibitor results in reduced protein degradation in the non-skeletal muscle tissue in the subject. In some embodiments, the administration of the 15-PGDH inhibitor results in increased protein synthesis in the non-skeletal muscle tissue in the subject. In some embodiments, administration of the 15-PGDH inhibitor may result in increased endurance (e.g., during exercise, e.g., as measured on a treadmill). In some cases, the increased endurance of the subject (e.g., during exercise) may be due to an increased function and/or rejuvenation of the aged non-skeletal muscle tissue (e.g., heart, lungs, bones, etc.).

The present disclosure also provides methods of measuring 15-PGDH levels in non-skeletal muscle tissues of a subject with an age-related condition. Such methods are useful, e.g., for the use of 15-PGDH as a biomarker of aging or aging non-skeletal muscle tissues and/or for a loss or decrease of function of non-skeletal muscle tissues, e.g., wherein an elevated level of 15-PDGH levels or activity, e.g., an increase of 10%, 20%, 30%, 40%, 50%, 60%, 70%, 80%, 90%, 100% or more relative to a control level in a subject without an age-related condition is indicative of aging or a loss or decrease of function in the tissue. In such methods, 15-PGDH can be assessed in any of a number of ways, e.g., by detecting levels of a transcript encoding a 15-PGDH protein, by detecting levels of a 15-PGDH polypeptide, or by detecting 15-PGDH enzymatic activity.

In particular embodiments, the inhibition of 15-PGDH in the subject leads to an increase in PGE2 and/or PGD2, e.g., an elevation, increase, or restoration of PGE2 and/or PGD2 levels, in the non-skeletal muscle tissue of the subject, and a decrease in PGE2 and/or PGD2 metabolites such as 15-keto-PGE2, 13,14-dihydro-15-keto-PGE2 (PGEM), 15-keto-PGD2, and 13,14-Dihydro-15-keto-PGD2. In some embodiments, the inhibition also leads to increased signaling through PGE2 receptors, e.g., EP1, EP2, EP3, and/or EP4 (also known as Ptger1, Ptger2, Ptger3, Ptger4) in the non-skeletal muscle tissue. In some embodiments, the inhibition also leads to increased signaling through PGD2 receptors, e.g., DP1 and/or DP2 (also known as PTGDR1, PTGDR2/CRTH2).

In particular embodiments, the herein-described benefits of 15-PGDH inhibitor administration in the non-skeletal muscle tissue, e.g., enhanced tissue health, function, physiological activity, etc., occur independently of any regeneration of the tissue in the subject. In other words, while there may be regeneration of the tissue in the subject, e.g., if the tissue has been injured or damaged, the herein-described effects do not require the regeneration and would occur even without the regeneration. In particular embodiments, the non-skeletal muscle tissue is not injured or damaged and has not or does not undergo regeneration.

In some embodiments, the administration of the 15-PGDH inhibitor inhibits 15-PGDH activity or reduced 15-PGDH levels in senescent cells, e.g., macrophages, fibroadipocytes, other mononuclear interstitial tissue resident cells including other immune cells, fibroblasts, endothelial cells, preadipocytes, and/or adipocytes, within the non-skeletal muscle tissue of the subject. In some embodiments, the methods further comprise the administration of a senolytic agent to the subject. Examples of senolytic agents that can be used include, inter alia, Bc12 inhibitors such as navitoclax (also known as ABT-263) and ABT-737, pan-tyrosine kinase inhibitors such as dasatinib together with a flavonoid such as quercetin, a peptide which interferes with the FOXO4-p53 interaction such as FOXO4-DRI, a selective targeting system of senescent cells using galactooligosaccharides-coated nanoparticles, a combination drug therapy comprising dasatinib and quercetin, and HSP90 inhibitors such as 17-DMAG. It will be appreciated that the senolytic agent can be administered together with the 15-PGDH inhibitor, e.g., within a single pharmaceutical formulation, or separately.

### Subjects

The subject can be any subject, e.g., a human or other mammal, with an age-related condition or at risk of having an age-related condition. In some embodiments, the subject is a human. In some embodiments, the subject is an adult. In some embodiments, the subject is a child (e.g., a child with progeria). In some embodiments, the subject is female (e.g., an adult female). In some embodiments, the subject is male (e.g., an adult male).

In some embodiments, the subject is human, and the method further comprises a step in which the human is selected for treatment with the 15-PGDH inhibitor based on a diagnosis of an age-related condition or disease, or on the potential for or risk of developing an age-related condition or disease. In some such embodiments, the human is selected based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has an age-related condition or disease. In some embodiments, the human is selected based on a potential for an age-related condition or disease, based on the presence or potential presence of an environmental, lifestyle, or medical factor linked to premature aging of one or more non-skeletal muscle tissues, such as smoking, drinking, diet, lack of physical activity, insufficient sleep, drug use, exposure to UV rays, exposure to extreme temperatures, stress, excess weight, or health-related factors such as infections, mental illness, cancer, diabetes, etc. In some embodiments, the subject has an age-related condition caused by premature aging of one or more tissues, e.g., a genetic disorder such as Osteogenesis imperfecta, Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, Rothmund-Thomson syndrome, an immunodeficiency of these tissues that lead to premature aging syndromes, such as Ataxia telangiectasia, or an infectious disease of these tissues that lead to premature aging syndromes, such as human immunodeficiency virus (HIV).

In some embodiments, the subject is determined to have aged tissues or have an age-related condition or disease as determined using any method of assessing any measure of the function, performance, health, strength, endurance, physiological activity, or any other property of a non-skeletal muscle tissue, e.g., a performance-based, imaging-based, physiological, molecular, cellular, or functional assay. For example, a heart can be assessed using any method of assessing heart function or health, such as angiograms, electrocardiograms, treadmill test, echocardiogram, etc. In some embodiments, the subject is selected for treatment based on a detection of elevated levels of 15-PGDH transcript, protein, or enzymatic activity in a non-skeletal muscle related tissue, or on a detection of decreased levels of PGE2 and/or PGD2 in the tissue.

In some embodiments, the methods comprise an additional step subsequent to the administration of a 15-PGDH inhibitor, comprising assessing the health, function, performance, or any other property of a non-skeletal muscle tissue in the subject, or comprising assessing the level of 15-PGDH (e.g., of 15-PGDH protein, transcript, or activity) and/or PGE2 and/or PGD2 in the non-skeletal muscle tissue in the subject, e.g., to ascertain the potential effects of the prior administration of the 15-PGDH inhibitor on the tissue. In some such embodiments, the health, function, performance, 15-PGDH level, PGE2 level, PGD2 level, or other property of the tissue is detected or examined and compared to the health, function, performance, 15-PGDH level, PGE2 level, PGD2 level, or other property of the tissue prior to the administration of the 15-PGDH inhibitor or to a control value, wherein a determination that the health, function, or performance of the tissue has improved, that the 15-PGDH level has decreased, that the PGE2 level and/or PGD2 level has increased, in the tissue subsequent to the administration of the inhibitor as compared to the value obtained prior to the administration of the 15-PGDH inhibitor or relative to a control value, indicates that the 15-PGDH inhibitor has had a beneficial effect in the non-skeletal muscle tissue of the subject.

In some embodiments, the subject has an age-related condition, disorder or disease such as a cardiovascular disease or condition (e.g., atrial fibrillation, stroke, ischemic heart diseases, cardiomyopathies, endocarditis, intracerebral haemorrhage, hypertension), a chronic respiratory disease or condition (e.g., chronic obstructive pulmonary disease, asbestosis, silicosis), a nutritional disease or condition (e.g., trachoma, diarrheal diseases, encephalitis), a kidney disease or condition (e.g., chronic kidney diseases), a gastrointestinal or digestive disease or condition (e.g., NASH, pancreatitis, ulcer, intestinal obstruction), a neurological disorder (e.g., Alzheimer's, dementia, Parkinson's, cognitive decline), a sensory disorder (e.g., hearing loss, vision loss, loss of sense of smell or sense of taste, macular degeneration, retinitis pigmentosa, glaucoma), a skin or subcutaneous disease or condition (e.g., cellulitis, ulcer, fungal skin diseases, pyoderma), osteoporosis, osteoarthritis, rheumatoid arthritis, a genetic disease causing premature aging in one or more non-skeletal muscle tissues (e.g., progeria, osteogenesis imperfecta, Bloom syndrome, Cockayne Syndrome, Hutchinson-Gilford Progeria Syndrome, Mandibuloacral Dysplasia, Progeroid Syndrome, Rothmund-Thomson Syndrome, Seip Syndrome, Werner Syndrome, Down Syndrome, Acrogeria, Rothmund-Thomson syndrome), an immunodeficiency of these tissues that lead to premature aging syndromes (e.g., Ataxia telangiectasia), or an infectious disease of these tissues that leads to premature aging syndromes, (e.g., human immunodeficiency virus (HIV)), and the like.

The administration of the 15-PGDH inhibitor can provide improvement in any of these conditions, and can help improve, e.g., osteoporosis, hair loss, aged skin, cognitive disorders, sensory disorders, aged hematopoietic stem cell function, and gastrointestinal function.

The present methods and compositions can be used to treat any non-skeletal muscle tissue, or organs including such tissues, or cells within such tissues, including epithelial tissue, nerve tissue, connective tissue, smooth muscle, cardiac muscle, epidermal tissues, vascular tissues, heart, kidney, brain, bone, cartilage, brown fat, spleen, liver, colon, sensory organs, thyroid, lung, blood, small intestine, dental tissue, ovaries or other reproductive tissue, hair, cochlea, oligodendrocytes, etc.

In some embodiments, subjects are identified for treatment based on a diagnosis of an age-related condition, disorder, or disease; based on a determination of the presence of or potential for age-related loss of non-skeletal muscle tissue function, health, or performance; based on a subject's age, e.g., an age associated with an age-related condition or disease; or based on a detection of any of the herein-described features of aged non-skeletal muscle tissues, e.g., of elevated levels of PGE2 and/or PGD2 metabolites such as 15-keto-PGE2, PGEM, 15-keto-PGD2, or 13,14-Dihydro-15-PGD2, of decreased levels of PGE2 and/or PGD2, of decreased protein synthesis, of decreased mitochondrial activity, of decreased signaling through the EP1, EP2, EP3, EP4, DP1, and/or DP2 receptors, of elevated expression of genes associated with the senescence phenotype such as p16 (Ink4a) or p21 (Cdkn1a), of shortened telomere length in cells of the tissue, of elevated numbers of senescent cells in a non-skeletal muscle tissue, or of elevated levels or activity of 15-PGDH, in particular in senescent cells, e.g., macrophages, fibroadipocytes, fibroblasts, endothelial cells, etc.

In some embodiments, the subject is a pet or a farm animal such as a porcine, bovine, ovine, poultry, or fish, and the methods are used, e.g., to enhance non-skeletal muscle tissue function or health in an aging animal. In some such embodiments, the animal is administered a small molecule inhibitor of 15-PGDH. In some embodiments, a vector or expression cassette comprising a nucleic acid inhibitor of 15-PGDH, e.g., an shRNA, is introduced into the animal such that the nucleic acid inhibitor is expressed in the cells of the animal, e.g., the cells of the non-skeletal muscle tissue. In some embodiments, a vector or expression cassette comprising a polynucleotide encoding a polypeptide inhibitor of 15-PGDH, e.g., an antibody or peptide, is introduced into the animal such that the polypeptide inhibitor is expressed in the cells of the animal, e.g., the cells of the non-skeletal muscle tissue. In some embodiments, gene therapy is used, e.g., such that all or part of an endogenous 15-PGDH encoding gene is replaced with a form of the gene that is less active, less stable, or less highly expressed in cells, e.g., non-skeletal muscle tissue cells, of the animal. In some embodiments, modified RNA, e.g., a chemically modified RNA inhibitor such as shRNA or a chemically modified mRNA encoding a polypeptide 15-PGDH inhibitor is introduced into the animal such that the RNA inhibitor or expressed protein inhibitor is present in cells of the animal.

### 6. Assessing 15-PGDH Levels

Any of a number of methods can be used to assess the level of 15-PGDH in a non-skeletal muscle tissue or a skeletal muscle tissue, e.g., when using 15-PGDH as a biomarker or when assessing the efficacy of an inhibitor of 15-PGDH. For example, the level of 15-PGDH can be assessed by examining the transcription of a gene encoding 15-PGDH (e.g., the Hpgd gene), by examining the levels of 15-PGDH protein in the tissue (e.g., skeletal muscle or non-skeletal muscle tissue), or by measuring the 15-PGDH enzyme activity in the tissue (e.g., skeletal muscle or non-skeletal muscle tissue). Such methods can be performed on the overall tissue or on a subset of cells within the tissue, e.g., senescent cells.

In some embodiments, the methods involve the measurement of 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

In some embodiments, the methods involve the detection of 15-PGDH-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of 15-PGDH protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J Chromatogr. B. Biomed. Sci., 699:463-80 (1997)).

### 7. 15-PGDH as a Biomarker

In some embodiments, 15-PGDH may be used as a biomarker for aged skeletal muscle and/or non-skeletal muscle tissue, or for the presence or potential for an age-related condition or disease. For example, a detection of an increase in 15-PGDH levels in skeletal muscle and/or a non-skeletal muscle tissue, e.g., in the overall tissue or in specific cells within the tissue such as senescent cells, is indicative of aging in the tissue, of a loss or decrease of function or health of the tissue related to aging, or of the presence of an age-related condition or disease. For example, a detected increase of 10%, 20%, 30%, 40%, 50%, 60%, 70%, 80%, 90%, 100%, or more 15-PGDH in a skeletal muscle and/or a non-skeletal muscle tissue as compared to in a control tissue from a subject without an age-related condition or disease may be indicative of aging of the tissue, of a loss or decrease of function or health of the tissue related to aging, or of the presence of an age-related condition or disease.

### 8. 15-PGDH Inhibitors

Any agent that reduces, decreases, counteracts, attenuates, inhibits, blocks, downregulates, or eliminates in any way the expression, stability or activity, e.g., enzymatic activity, of 15-PGDH can be used in the present methods. Inhibitors can be small molecule compounds, peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH.

In some embodiments, the 15-PGDH inhibitor decreases the activity, stability, or expression of 15-PGDH by at least 10%, 15%, 20%, 25%, 30%, 35%, 40%, 45%, 50%, 55%, 60%, 65%, 70%, 75%, 80%, 85%, 90%, or more relative to a control level, e.g., in the absence of the inhibitor, in vivo or in vitro.

The efficacy of inhibitors can be assessed, e.g., by measuring 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the 15-PGDH inhibitor is considered effective if the level of expression of a 15-PGDH-encoding polynucleotide is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a 15-PGDH inhibitor is considered effective if the level of expression of a 15-PGDH-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a 15-PGDH inhibitor can also be assessed by detecting protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

For determining whether 15-PGDH protein levels are decreased in the presence of a 15-PGDH inhibitor, the method comprises comparing the level of the protein (e.g., 15-PGDH protein) in the presence of the inhibitor to a reference value, e.g., the level in the absence of the inhibitor. In some embodiments, a 15-PGDH protein is decreased in the presence of an inhibitor if the level of the 15-PGDH protein is decreased by at least 10%, at least 20%, at least 30%, at least 40%, at least 50%, at least 60%, at least 70%, at least 80%, at least 90% or more as compared to the reference value. In some embodiments, a 15-PGDH protein is decreased in the presence of an inhibitor if the level of the 15-PGDH protein is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

### Small Molecules

In particular embodiments, 15-PGDH is inhibited by the administration of a small molecule inhibitor. Any small molecule inhibitor can be used that reduces, e.g., by 10%, 15%, 20%, 25%, 30%, 35%, 40%, 45%, 50%, 55%, 60%, 65%, 70%, 75%, 80%, 85%, 90%, or more, the expression, stability, or activity of 15-PGDH relative to a control, e.g., the expression, stability, or activity in the absence of the inhibitor. In particular embodiments, small molecule inhibitors may be used that can reduce the enzymatic activity of 15-PGDH in vitro or in vivo. Non-limiting examples of small molecule compounds that can be used in the present methods include the small molecules disclosed in publication EP 2838533, the entire disclosure of which is herein incorporated by reference. Small molecules can include, inter alia, the small molecules disclosed in Table 2 of publication EP 2838533, i.e., SW033291, SW033291 isomer B, SW033291 isomer A, SW033292, 413423, 980653, 405320, SW208078, SW208079, SW033290, SW208080, SW208081, SW206976, SW206977, SW206978, SW206979, SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4-(2-cyclohexylethoxy)benzylidene)thiazolidine-2,4-dione, 5-(3-chloro-4-(2-cyclohexylethoxy)benzyl)thiazolidine-2,4-dione, (Z)-N-benzyl-4-((2,4-dioxothiazolidin-5-ylidene)methyl)benzamide, or any of the compounds disclosed in Choi et al. (2013) Bioorganic & Medicinal Chemistry 21:4477-4484; Wu et al. (2010) Bioorg. Med. Chem. 18(2010) 1428-1433; Wu et al. (2011) J Med. Chem. 54:5260-5264; or Yu et al. (2019) Biotechnology and Bioprocess Engineering 24:464-475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins, Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor is 2-((6-bromo-4H-imidazo[4,5-b]pyridin-2-ylthio)methyl)benzonitrile (CID: 3245059), piperidin-1-yl(1-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular Libraries Program [Internet]), the entire disclosure of which is herein incorporated by reference.

In some embodiments, the 15-PGDH inhibitor is TD88 or any of the compounds disclosed in Seo et al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor is EEAH (Ethanol extract of Artocarpus heterophyllus) or any of the compounds disclosed in Kama (2017) Pharmacogn Mag. 2017 January; 13(Suppl 1): S122-S126, the entire disclosure of which is herein incorporated by reference.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a 15-PGDH polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos. NM_000860.6, NM_001145816.2, NM_001256301.1, NM_001256305.1, NM_001256306.1, NM_001256307.1, or NM_001363574.1).

In some embodiments, the methods described herein comprise treating a subject, e.g., a subject with sarcopenia or aging or atrophic muscle; or a subject with an age-related condition, disorder, or disease, using an shRNA or siRNA. A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J Pharmacol. 157:153-165, 2009. In some embodiments, a method of treating a subject, e.g., with aging and/or atrophic muscle; or a subject with an age-related condition, disorder, or disease, comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos. NM_000860.6, NM_001145816.2, NM_001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM 000860.6, NM 001145816.2, NM_001256301.1, NM_001256305.1, NM_001256306.1, NM_001256307.1, or NM_001363574.1). In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., 95%, 90%, 85%, 80%, 75%, or 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies, 4^{th} edition by N.S. Templeton; Translating Gene Therapy to the Clinic: Techniques and Approaches, 1^{st} edition by J. Laurence and M. Franklin; High-Throughput RNAi Screening: Methods and Protocols (Methods in Molecular Biology) by D.O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see, e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function, e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet™ (ForteBio, Inc., Menlo Park, Calif.)), and western blot analysis.

### Peptides

In some embodiments, the agent is a peptide, e.g. a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and Modified RNA

In some embodiments, polynucleotides providing 15-PGDH inhibiting activity, e.g., a nucleic acid inhibitor such as an siRNA or shRNA, or a polynucleotide encoding a polypeptide that inhibits 15-PGDH, are introduced into cells, e.g., muscle cells, non-skeletal muscle tissue cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitors, e.g., a nucleic acid inhibitor or a polynucleotide encoding a polypeptide inhibitor, are introduced into cells, e.g., muscle cells, non-skeletal muscle tissue cells, using vectors such as viral vectors. Suitable viral vectors include but not limited to adeno-associated viruses (AAVs), adenoviruses, and lentiviruses. In some embodiments, a 15-PGDH inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the skeletal muscle or non-skeletal muscle tissues of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used. See, e.g., Youn et al. (2015) Expert Opin Biol Ther, Sep. 2; 15(9): 1337-1348; Kaczmarek et al. (2017) Genome Medicine 9:60,; Gan et al. (2019) Nature comm. 10: 871; Chien et al. (2015) Cold Spring Harb Perspect Med. 2015;5:a014035; the entire disclosures of each of which are herein incorporated by reference.

### 9. Methods of Administration

The compounds described herein can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

Administration of the compound into a subject can be accomplished by methods generally used in the art. The quantity of the compound introduced may take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the condition or disease, including reducing or eliminating one or more symptoms or manifestations of the condition or disease.

Any number of muscles of the body may be directly injected with or otherwise administered the compounds described herein, such as, for example, the biceps muscle; the triceps muscle; the brachioradialus muscle; the brachialis muscle (brachialis anticus); the superficial compartment wrist flexors; the deltoid muscle; the biceps femoris, the gracilis, the semitendinosus and the semimembranosus muscles of the hamstrings; the rectus femoris, vastus lateralis, vastus medialis and vastus intermedius muscles of the quadriceps; the gastrocnemius (lateral and medial), tibialis anterior, and the soleus muscles of the calves; the pectoralis major and the pectoralis minor muscles of the chest; the latissimus dorsi muscle of the upper back; the rhomboids (major and minor); the trapezius muscles that span the neck, shoulders and back; the rectus abdominis muscles of the abdomen; the gluteus maximus, gluteus medius and gluteus minimus muscles of the buttocks; muscles of the hand; sphincter muscles; ocular muscles; and pharyngeal muscles.

The compounds described herein may be administered locally by injection into the non-skeletal muscle tissue being targeted, or by administration in proximity to the tissue being targeted.

### 10. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see, e.g., REMINGTON'S PHARMACEUTICAL SCIENCES, 18TH ED., Mack Publishing Co., Easton, Pa. (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (e.g., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side effects caused by induction of the immune response, etc. In some embodiments, for example, the 15-PGDH inhibitors described herein are administered together with a senolytic agent, a compound to enhance PGE2 levels or PGD2 levels, a compound to decrease Atrogin1 levels or activity, a compound to increase signaling through the EP1, EP2, EP3, EP4, DP1, and/or DP2 receptors, and/or any other compound aiming to enhance muscle mass, strength, or function; or the function, health, or any other desired property of the non-skeletal muscle tissue being targeted.

### 11. Kits

Other embodiments of the compositions described herein are kits comprising a 15-PGDH inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of aging and/or atrophied muscle. In some embodiments, the kit comprises one or more reagents for the treatment of a non-skeletal muscle tissue in a subject with an age-related condition, disorder, or disease. In some embodiments, the kit comprises an agent that antagonizes the expression or activity of 15-PGDH. In some embodiments, the kit comprises an inhibitory nucleic acid (e.g., an antisense RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises an antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor, e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH.

In some embodiments, the kits can further comprise instructional materials containing directions (e.g., protocols) for the practice of the methods described herein (e.g., instructions for using the kit for enhancing mass, strength, or function in aged and/or atrophied muscle; and/or for using the kit for enhancing the function, health, or other properties of non-skeletal muscle tissues). While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Targeting Prostaglandin E2 Degrading Enzyme Ameliorates Sarcopenia and Muscular Dystrophy

### Abstract

Sarcopenia is a muscle wasting syndrome associated with aging that to date lacks effective therapeutic approaches. Here, we have identified that a loss of PGE2 levels contributes to muscle atrophy in aged skeletal muscle. We reveal that accumulation of senescent cells in aged muscle contributes to elevated PGE2 degrading enzyme (15-PGDH) levels. Using a pharmacological agent, SW033291, to inhibit the 15-PGDH enzyme or gene therapy to knockdown 15-PGDH, we have observed increases in muscle mass, strength and exercise performance of aged mice. We have observed similar reductions in 15-PGDH and increases in strength in mice with Duchenne muscular dystrophy (mdxcv4/mTRKO(G2)). Using a systemic senolytic treatment (ABT-263), we have shown that 15-PGDH levels are reduced in muscle tissues. Using genetic and cell culture models, we have uncovered the role of Prostaglandin E2 (PGE2) signaling through the EP4 receptor in differentiated cells and myofibers as a regulator of muscle mass. PGED signaling inhibits Atrogin1 expression, a crucial mediator of muscle atrophy. Here we have uncovered 15-PGDH inhibition, the prostaglandin E2 degrading enzyme, as an effective target to reverse muscle mass and strength loss and counter aging and muscular dystrophy.

### INTRODUCTION

Atrophy results from a rapid loss of muscle mass and strength primarily due to excessive protein breakdown, which frequently is accompanied by diminished protein synthesis. Quality of life is reduced and morbidity and mortality are increased due to this loss of muscle function. While much is known about how muscle atrophy arises, current therapeutic strategies to effectively prevent or slow atrophy are limited to exercise. Experimental approaches currently under investigation are largely directed at increasing muscle mass by altering protein balance, e.g., via myostatin inhibitors (1).

Here we tested if modulation of the PGE2 pathway could increase function in atrophied muscles of aged mice. We made the unexpected finding that PGE2 catabolism is dysregulated, leading to detrimental effects on aged murine muscle tissues. We reveal that in aged muscle tissues, PGE2 is detected at lower levels than in young, a finding not previously associated with aged muscles. We uncover the cellular and molecular basis for the dysregulation of PGE2 synthesis, catabolism and signaling in aged muscles. We design a strategy to increase PGE2 levels by inhibition of 15-PGDH, the catabolic enzyme that renders PGE2 inactive, detectable as PGE2 metabolites (PGEM) in aged muscle tissue. 15-PGDH inhibition surmounts deleterious effects of the aged muscle microenvironment, leading to a robust increase in strength, muscle mass and endurance in aged mice.

### Discovery of Decreased PGE2 Levels in Aged Muscle Tissues

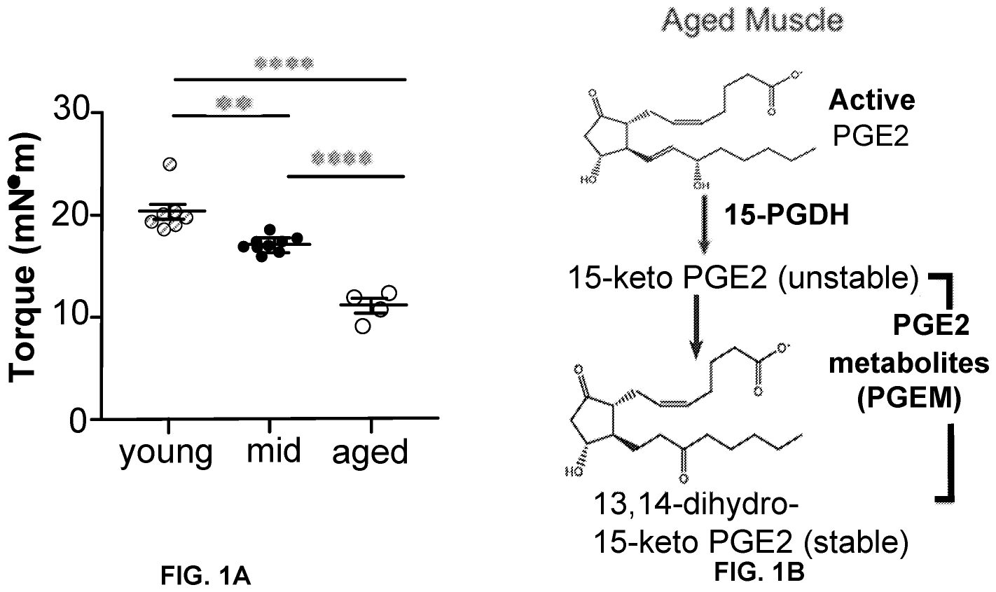

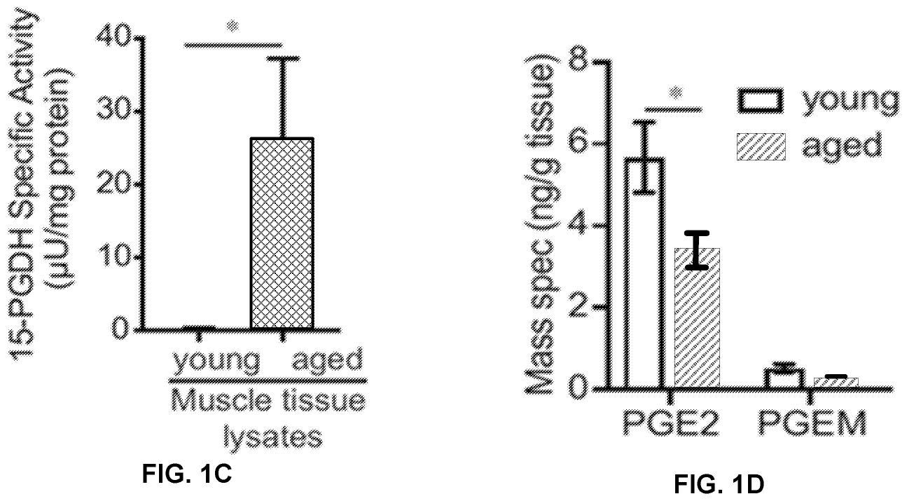

A progressive decline in muscle strength accompanies aging, as shown here for the Gastrocnemius (GA) muscles of mice assessed at different ages by plantar flexion torque (FIG. 1A). PGE2 is catabolized by a 2-step process wherein the first is mediated by the rate limiting enzyme 15-hydroxyprostaglandin dehydrogenase (15-PGDH) and involves conversion of PGE2 to the labile 15-keto-PGE2, and the second step is mediated by prostaglandin reductase 2 and involves conversion of 15-keto-PGE2 to the more stable 13,14-dihydro-15-keto-PGE2 metabolite (3, 4) (FIG. 1B). In accordance with a decrease in PGE2, 15-PGDH activity was dramatically increased in geriatric muscle tissue (FIG. 1C). Further analysis of the PGE2 signaling pathway during aging uncovered that levels of PGE2 were lower in aged muscles as shown by mass spectrometry analysis (FIG. 1D). Together this suggests that PGE2 is catabolized in the aged muscle microenvironment, or niche.

### Catabolism of PGE2 is Via 15-PGDH Upregulation in Senescent Cells in Aged Tissues

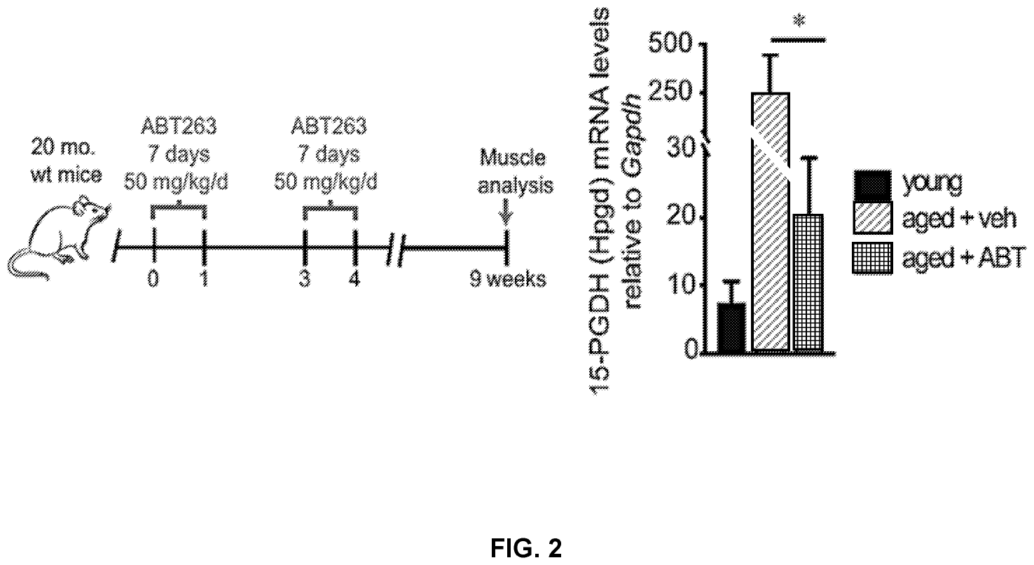

Senescent cells have been reported to accumulate and adversely affect tissue function with aging. PGE2 has been postulated to be a component of the senescence associated secretory phenotype (SASP) (5, 6). We hypothesized that 15-PGDH expression and PGE2 inactivation was due to senescent cells in aged muscle. To address this possibility, we treated aged mice (20 months) with a senolytic agent, ABT-263, also known as navitoclax, which acts by inhibiting Bc1-2, Bc1-w and Bc1-xL, to induce apoptosis in senescent cells (7) (FIG. 2A). After two months of ABT-263 treatment, the levels of the PGE2 degrading enzyme (15-PGDH) mRNA were markedly decreased (FIG. 2A), indicating that a major cell source in aged muscles is senescent cells that are eliminated from the tissue by senolytic treatment. These results suggest that PGE2 inactivation is mediated in part by senescent cells in the aged muscle tissue that contribute to the muscle wasting phenotype associated with aging.

### 15-PGDH Inhibition Leads to Improvement of Muscle Function in Aged Mice

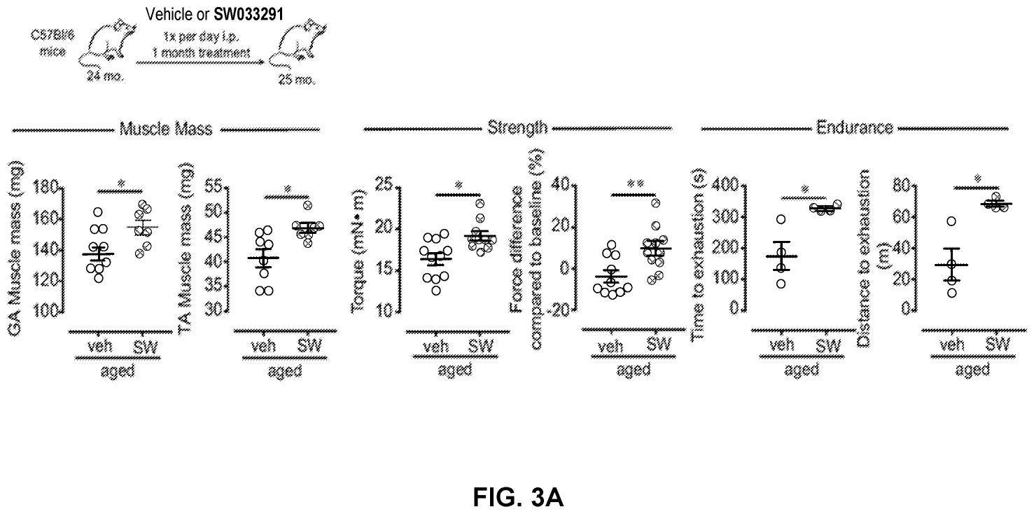

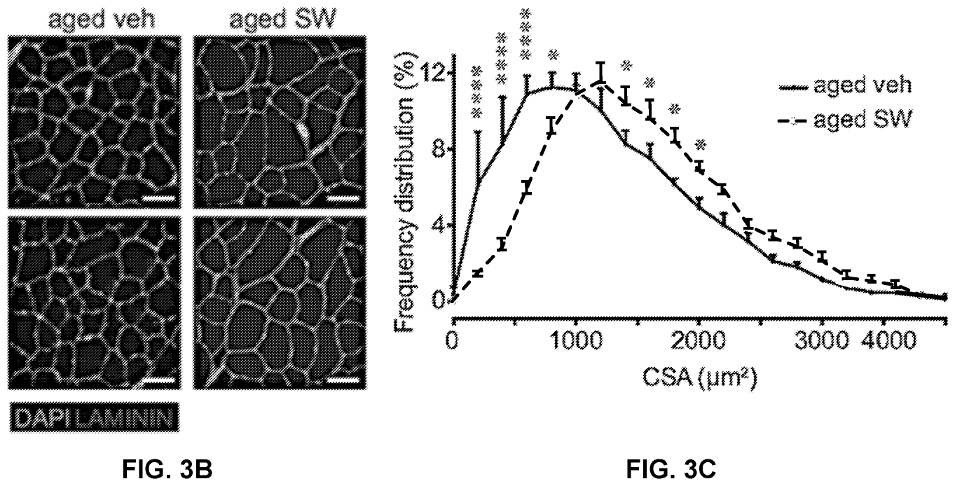

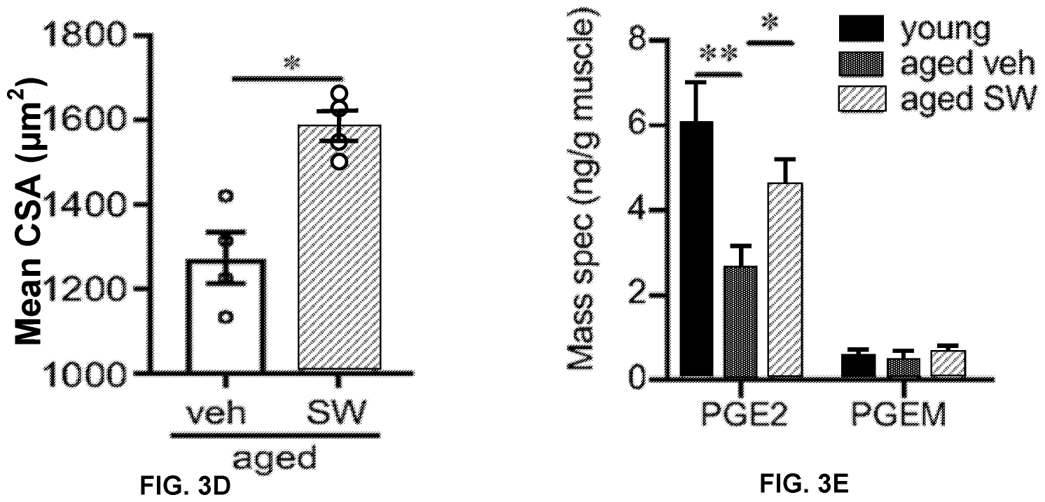

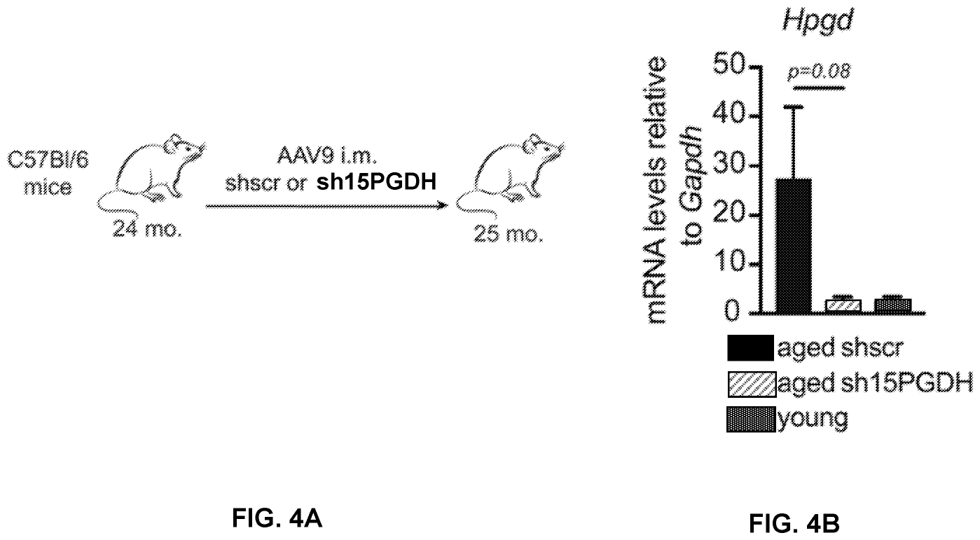

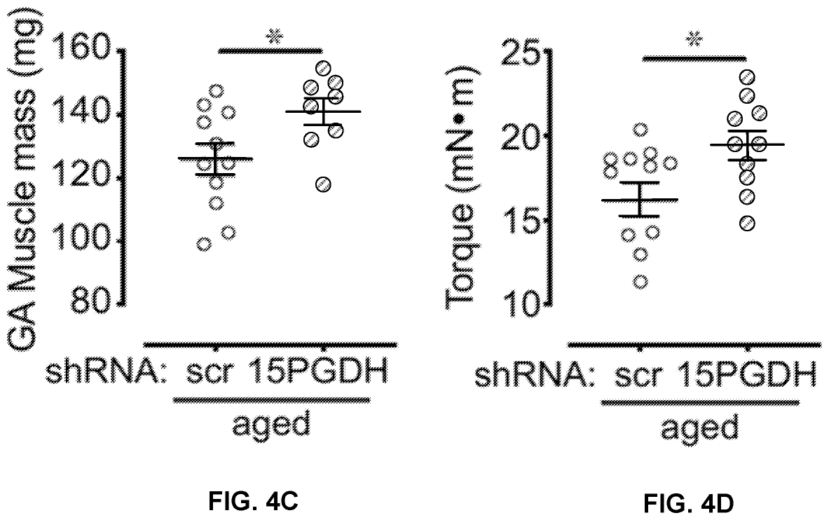

We sought to determine if PGE2 inactivation was a major component of muscle wasting and the decrease in muscle function in aged mice. We treated aged mice with a 15-PGDH inhibitor, SW033291 (SW), daily for 1 month and found that 15-PGDH inhibition led to a significant increase in muscle mass, strength and endurance in aged mice (FIG. 3A). We performed histological analysis and found that the myofiber cross-sectional area was larger in SW-treated aged mice (FIG. 3B-D). To confirm that the phenotype was due to increased levels of PGE2, we performed mass spectrometry on the muscle samples, and found SW treatment elevated the PGE2 levels in muscles comparable to levels in young muscles (FIG. 3E). To ascertain the effect was through inhibition of 15-PGDH, we used an independent method through knockdown of the enzyme by use of an shRNA (sh15PGDH) delivered to aged muscles by an adeno-associated virus AAV9 (FIG. 4A). We confirmed levels of Hpgd (15-PGDH) in the AAV9 mediated sh15PGDH knockdown were reduced at the mRNA level by qPCR as compared to the AAV9 mediated shRNA scramble (shscr) control (FIG. 4B). We found muscle mass and muscle force were increased compared to muscles of controls infected with AAV (shscr) (FIG. 4C,D).

### 15-PGDH Inhibition Leads to Improvement of Muscle Function in Duchenne Mice

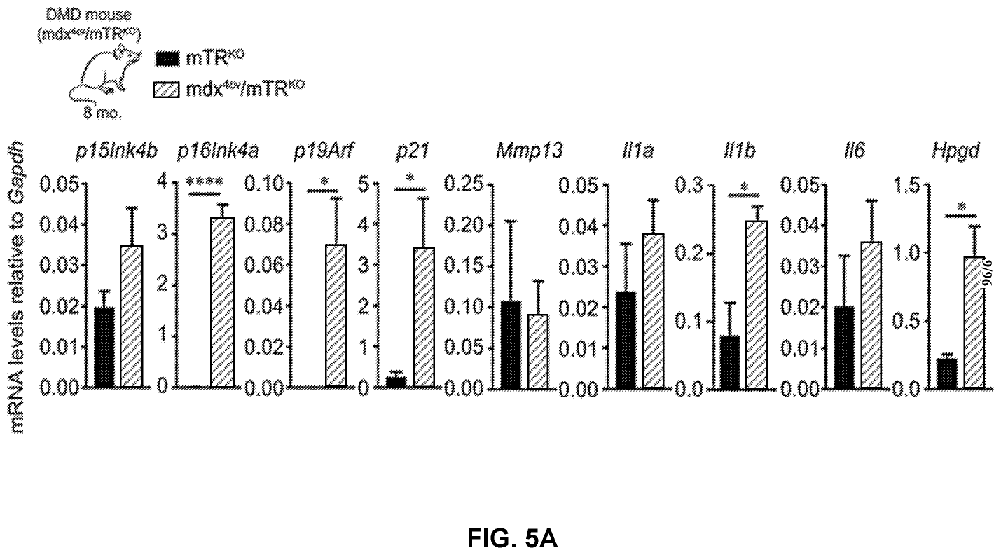

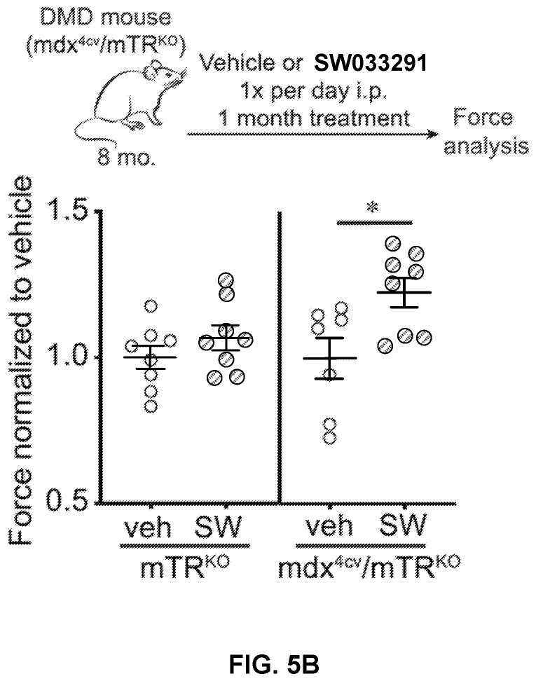

To extend our finding to other muscle wasting diseases characterized by muscle atrophy and high senescent cell infiltration, we analyzed the mdx4cv/mTRKO(G2) Duchenne muscular dystrophy (DMD) mouse model with “humanized” telomere lengths, which recapitulates the skeletal muscle and heart DMD phenotype (8,9). By qPCR we analyzed the levels of senescent and senescence-associated secretory phenotype (SASP) markers and found them to be greatly elevated in the mdx4cv/mTRKO(G2) mice (10) (FIG. 5A). Importantly, we found the degrading enzyme, 15-PGDH, to be significantly increased in the mdx4cv/mTRKO(G2) as compared to the mTRKO(G2) controls (FIG. 5A). To elucidate if PGE2 inactivation contributed to the muscle wasting seen in DMD, we treated 8 month old mdx4cv/mTRKO(G2) and mTRKO(G2) controls with SW and observed an increase of 22% in muscle strength in these mice compared to vehicle treated controls after 4 weeks of treatment (FIG. 5B).

### PGE2 Prevents Atrophy Through the EP4 Receptor in Muscle Fibers

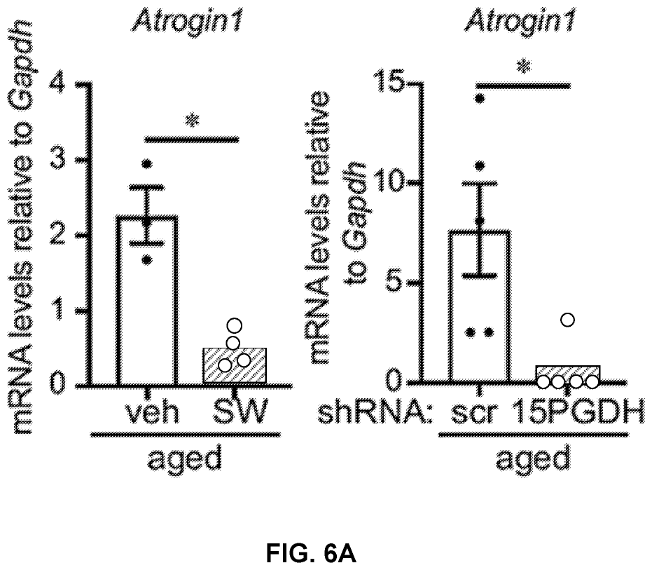

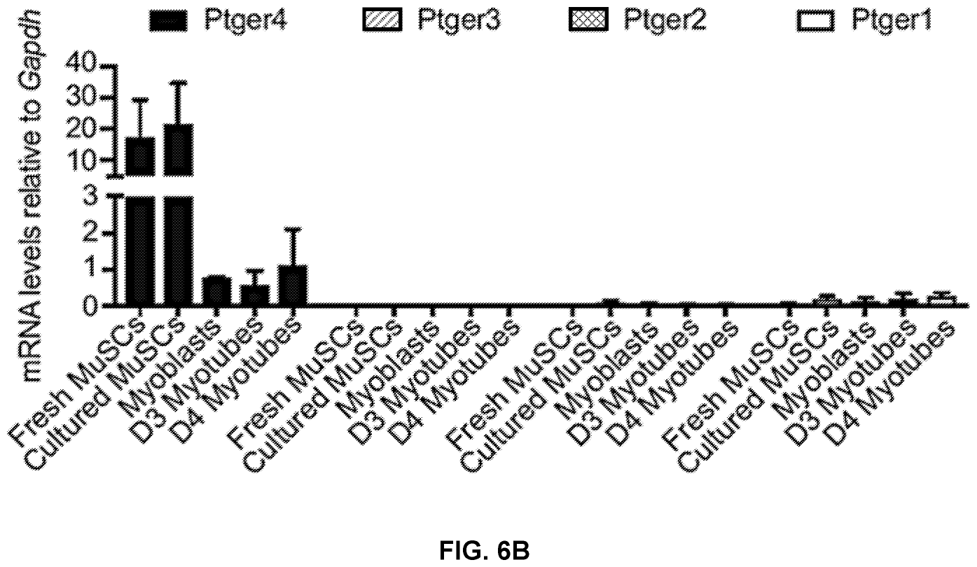

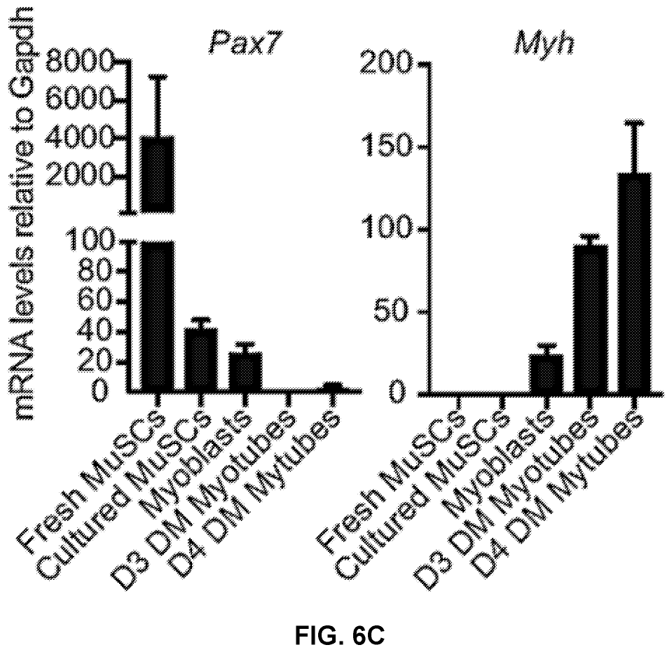

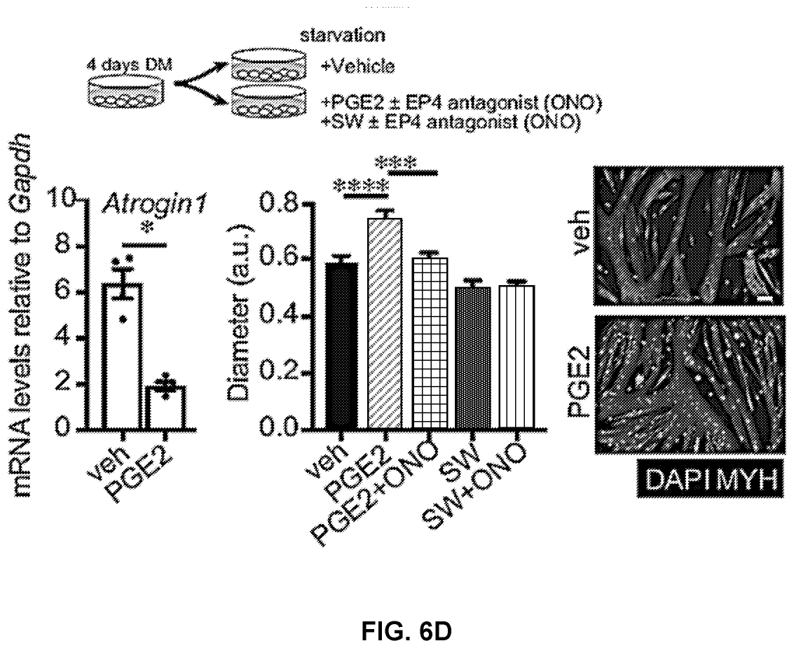

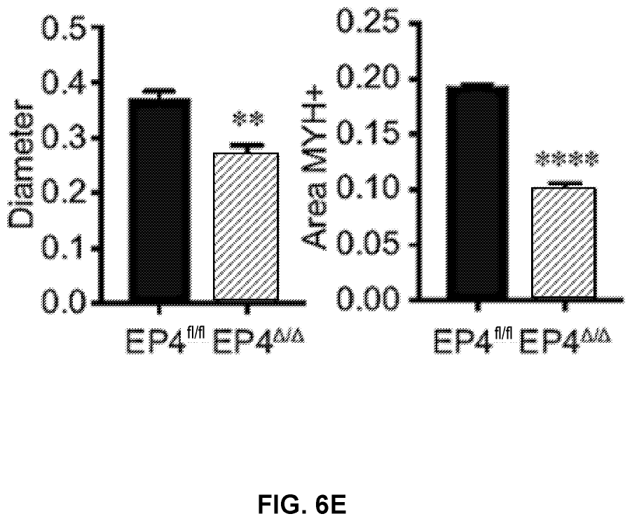

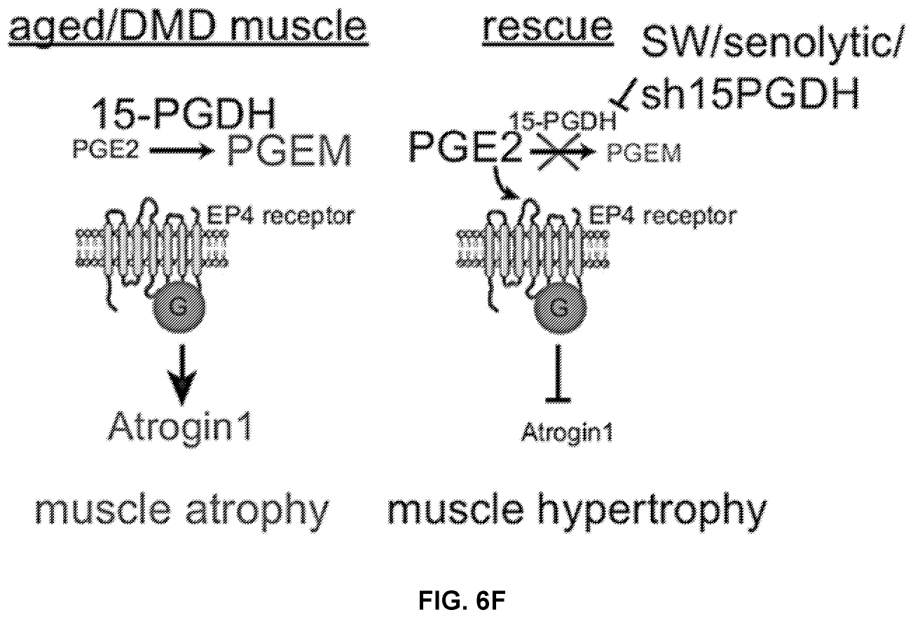

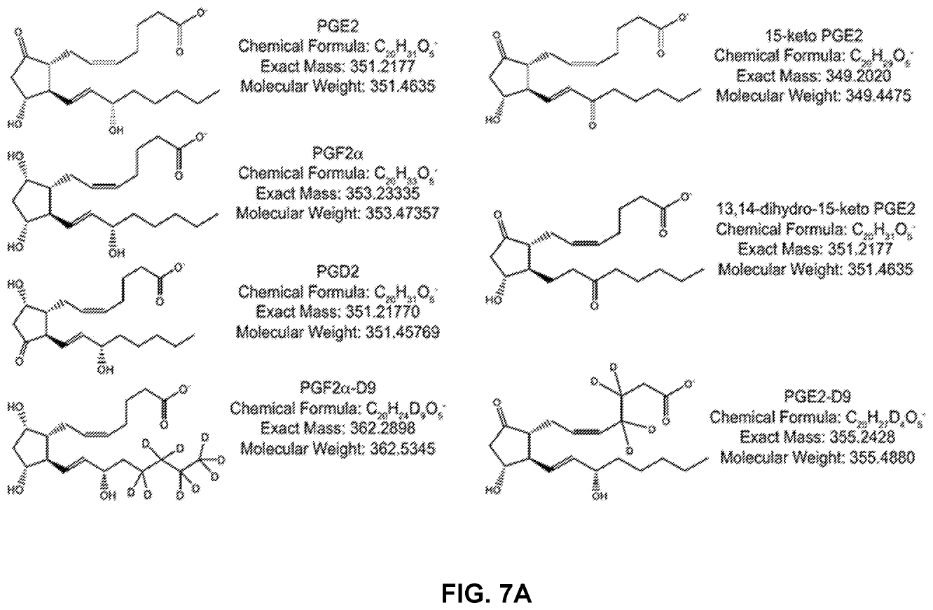

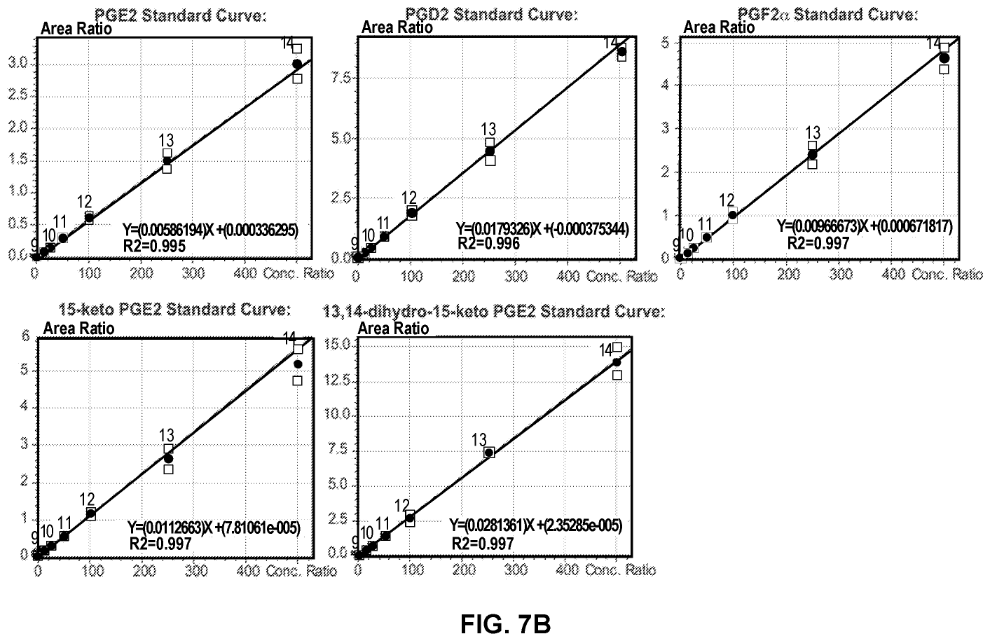

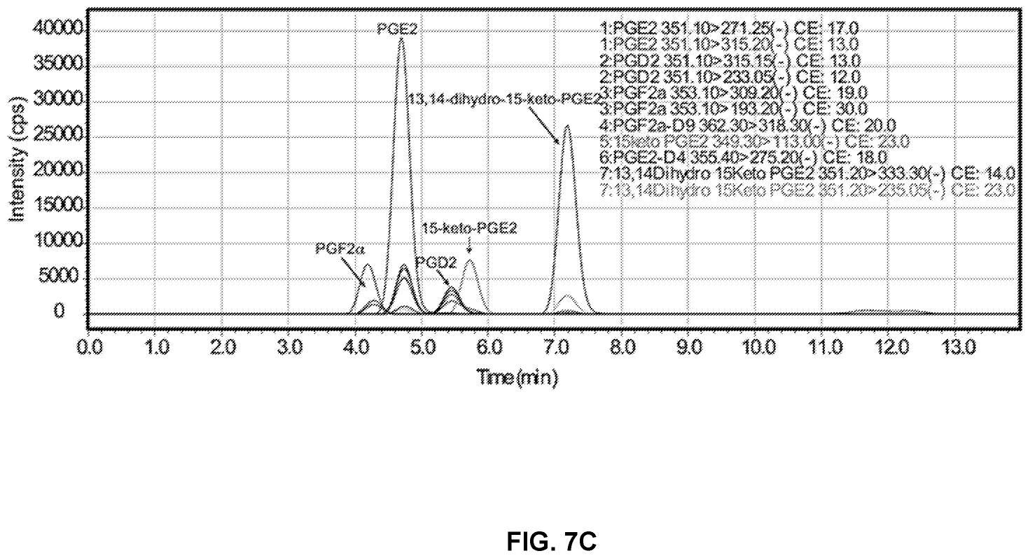

To understand the downstream mechanism by which 15-PGDH inhibition leads to amelioration of muscle atrophy, we performed qPCR analysis of aged muscles treated with SW or AAV-sh15PGDH. We hypothesized that PGE2 stimulation of the EP4 receptor could be responsible for the amelioration of the atrophy phenotype through inhibition of ATROGINI (11-14). Our data confirm that SW treatment and knockdown of 15-PGDH by AAV9 sh15PGDH delivery leads to decreased expression of Fbxo32 (Atrogin1) at the mRNA level (FIG. 6A).

To further delineate the mechanism of action of PGE2 in the muscle, we tested if PGE2 signals through the EP4 receptor in differentiated myotubes. We analyzed the levels of all of the PGE2 receptors, EP1-EP4 (Ptger1-4), and as previously described, we found EP4 to be highly expressed in muscle stem cells (MuSCs) (15). However, we also found that EP4 is expressed in differentiated myoblasts and myotubes, albeit at lower levels than in MuSCs (FIG. 6B,C). To mimic atrophy in vitro and elucidate the effects of PGE2 signaling, we treated starved myotubes with either vehicle, PGE2 or PGE2 in the presence of the EP4 antagonist (ONO-AE3-208). We found that PGE2 greatly decreased Atrogin1 expression in starved myotubes (FIG. 6D). Additionally, we found that PGE2 increased myotube diameter in starved or non-starved cultured myotubes (FIG. 6D). In the presence of the EP4 antagonist (ONO-AE3-208), this effect was abrogated, providing evidence that PGE2 promotes hypertrophy in myotubes through the EP4 receptor (FIG. 6D). To ascertain if SW could mediate effects on myotubes independent of PGE2, we assessed its effects on cultured myotubes. In the absence of senescent cells or other cells expressing 15-PGDH, we found SW treated starved myofibers exhibited no increase in myotube diameter (FIG. 6D), in contrast to the increase in myofiber cross-sectional area observed following SW treatment in vivo (FIG. 3B-D). To confirm the role of the EP4 receptor in myotubes, we used EP4flox/flox myoblasts in which the receptor is genetically ablated following infection with a cre-expressing lentivirus with empty vector serving as a control. In the absence of EP4 receptors, smaller myotubes were observed, suggesting that the EP4 receptor plays a key role in myotube differentiation (FIG. 6E). These results reveal a role for PGE2 signaling through the EP4 receptor in muscle atrophy. Further, we demonstrate that, like muscle tissue, PGE treatment of cultured myotubes inhibits an atrophy-related ubiquitin ligase, Atrogin1 (FIG. 6F).

### Discussion

We uncover 15-PGDH as a therapeutic target in aging and dystrophic muscle that, when reduced, ameliorates muscle atrophy. We previously showed the importance of PGE2 signaling in muscle stem cell (MuSC) function in the context of young muscle regeneration (15). This entailed transplantation of PGE2-treated MuSCs into a damaged muscle or localized intramuscular delivery of PGE2 to the damaged muscle. Prior work has implicated 15-PGDH inhibition in regeneration in young mice and shown that systemic delivery of the small molecule inhibitor of 15-PGDH, SW033291, is a potent inducer of endogenous PGE2 that improves hematopoietic, liver and colon tissue regeneration (16). Here we show that 15-PGDH has a previously unrecognized role in muscle aging. Expressed only at low levels in young muscle tissue, 15-PGDH levels increase as senescent cells accumulate. Further, we show that inhibition of 15-PGDH ameliorates skeletal muscle function in aged mice. The systemic reconstitution of endogenous PGE2 levels by preventing its degradation in muscle ameliorates muscle atrophy, leading to increased mass and strength. Our findings provide unexpected evidence for a role of the PGE2 degrading enzyme in muscle wasting diseases such as DMD and aging and show that it constitutes a potent therapeutic target.

### REFERENCES

1. S. Cohen, J. A. Nathan, A. L. Goldberg, Muscle wasting in disease: molecular mechanisms and promising therapies. Nat Rev Drug Discov 14, 58-74 (2015).
2. B. Pawlikowski, C. Pulliam, N. D. Betta, G. Kardon, B. B. Olwin, Pervasive satellite cell contribution to uninjured adult muscle fibers. Skelet Muscle 5, 42 (2015).
3. D. Wang, R. N. Dubois, Eicosanoids and cancer. Nat Rev Cancer 10, 181-193 (2010).
4. Y. H. Wu et al., Structural basis for catalytic and inhibitory mechanisms of human prostaglandin reductase PTGR2. Structure 16, 1714-1723 (2008).
5. J.-P. Coppe, P.-Y. Desprez, A. Krtolica, J. Campisi, The senescence-associated secretory phenotype: the dark side of tumor suppression. Annual review of pathology 5, 99-118 (2010).
6. N. N. Huang, D. J. Wang, L. A. Heppel, Stimulation of aged human lung fibroblasts by extracellular ATP via suppression of arachidonate metabolism. Journal of Biological Chemistry 268, 10789-10795 (1993).
7. J. Chang et al., Clearance of senescent cells by ABT263 rejuvenates aged hematopoietic stem cells in mice. Nature Medicine 22, 78-83 (2016).
8. F. Mourkioti et al., Role of telomere dysfunction in cardiac failure in Duchenne muscular dystrophy. Nat Cell Biol 15, 895-904 (2013).
9. A. Sacco et al., Short telomeres and stem cell exhaustion model Duchenne muscular dystrophy in mdx/mTR mice. Cell 143, 1059-1071 (2010).
10. I. Le Roux, J. Konge, L. Le Cam, P. Flamant, S. Tajbakhsh, Numb is required to prevent p53-dependent senescence following skeletal muscle injury. Nat Commun 6, 8528 (2015).
11. H. Fujino, J. W. Regan, EP(4) prostanoid receptor coupling to a pertussis toxin-sensitive inhibitory G protein. Mol Pharmacol 69, 5-10 (2006).
12. V. Konya, G. Marsche, R. Schuligoi, A. Heinemann, E-type prostanoid receptor 4 (EP4) in disease and therapy. Pharmacol Ther 138, 485-502 (2013).
13. M. Sandri et al., Foxo transcription factors induce the atrophy-related ubiquitin ligase atrogin-1 and cause skeletal muscle atrophy. Cell 117, 399-412 (2004).
14. T. N. Stitt et al., The IGF-1/PI3K/Akt pathway prevents expression of muscle atrophy-induced ubiquitin ligases by inhibiting FOXO transcription factors. Mol Cell 14, 395-403 (2004).
15. A. T. V. Ho et al., Prostaglandin E2 is essential for efficacious skeletal muscle stem-cell function, augmenting regeneration and strength. Proc Natl Acad Sci USA 114, 6675-6684 (2017).
16. Y. Zhang et al., TISSUE REGENERATION. Inhibition of the prostaglandin-degrading enzyme 15-PGDH potentiates tissue regeneration. Science 348, aaa2340 (2015).

Materials and Methods

### Mice

We performed all experiments and protocols in compliance with the institutional guidelines of Stanford University and Administrative Panel on Laboratory Animal Care (APLAC). Mid (18 mo.) and aged (>24 mo.) mice C57BL/6 were obtained the US National Institute on Aging (NIA) for aged muscle studies, and young (2-4 mo.) wild-type C57BL/6 mice from Jackson Laboratory. Mice were maintained in specific-pathogen free housing on a 12-hour dark/light cycle for study duration.

For ABT-263 treatments, 20 month old C57/B16 mice were treated with vehicle (ethanol:polyethylene glycol 400:Phosal 50 PG) or ABT-263 (in ethanol:polyethylene glycol 400:Phosal 50 PG) by oral gavage for 2 cycles of 1 week with a 2 week rest period between cycles as described previously (1). For the Duchenne muscular dystrophy (DMD) mouse model, we used 8-10 month old mdx4cv/mTRKO(G2) generated as previously described (2).

Mice were treated for 1 month with SW033291 (SW) (Cayman Chemicals) or vehicle as previously described (3). Time and distance to exhaustion was performed as previously described (4) for SW-treated mice and their controls (FIG. 3A).

Mouse transgenic strains were purchased from The Jackson Laboratory (EP4flox/flox) No. 028102. We validated these genotypes by appropriate PCR-based strategies. Studies were performed with female and male mice unless specified.

### Immunofluorescence Staining and Imaging

We collected and prepared recipient Tibialis anterior (TA) or gastrocnemius (GA) muscle tissues for histology as previously described (5). We fixed transverse sections from muscles using 4% PFA, blocked and permeabilized using PBS/1% BSA/0.1% Triton X-100 and incubated with anti-LAMININ (Millipore, clone A5, catalog #05-206, 1:200) and then with AlexaFluor secondary Antibodies (Jackson ImmunoResearch Laboratories, 1:200) or wheat germ agglutinin-Alexa 647 conjugate (WGA, Thermo Fisher Scientific). We counterstained nuclei with DAPI (Invitrogen).

For myotubes we performed fixation using 4% PFA, blocking and permeabilization using PBS/1% BSA/0.1% Triton X-100 and staining with primary antibodies anti-MyHC (Thermo Fisher Scientific, catalog #14-6503-82, clone MF-20, 1:500) and then with AlexaFluor secondary Antibodies (Jackson ImmunoResearch Laboratories, 1:500). We counterstained nuclei with DAPI (Invitrogen).

We acquired images on a Zeiss 510 laser scanning confocal microscope (Carl Zeiss Microimaging) with 40x/0.9N.A. objective to capture multiple consecutive focal planes or using the KEYENCE BZ-X700 all-in-one fluorescence microscope (Keyence) with 20x/0.75 N.A. objectives. We analyzed the myofiber area using the Keyence Advanced Analysis Software. For cross sectional area the maximum cross sectional area of the muscle was quantified or at least 10 fields of LAMININ-stained myofiber cross-sections encompassing over 400 myofibers were captured for each mouse as above. Data analyses were blinded. The researchers performing the imaging acquisition and scoring were unaware of treatment condition given to sample groups analyzed.

### Cell Culture

Primary myoblasts were grown in myogenic cell culture medium containing DMEM/F10 (50:50), 15% FBS, 2.5 ng ml-1 fibroblast growth factor-2 and 1% penicillin-streptomycin. For differentiation experiments, confluent myoblasts were grown in medium containing 5% horse serum, DMEM. We added 10 ng/ml Prostaglandin E2 (Cayman Chemicals), 1 μM of SW033291 (ApexBio) or 1 μM of ONO-AE3-208 (Cayman Chemicals) to day 4 differentiated myotubes. Myoblasts were isolated from EP4^{fl/fl} mice and received either a mCherry/Cre lentivirus or a mock infection as previously described (5).

### Quantitative RT-PCR

We isolated RNA from MuSCs using the RNeasy Micro Kit (Qiagen). For muscle samples, we snap froze the tissue in liquid nitrogen, homogenized muscles in Trizol (Invitrogen) using the FastPrep FP120 homogenizer (MP Biomedicals), and then isolated RNA. We reverse-transcribed cDNA from total mRNA from each sample using the SensiFAST™ cDNA Synthesis Kit (Bioline). We subjected cDNA to RT-PCR using SYBR Green PCR Master Mix (Applied Biosystems) or TaqMan Assays (Applied Biosystems) in an ABI 7900HT Real-Time PCR System (Applied Biosystems). We cycled samples at 95° C. for 10 min and then 40 cycles at 95° C. for 15 s and 60° C. for 1 min. To quantify relative transcript levels, we used 2-ΔΔCt to compare treated and untreated samples and expressed the results relative to Gapdh.

For SYBR Green qRT-PCR, we used the following primer sequences: Gapdh, 30 forward 5′-TTCACCACCATGGAGAAGGC-3′, reverse 5′-CCCTTTTGGCTCCACCCT-3′; Hpgd, forward 5′-TCCAGTGTGATGTGGCTGAC -3′, reverse 5′-ATTGTTCACGCCTGCATTGT-3′; Ptger1, forward 5′ GTGGTGTCGTGCATCTGCT-3′, reverse 5′-CCGCTGCAGGGAGTTAGAGT-3′, Ptger2, forward 5′-ACCTTCGCCATATGCTCCTT-3′, reverse 5′-GGACCGGTGGCCTAAGTATG-3′, Cox2, forward, 5′-AACCCAGGGGATCGAGTGT-3′, reverse 5′-CGCAGCTCAGTGTTTGGGAT-3′; Fbxo32, forward 5′-TAGTAAGGCTGTTGGAGCTGATAG-3′, reverse 5′-CTGCACCAGTGTGCATAAGG-3′. For murine senescence markers and senescence associated markers we used the previously described primers (6).

TaqMan Assays (Applied Biosystems) were used to quantify Pax7, Myh, p21, Ptger3 and Ptger4 in samples according to the manufacturer instructions with the TaqMan Universal PCR Master Mix reagent kit (Applied Biosystems). Transcript levels were expressed relative to Gapdh levels. For SYBR Green qPCR, Gapdh qPCR was used to normalize input cDNA samples. For Taqman qPCR, multiplex qPCR enabled target signals (FAM) to be normalized individually by their internal Gapdh signals (VIC).

### 15-PGDH Kinetic Assay

### 15-PGDH Activity was Analyzed in Muscle Lysates Using the BioVision PicoProbe 15-PGDH Activity Assay Kit (Cat # K562) according to the protocol of the manufacturer

### Mass Spectrometry

### Analytes:

All prostaglandin standards-PGF2a; PGE2; PGD2; 15-keto PGE2; 13,14-dihydro 15-keto PGE2; PGE2-D4; and PGF2α-D9—were purchased from Cayman Chemical. For the PGE2-D4 internal standard, positions 3 and 4 were labeled with a total of four deuterium atoms. For PGF2α-D9, positions 17, 18, 19 and 20 were labeled with a total of nine deuterium atoms.

### Calibration Curve Preparation:

Analyte stock solutions (5 mg/mL) were prepared in DMSO. These stock solutions were serially diluted with acetonitrile/water (1:1 v/v) to obtain a series of standard working solutions, which were used to generate the calibration curve. Calibration curves were prepared by spiking 10 uL of each standard working solution into 200 μL of homogenization buffer (acetone/water 1:1 v/v; 0.005% BHT to prevent oxidation) followed by addition of 10 uL internal standard solution (3000 ng/mL each PGF2α-D9 and PGE2-D4). A calibration curve was prepared fresh with each set of samples. Calibration curve ranges: for PGE2 and 13,14-dihydro 15-keto PGE2, from 0.05 ng/mL to 500 ng/mL; for PGD2 and PGF2u, from 0.1 ng/mL to 500 ng/mL; and for 15-keto PGE2, from 0.025 ng/mL to 500 ng/mL.

### Extraction Procedure:

The extraction procedure was modified from that of Prasain et al. (7) and included acetone protein precipitation followed by 2-step liquid-liquid extraction; the latter step enhances LC-MS/MS sensitivity. Butylated hydroxytoluene (BHT) and evaporation under nitrogen (N2) gas were used to prevent oxidation.

Solid tissues were harvested, weighed, and snap-frozen with liquid nitrogen. Muscle tissue was combined with homogenization beads and 200 μL homogenization buffer in a polypropylene tube and processed in a FastPrep 24 homogenizer (MP Biomedicals) for 40 seconds at a speed of 6 m/s. After homogenization, 10 μL internal standard solution (3000 ng/mL) was added to tissue homogenate followed by sonication and shaking for 10 minutes. Samples were centrifuged and the supernatant was transferred to a clean Eppendorf tube. 200 μL hexane was added to the sample, followed by shaking for 15 minutes, then centrifugation. Samples were frozen at −80° C. for 40 minutes. The hexane layer was poured off from the frozen lower aqueous layer, and discarded. After thawing, 25 μL of 1N formic acid was added to the bottom aqueous layer, and the samples were vortexed. For the second extraction, 200 μL chloroform was added to the aqueous phase. Samples were shaken for 15 minutes to ensure full extraction. Centrifugation was performed to separate the layers. The lower chloroform layer was transferred to a new Eppendorf tube and evaporated to dryness under nitrogen at 40° C. The dry residue was reconstituted in 100 μL acetonitrile/10 mM ammonium acetate (2:8 v/v) and analyzed by LC-MS/MS.

### LC-MS/MS:

Since many prostaglandins are positional isomers with identical masses and have similar fragmentation patterns, chromatographic separation is critical. Two SRM transitions-one quantifier and one qualifier-were carefully selected for each analyte. Distinctive qualifier ion intensity ratios and retention times were essential to authenticate the target analytes. All analyses were carried out by negative electrospray LC-MS/MS using an LC-20ADXR prominence liquid chromatograph and 8030 triple quadrupole mass spectrometer (Shimadzu). HPLC conditions: Acquity UPLC BEH C18 2.1×100 mm, 1.7 um particle size column was operated at 50° C. with a flow rate of 0.25 mL/min. Mobile phases consisted of A: 0.1% acetic acid in water and B: 0.1% acetic acid in acetonitrile. Elution profile: initial hold at 35% B for 5 minutes, followed by a gradient of 35%-40% in 3 minutes, then 40%-95% in 3 minutes; total run time was 14 minutes. Injection volume was 20 uL. Using these HPLC conditions, we achieved baseline separation of the analytes of interest.

Selected reaction monitoring (SRM) was used for quantification. The mass transitions were as follows: PGD2: m/z 351.10→m/z 315.15 (quantifier) and m/z 351.10-m/z 233.05 (qualifier); PGE2: m/z 351.10→m/z 271.25 (quantifier) and m/z 351.10→m/z 315.20 (qualifier); PGF2a: m/z 353.10→m/z 309.20 (quantifier) and m/z 353.10→m/z 193.20 (qualifier); 15 keto-PGE2: m/z 349.30→m/z 331.20 (quantifier) and m/z 349.30-m/z 113.00 (qualifier); 13, 14-dihydro 15-keto PGE2: m/z 351.20→m/z 333.30 (quantifier) 10 and m/z 351.20→m/z 113.05 (qualifier); PGE2-D4: m/z 355.40→m/z 275.20; and PGF2α-D9: m/z 362.20→m/z 318.30. Dwell time was 20-30 ms.

Quantitative analysis was done using LabSolutions LCMS (Shimadzu). An internal standard method was used for quantification: PGE2-D4 was used as an internal standard for quantification of PGE2, 15-keto PGE2, and 13, 14-dihydro 15-keto PGE2. PGF2α-D9 was the internal standard for quantification of PGD2 and PGF2u. Calibration curves were linear (R>0.99) over the concentration range using a weighting factor of 1/X2 where X is the concentration. The back-calculated standard concentrations were ±15% from nominal values, and +20% at the lower limit of quantitation (LLOQ).

### In Vivo and In Situ Muscle Force Measurement

The peak isometric torque (N·mm) of the ankle plantarflexors was assessed as previously described (8,9). Briefly, the foot of anesthetized mice was placed on a footplate attached to a servomotor (model 300C-LR; Aurora Scientific). Two Pt-Ir electrode needles (Aurora Scientific) were inserted percutaneously were inserted subcutaneously over the tibial nerve, just posterior/posterior-medial to the knee. The ankle joint was secured at a 90° angle. The peak isometric torque was achieved by varying the current delivered to the tibial nerve at a frequency of 200 Hz and a 0.1-ms square wave pulse. We performed three tetanic measurements on each muscle, with 1 min recovery between each measurement. Data were collected with the Aurora Scientific Dynamic Muscle Data Acquisition and Analysis Software.

### Statistical Analyses

We performed cell culture experiments in at least three independent experiments where three biological replicates were pooled in each. We used a paired t-test for experiments where control samples were from the same experiment in vitro or from contralateral limb muscles in vivo. A non-parametric Mann-Whitney test was used to determine the significance difference between untreated vs treated groups using α=0.05. ANOVA or multiple t-test was performed for multiple comparisons with significance level determined using Bonferroni correction as indicated in the figure legends. Unless otherwise described, data are shown as the mean±s.e.m.

### REFERENCES

1. Chang J, et al. (2016) Clearance of senescent cells by ABT263 rejuvenates aged hematopoietic stem cells in mice. Nature Medicine 22(1):78-83).
2. A. Sacco et al., (2010) Short telomeres and stem cell exhaustion model Duchenne muscular dystrophy in mdx/mTR mice. Cell 143, 1059-1071.
3. Y. Zhang et al., TISSUE REGENERATION. Inhibition of the prostaglandin-degrading enzyme 15-PGDH potentiates tissue regeneration. Science 348, aaa2340 (2015).
4. C. Vinel et al., The exerkine apelin reverses age-associated sarcopenia. Nat Med 24, 1360-1371 (2018).
5. A. T. V. Ho et al., Prostaglandin E2 is essential for efficacious skeletal muscle stem-cell function, augmenting regeneration and strength. Proc Natl Acad Sci USA 114, 6675-6684 (2017).
6. D. J. Baker et al., Naturally occurring p16(Ink4a)-positive cells shorten healthy lifespan. Nature 530, 184-189 (2016).
7. J. K. Prasain, H. D. Hoang, J. W. Edmonds, M. A. Miller, Prostaglandin extraction and analysis in Caenorhabditis elegans. J Vis Exp, (2013).
8. E. L. Mintz, J. A. Passipieri, D. Y. Lovell, G. J. Christ, Applications of In Vivo Functional Testing of the Rat Tibialis Anterior for Evaluating Tissue Engineered Skeletal Muscle Repair. J Vis Exp, (2016).
9. K. A. Sheth et al., Muscle strength and size are associated with motor unit connectivity in aged mice. Neurobiol Aging 67, 128-136 (2018).

### Example 2. Inhibition of Prostaglandin Degrading Enzyme 15-PGDH Increases Muscle Strength in Aged Mice

### Introduction

With aging, a body-wide loss of muscle function diminishes quality of life and increases morbidity and mortality (1, 2). This disseminated muscle atrophy and loss of strength, or sarcopenia, accounts for $18 billion in annual healthcare costs in the United States alone (2). The identification of therapeutic agents for sarcopenia would be of major clinical benefit (1, 2).

During aging, skeletal muscles undergo structural and functional changes. The most apparent is loss of muscle strength, which in the lower body muscles can decline by 50-80% in aged humans, and is accompanied by a reduction in cross-sectional area of myofibers, muscle mass and strength (3). This loss of function arises from disrupted cell-cell interactions and aberrant cell signaling pathways, particularly those related to inflammation, protein turnover, and mitochondrial function (1, 4-6). Due to this multifactorial etiology, untangling causal molecular pathways in order to identify therapeutic targets to prevent, delay or reverse sarcopenia has proven challenging.

Previously, we determined that in young mice PGE2 stimulates muscle stem cells (MuSCs) and is essential to the regeneration of damaged muscles (7), in good agreement with findings regarding its function in the regeneration of bone, colon, liver, and blood (8-10). We reasoned that in aging, prostaglandin signaling might go awry. Using liquid chromatography coupled to atmospheric pressure ionization tandem mass spectrometry (LC-MS/MS) to distinguish closely related prostaglandin family members (11) we found that PGE2 and PGD2 levels are reduced in aged skeletal muscles.

We hypothesized that the decrease in prostaglandins in aged muscles might be due to increased prostaglandin catabolism by 15-hydroxyprostaglandin dehydrogenase (15-PGDH). Here, we uncover that elevated 15-PGDH is a hallmark of aged muscles and certain other aging tissues. Further, we show that in aged mice inhibition of 15-PGDH augments muscle mass and strength. Genetic experiments demonstrate that the beneficial effects of 15-PGDH inhibition are specific to increased PGE2 signaling. Our findings provide fresh insights into sarcopenia and suggest an innovative treatment strategy.

### Increase in Prostaglandin Degrading Enzyme (15-PGDH) in Aged Tissues

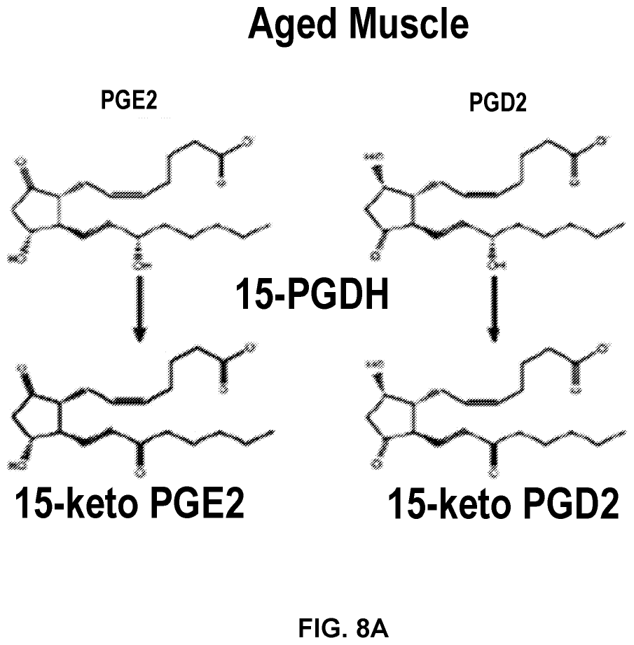

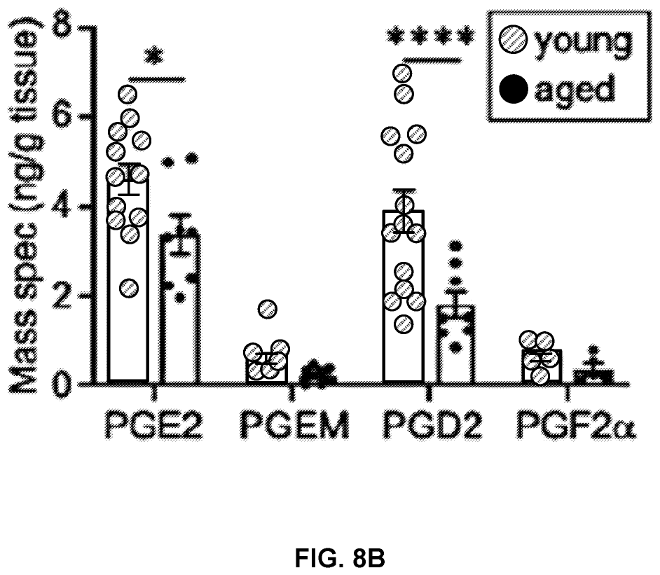

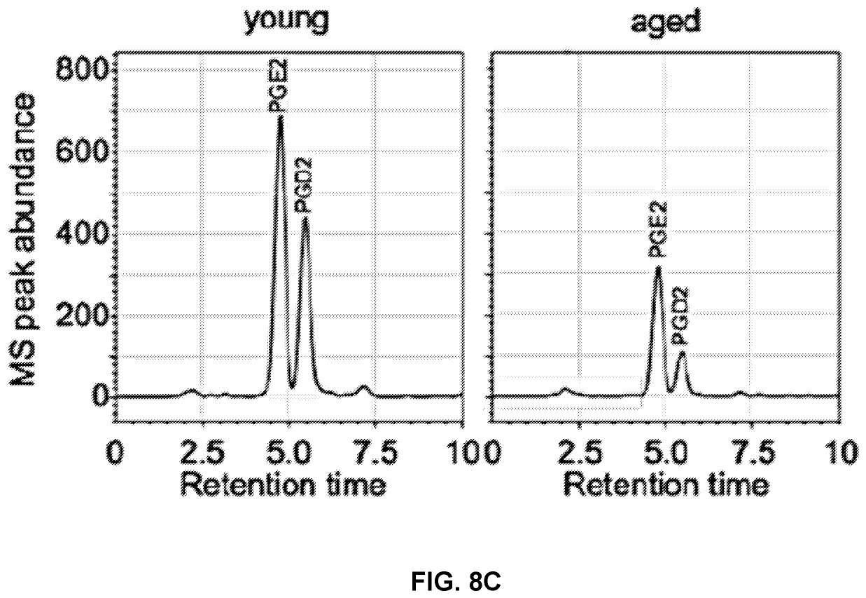

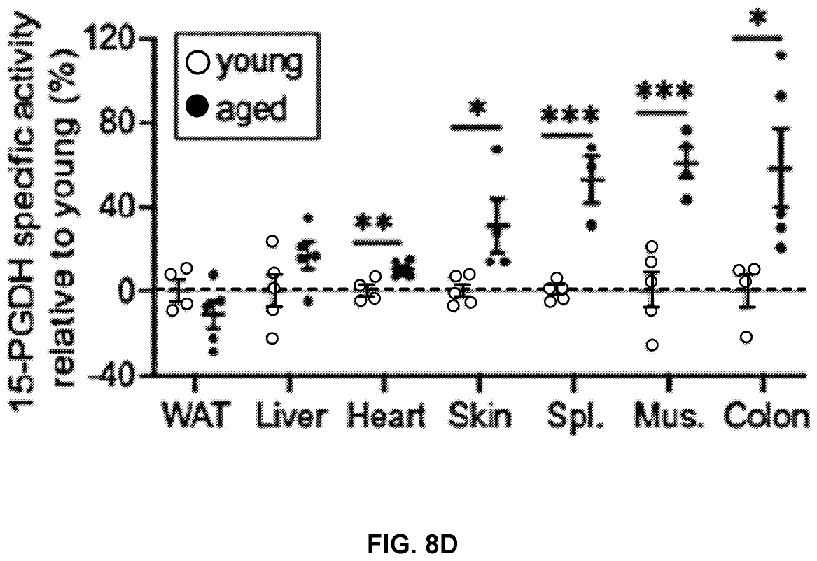

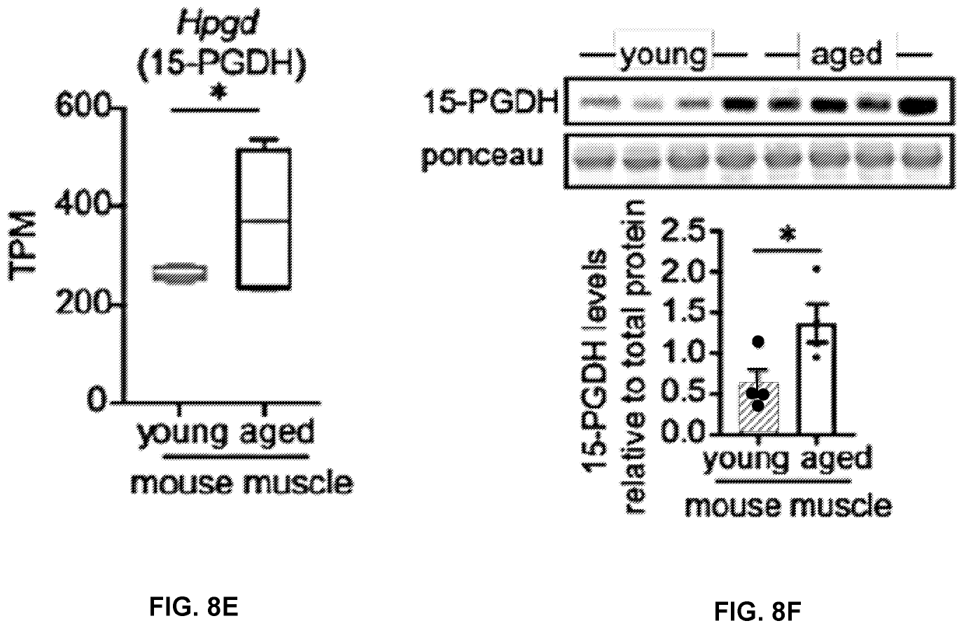

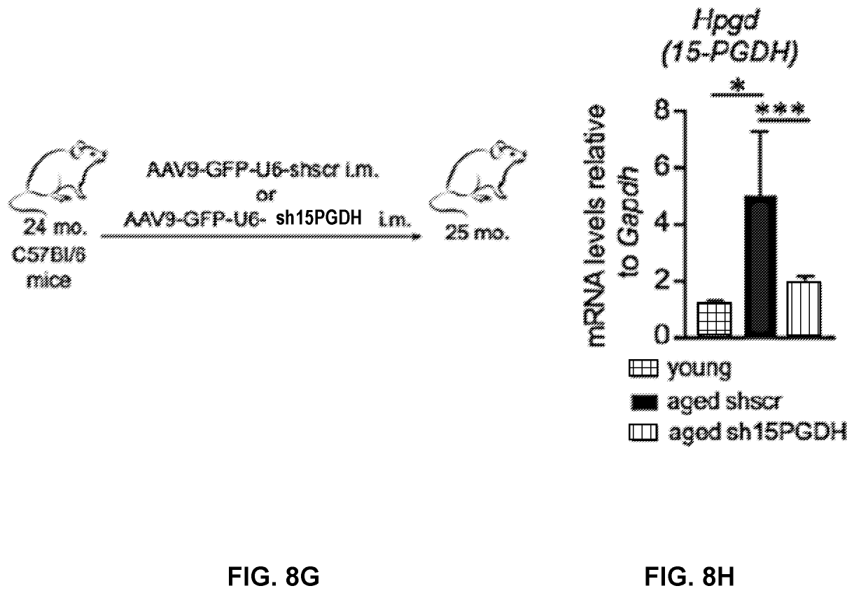

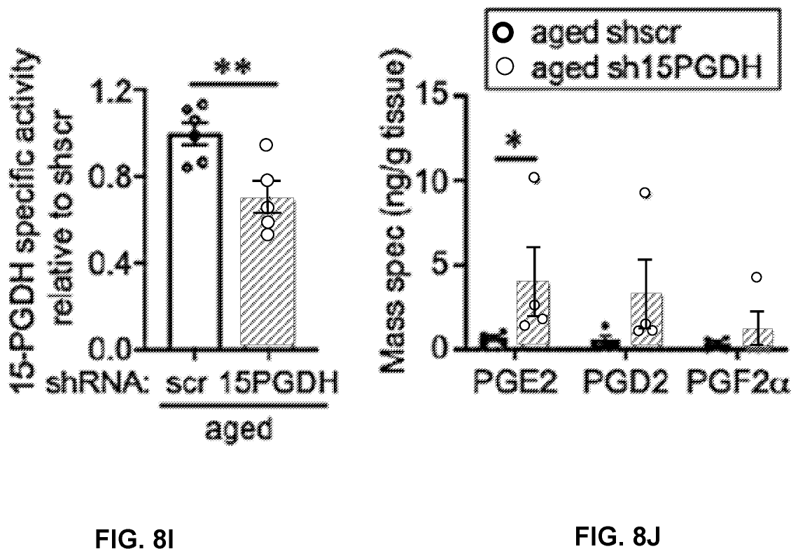

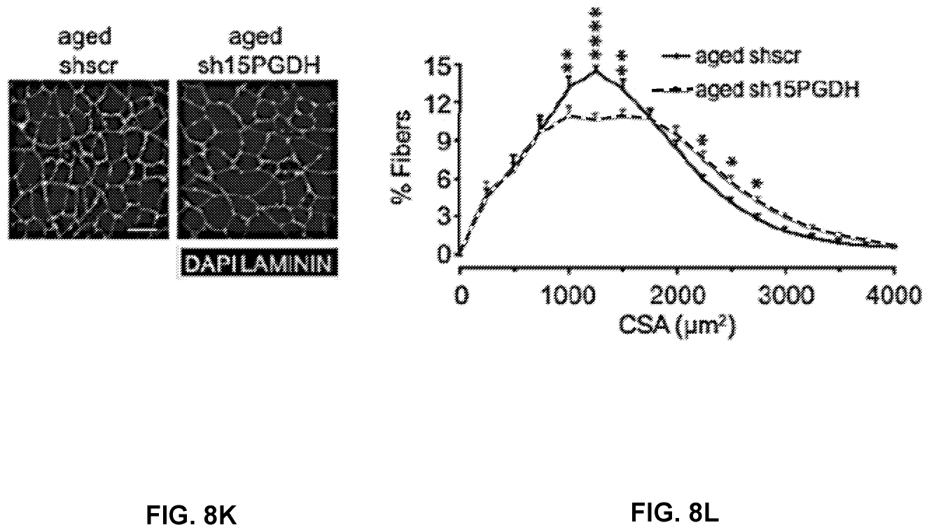

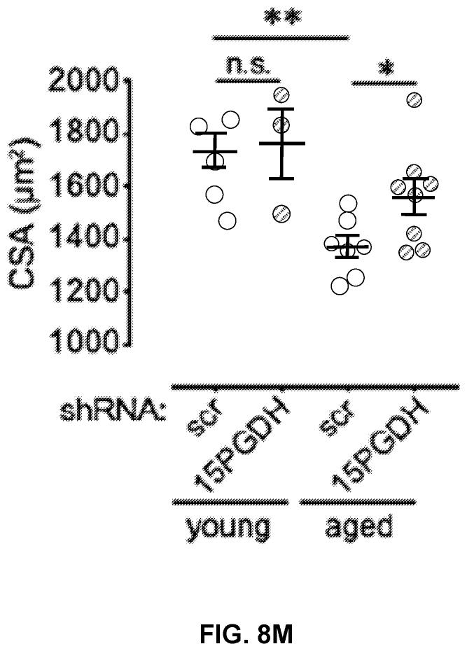

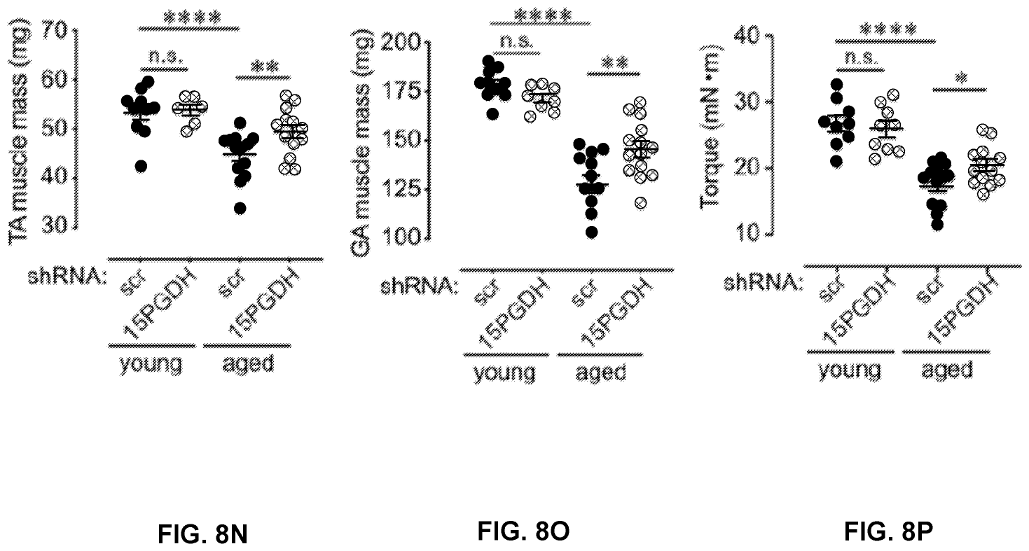

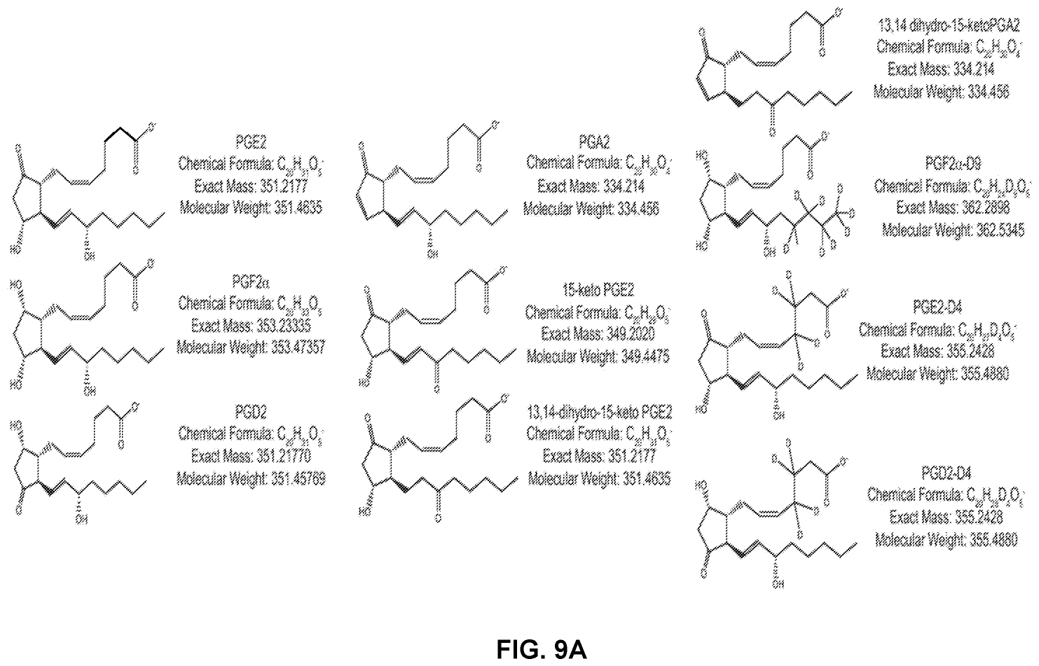

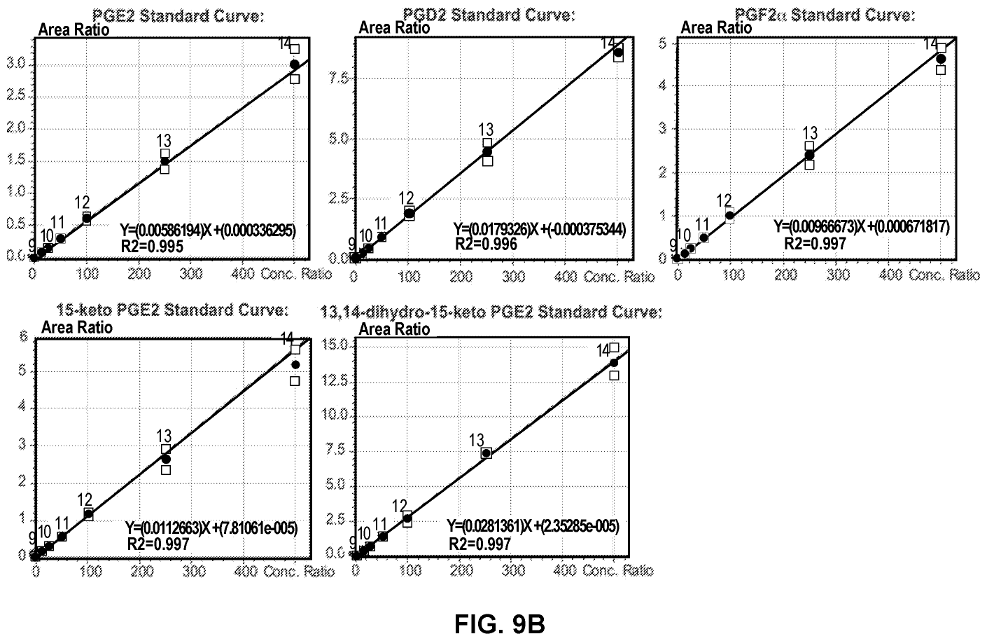

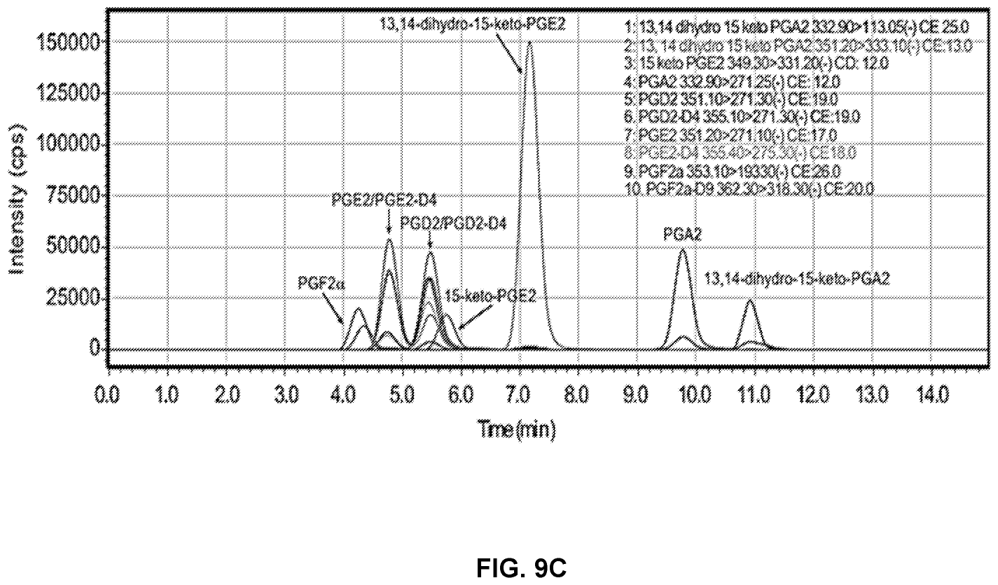

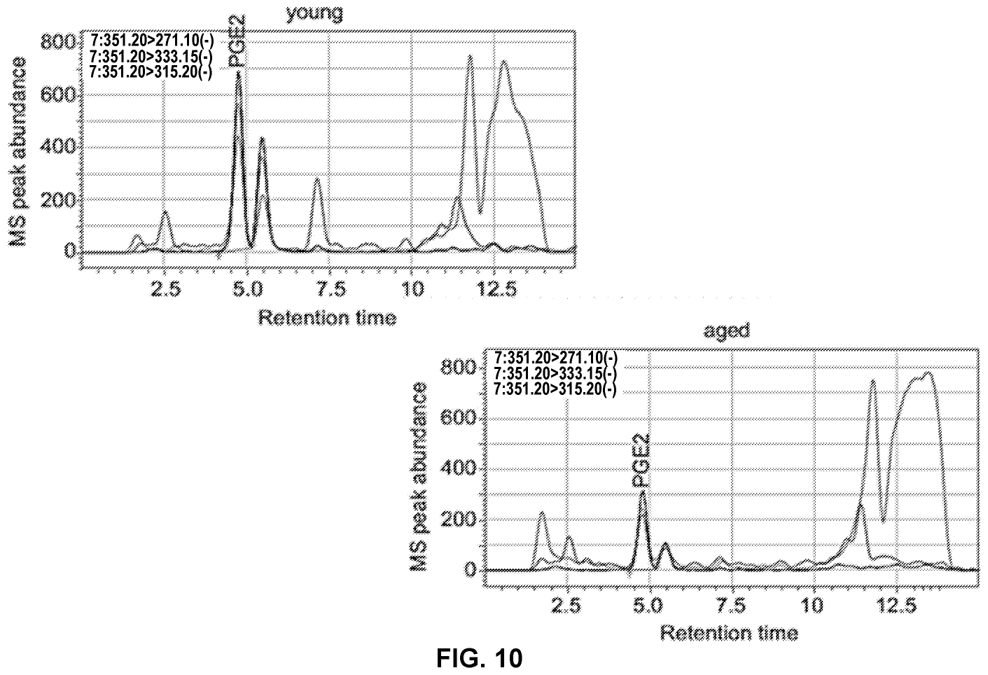

We previously demonstrated the importance of PGE2 signaling in stimulating stem cells to regenerate damaged tissues in young mice (7). We reasoned that PGE2 might also act on mature muscle myofibers and play a crucial role in the maintenance of muscle tissue homeostasis. We postulated that in aging, a decrease in PGE2 and other endogenous eicosanoids, lipid metabolites generated from membrane fatty acids, might occur and have deleterious effects on muscle tissue function. To analyze the eicosanoid composition of aged skeletal muscle, we used LC-MS/MS. This method overcomes the cross-reactivity of antibody-based assays, such as ELISAs and exceeds other mass spectrometry methods in its resolution of related eicosanoids of the same mass, PGE2 and PGD2, as well as PGF2a (FIGS. 8A-C, 9A-C, and 10A). This is achieved by isolating and homogenizing the hindlimb muscles from young and aged mice followed by acetone precipitation to exclude proteins. A 2-step liquid-liquid extraction is then performed to enhance LC-MS/MS sensitivity. We observed a significant decline in PGE2 and PGD2 levels in aged muscles (FIGS. 8A-C, 9A-C, and 10A). PGE2 and PGD2 are degraded by a multi-step process initiated by the rate-limiting enzyme 15-PGDH to yield the unstable 15-keto-PGE2 and 15 keto-PGD2 metabolites which are then converted to multiple downstream metabolites, including the 13,14-dihydro-15-keto-PGE2 metabolite (PGEM) (12, 13). These intermediates were either not detected at all or only at low levels by LC-MS/MS due to their instability (FIGS. 8C and 9C). The MS spectral plots demonstrate that this method readily distinguishes among closely related eicosanoids.

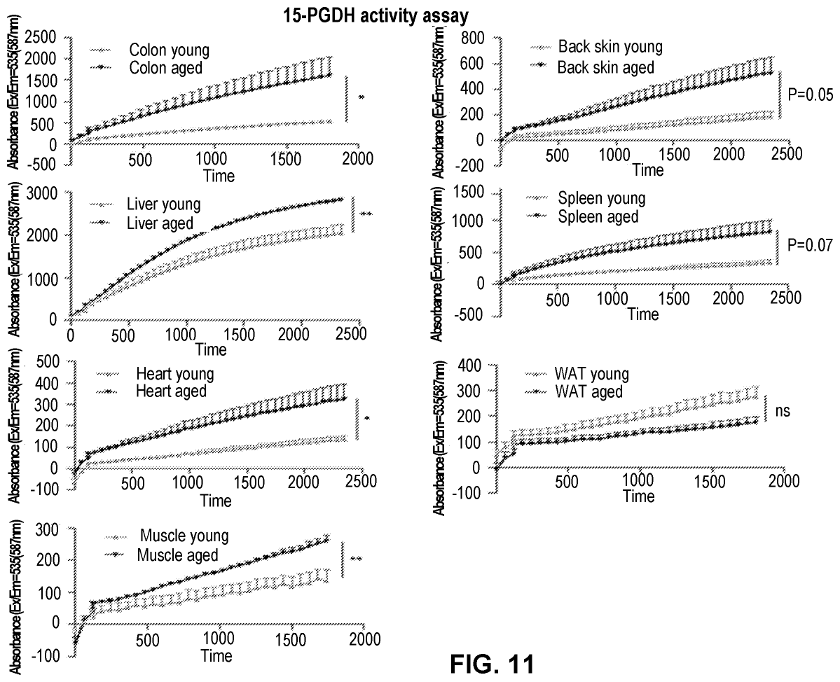

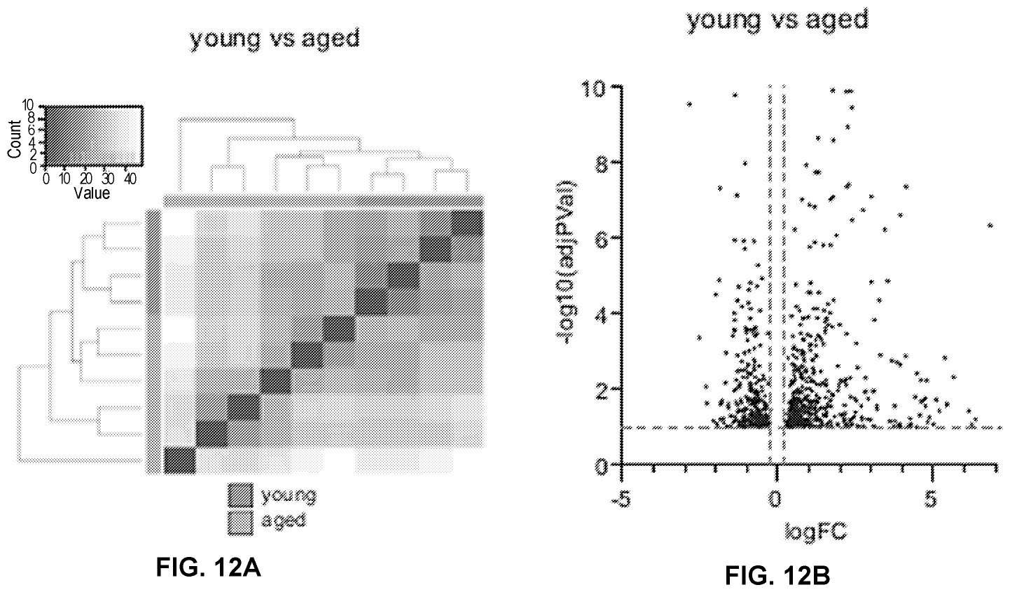

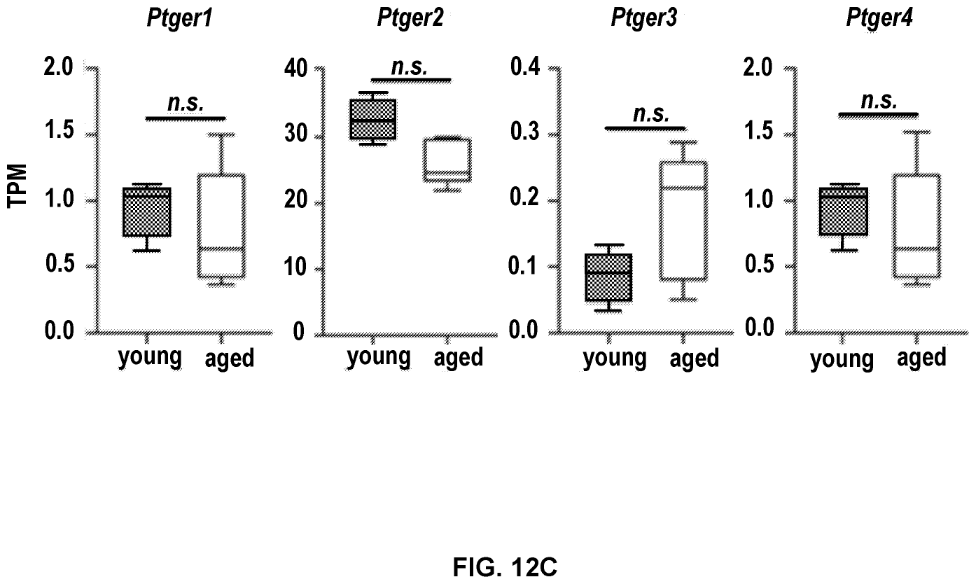

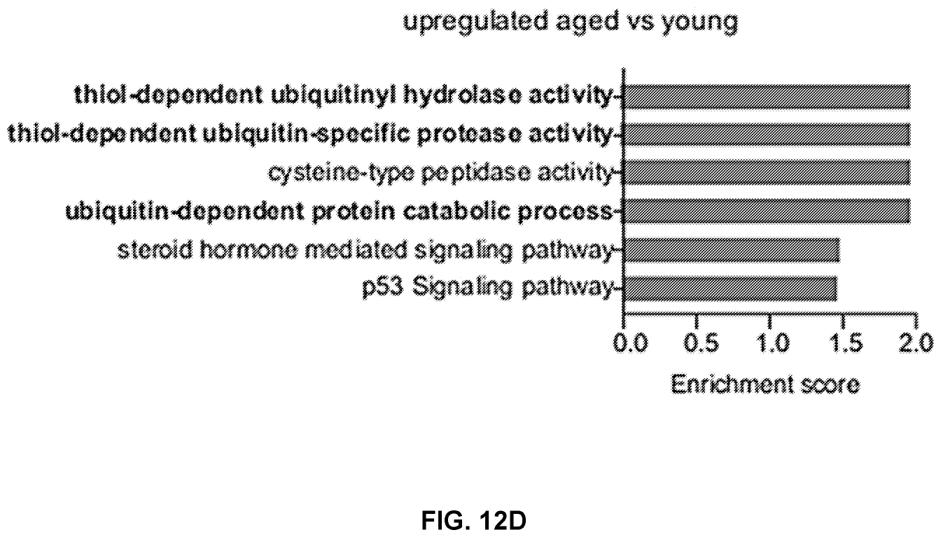

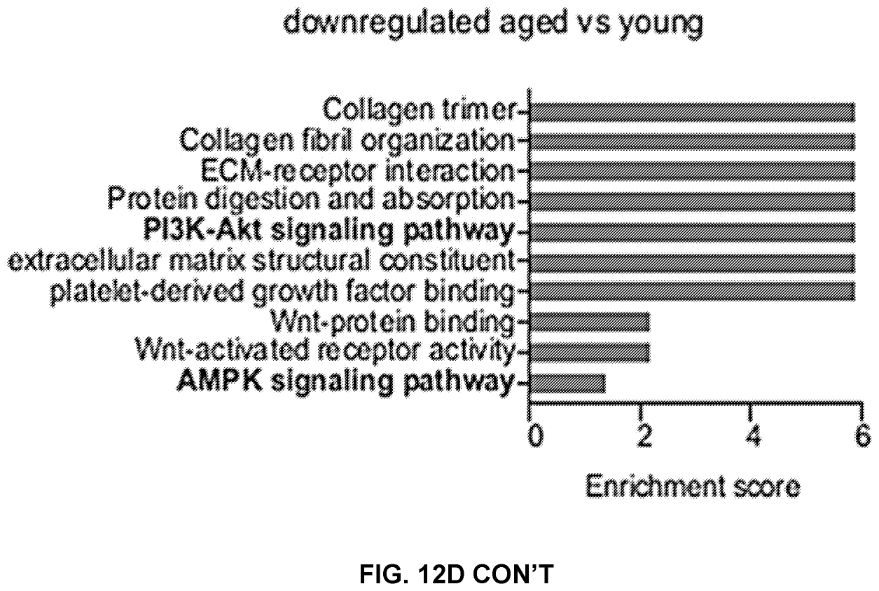

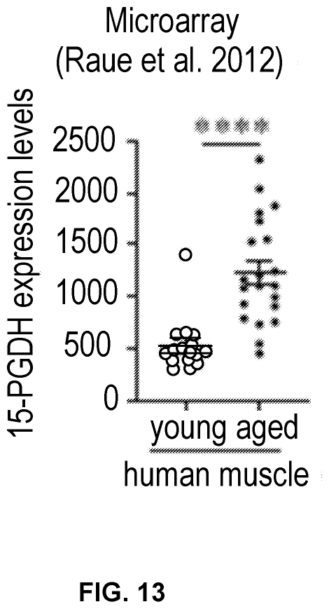

We hypothesized that an increase in the degrading enzyme 15-PGDH could account for the observed reduction in PGE2 and PGD2 in muscle and might constitute a general characteristic of aged tissues. In agreement, we found that the specific activity of the enzyme was elevated not only in aged skeletal muscles, but also in aged cardiac, skin, spleen, and colon tissues (FIGS. 8D and 11). Accordingly, 15-PGDH mRNA and protein are significantly increased in aged muscles (FIGS. 8E, 8F, 12A, and 12B). To determine the relevance of this finding to human aging, we reanalyzed publicly available microarray data for young and aged human muscle samples (14) and found that expression of 15-PGDH was significantly increased in aged human (78±6 yrs) biopsies from the vastus lateralis muscle compared to those from young populations (25±3 yrs) (FIG. 13A). Together, these data identify 15-PGDH as a potential driver of the decline in prostaglandin levels seen in aged muscle.

### Increase in Aged Muscle Mass and Strength Following Inhibition Of 15-PGDH

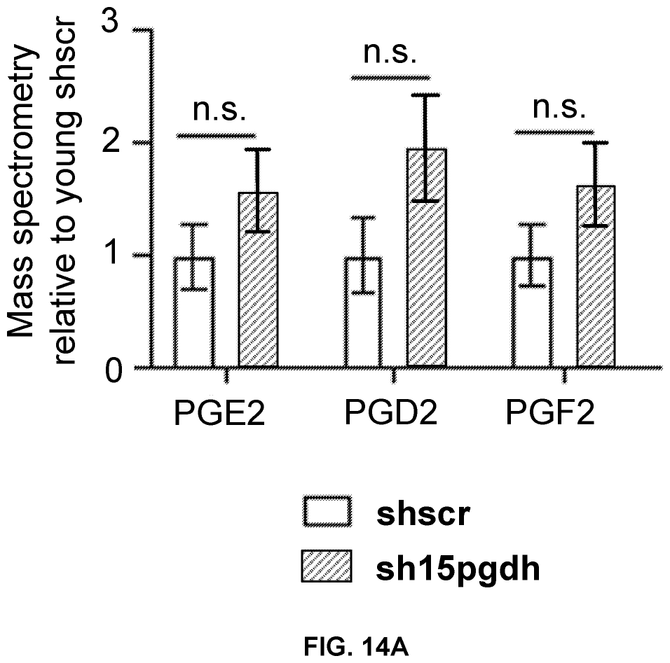

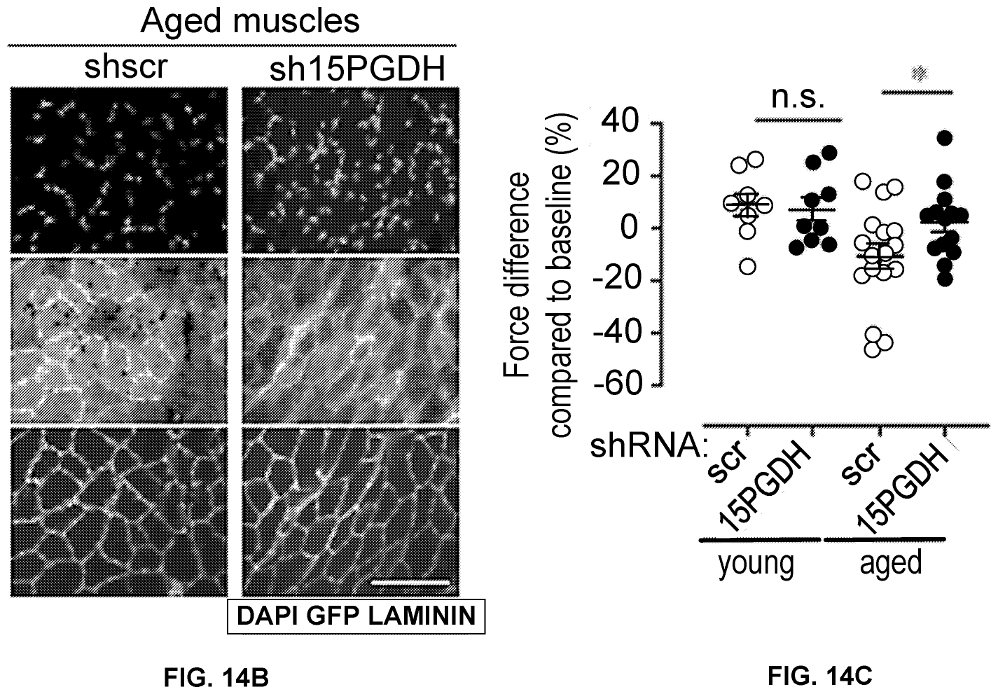

We postulated that inhibition of 15-PGDH could lead to increased levels of PGE2 and PGD2 which in turn could ameliorate muscle wasting in aged mice. Like humans, aged mice exhibit sarcopenia, a general loss of muscle strength (1). We first used a genetic approach to reduce enzyme levels that entailed adeno-associated virus (AAV9) intramuscular (i.m.) delivery of either GFP and shRNA to 15-PGDH or control AAV9 encoding GFP and a scrambled (scr) shRNA under the control of a ubiquitous promoter (U6) (FIG. 8G). The resulting localized intramuscular gene therapy delivery strategy led to a significant reduction in 15-PGDH mRNA levels and specific activity and an increase in PGE2 and PGD2 levels assessed by mass spectrometry (FIGS. 8H-J and 14A). That these vectors targeted muscle was confirmed by immunofluorescence analysis of the GFP reporter in transduced Tibialis anterior (TA) and Gastrocnemius (GA) muscles (FIG. 14B). Genetic knockdown of 15-PGDH in aged, but not young, muscles was accompanied by a marked increase in cross-sectional myofiber area in 15-PGDH shRNA treated aged muscles compared to controls (FIGS. 8K-M). Furthermore, in contrast to young, knockdown of 15-PGDH in aged muscles resulted in a significant increase in both muscle mass and muscle force one month after treatment (FIGS. 8N-P and 14C).

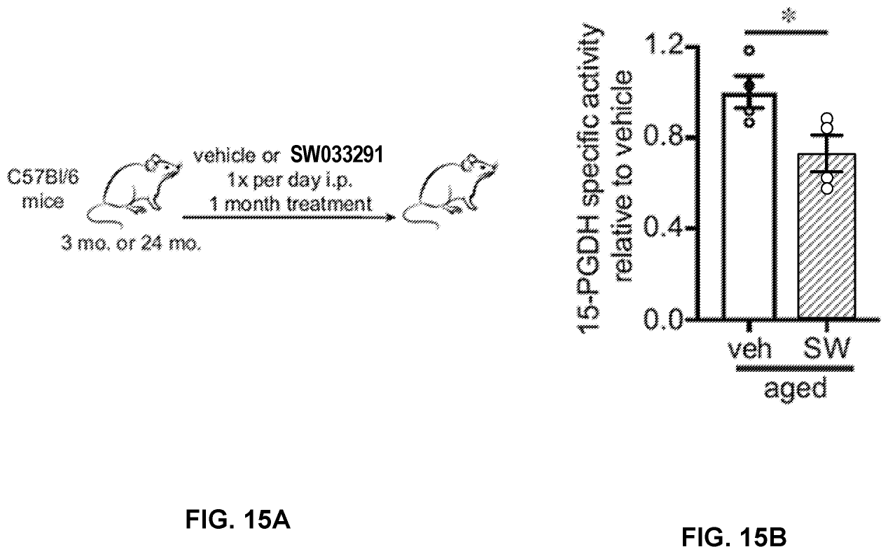

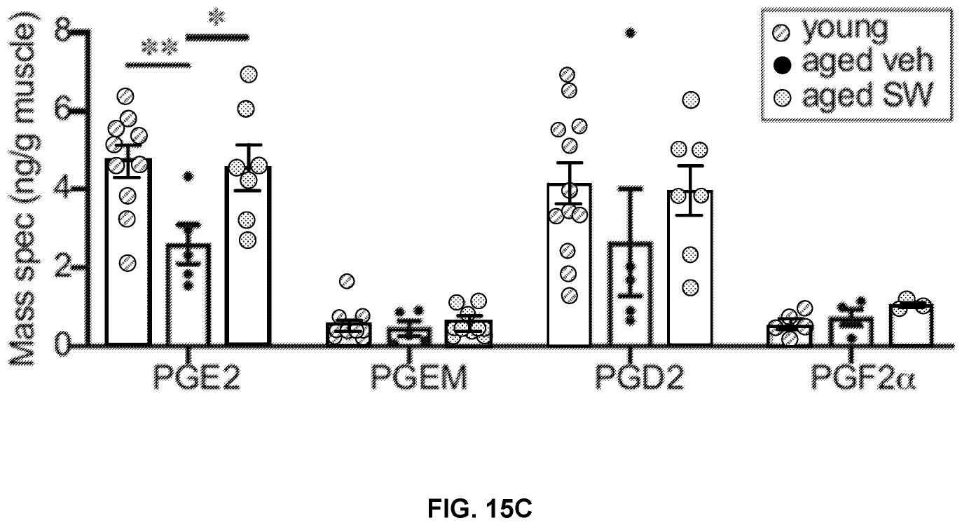

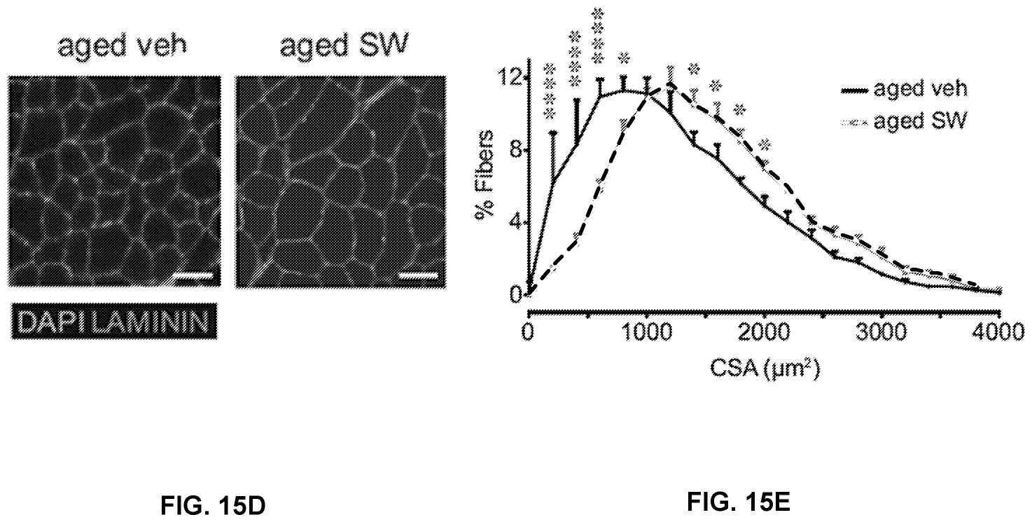

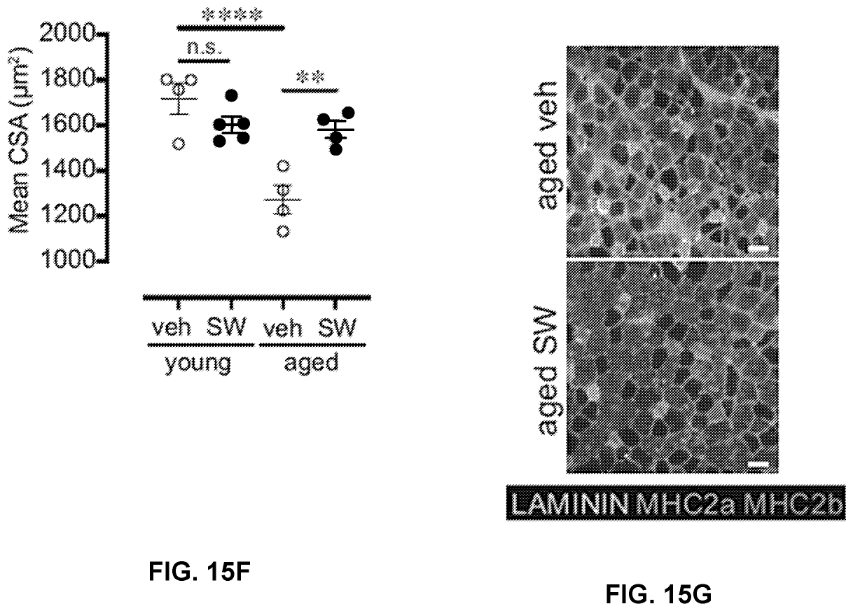

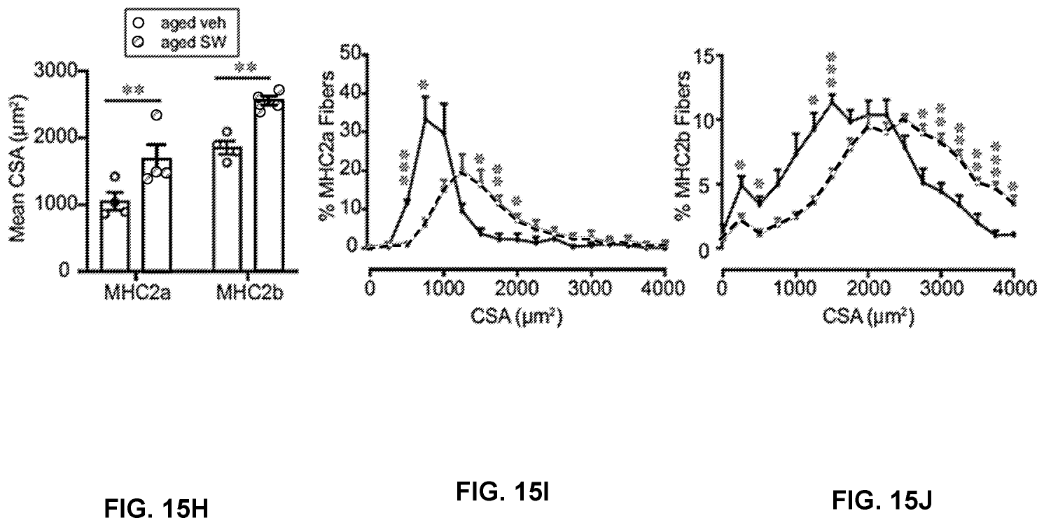

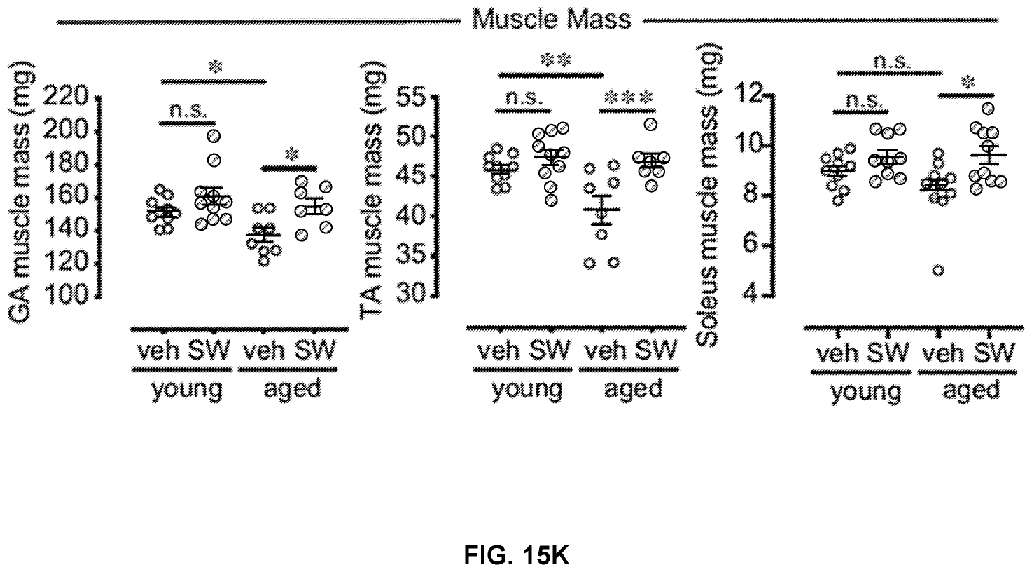

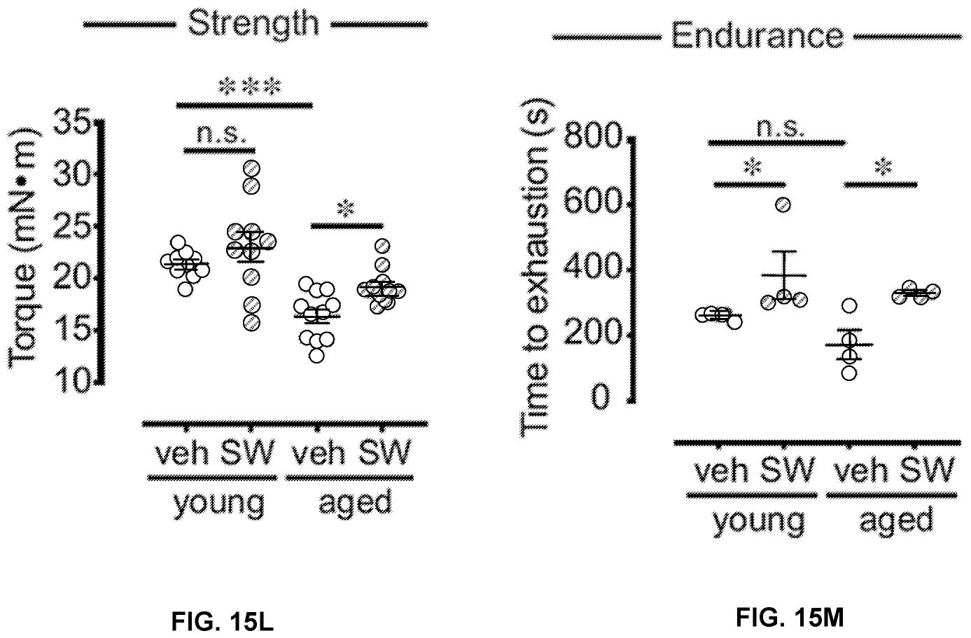

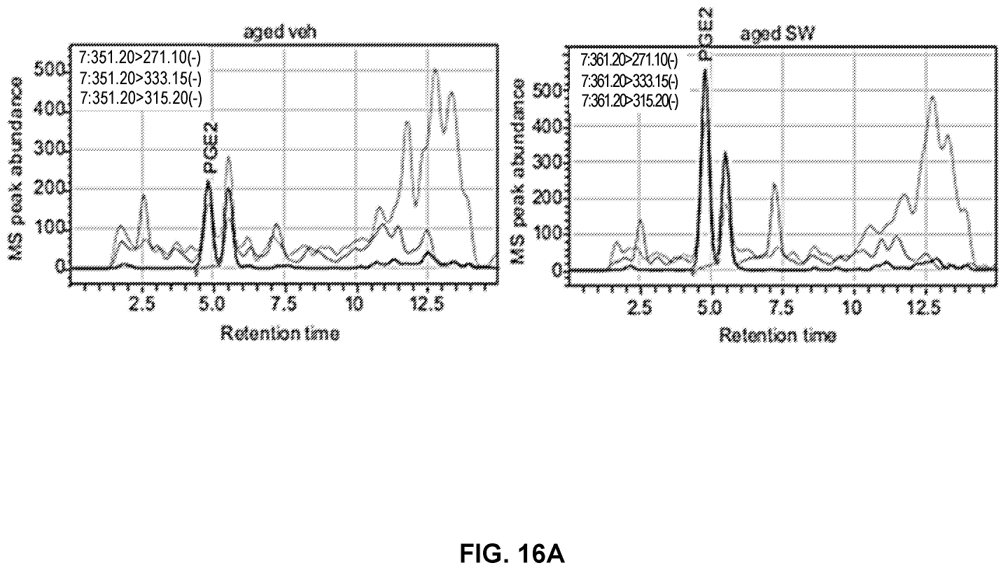

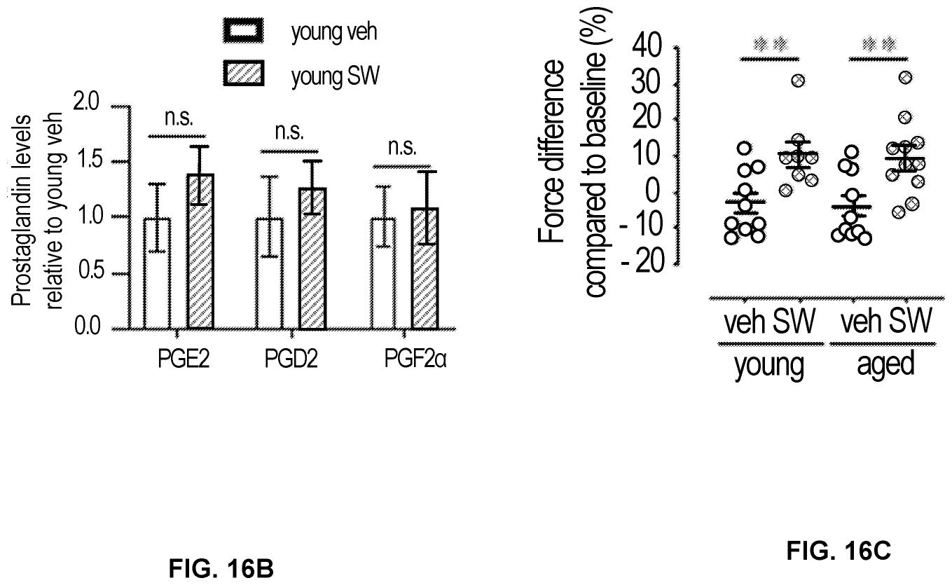

To test if the disseminated muscle wasting seen in sarcopenia could be overcome by systemic delivery of a small molecule inhibitor of 15-PGDH, we treated aged mice and young control mice intraperitoneally with SW033291 (SW) or vehicle (10) (FIG. 15A). SW was previously extensively characterized as a specific inhibitor of 15-PGDH that is noncompetitive with PGE2 with an apparent Ki of 0.1 nM (10). In vivo, SW was previously shown to increase PGE2 levels 2-fold, and to a lesser extent PGD2 levels, in bone marrow, colon, lung, and liver, which augmented regeneration following injury of these tissues in young mice (10). We found that after one month of daily intraperitoneal SW treatment, 15-PGDH specific activity was significantly reduced in aged muscles and a concomitant increase in the levels of PGE2 and PGD2 was detected by LC-MS/MS that was on par with young muscles (FIGS. 15B, 15C, 16A, and 16B). Histological analysis revealed that myofiber cross-sectional area was significantly augmented in SW-treated aged mice but not in young, indicating that muscle atrophy in the aged was attenuated (FIGS. 15D-F). Fiber type analysis revealed that SW treatment promoted an increase in the cross-sectional area of both oxidative (type IIa) and glycolytic (type IIb) fibers (FIGS. 15G-J). SW-treated young mice exhibited a trend toward increased muscle mass and absolute strength that was not statistically significant (FIGS. 15K, 15L, and 16C). In contrast, SW-treated aged mice exhibited a significant increase in mass of TA, GA and soleus muscles (FIG. 15K) and in plantar flexor muscle force (FIGS. 15L and 16C). Moreover, endurance (time to exhaustion on a treadmill) was increased, suggestive of an overall systemic beneficial effect in addition to muscle strength (FIG. 15M). Taken together, our studies using the small molecule inhibitor, SW, corroborate our findings using a genetic loss of function via a localized shRNA and show that a decrease of 15-PGDH activity systemically for a period of one month suffices to attenuate skeletal muscle atrophy and augment muscle function in aged mice.

### 15-PGDH Expression by Senescent Interstitial Cells in the Aged Muscle Microenvironment

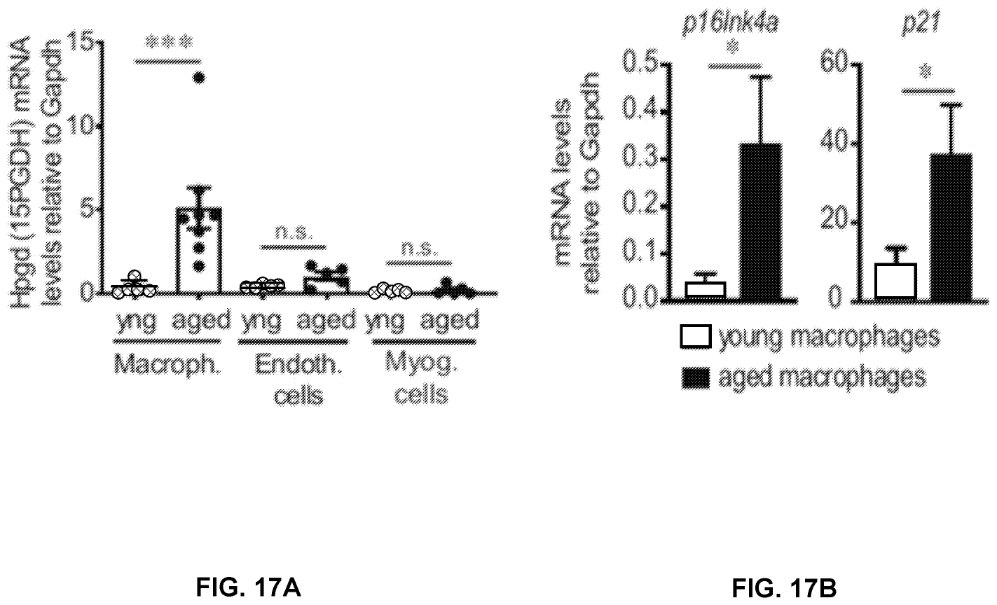

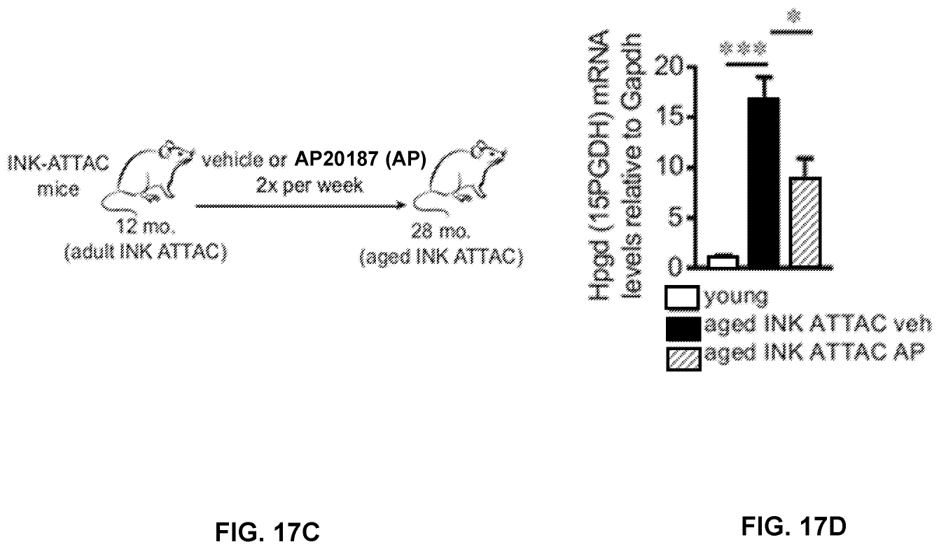

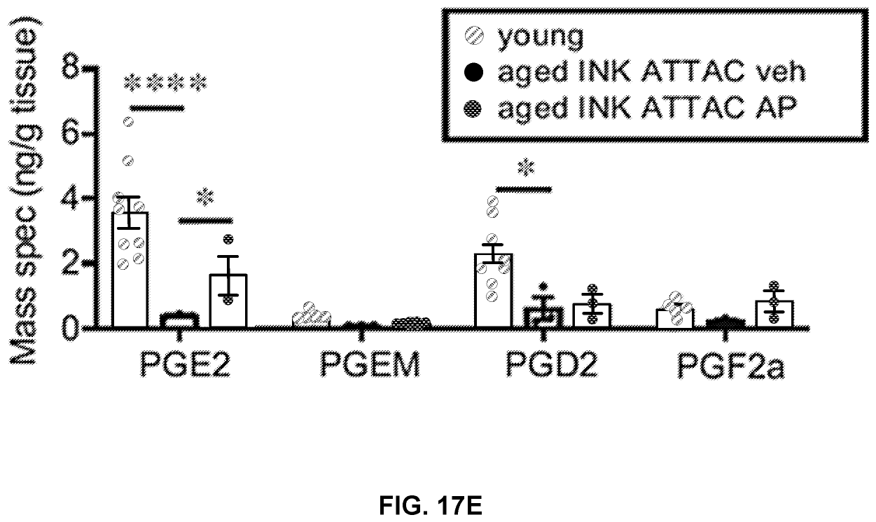

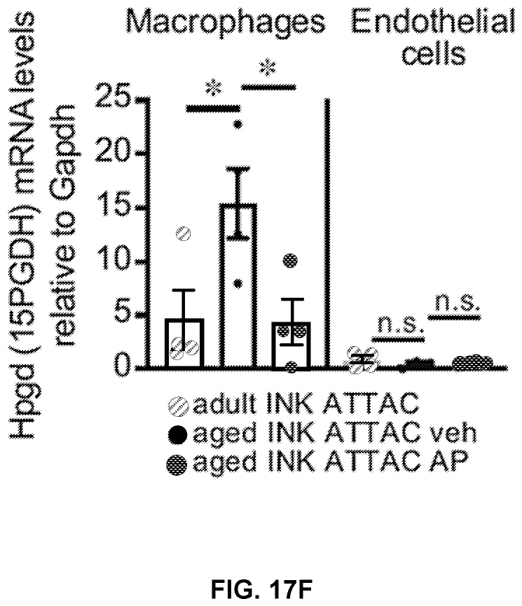

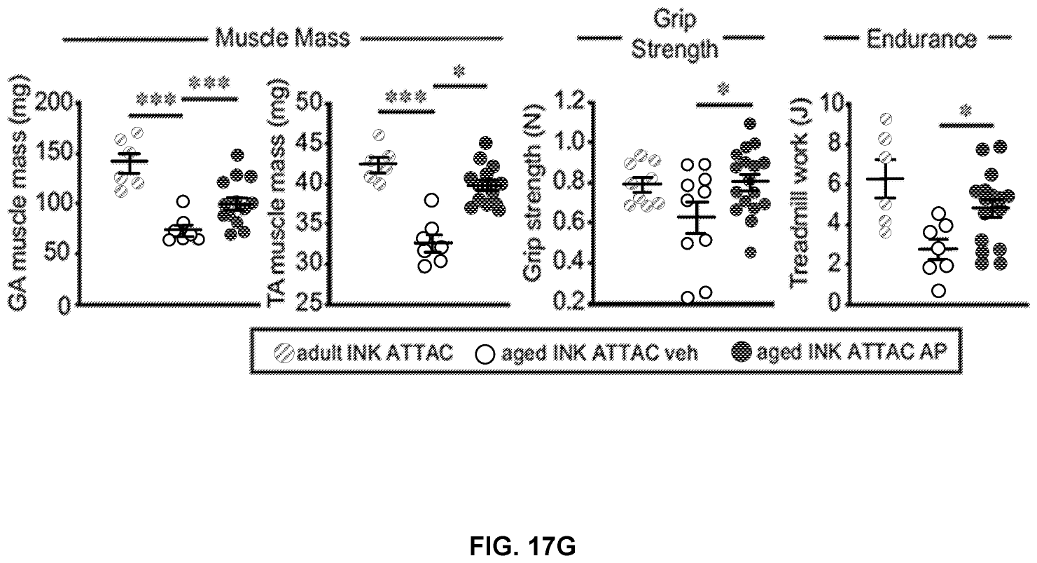

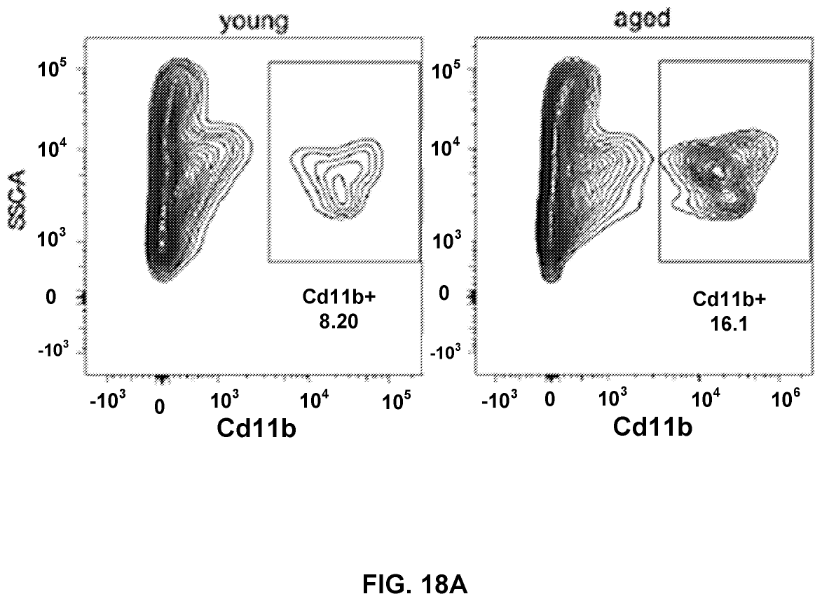

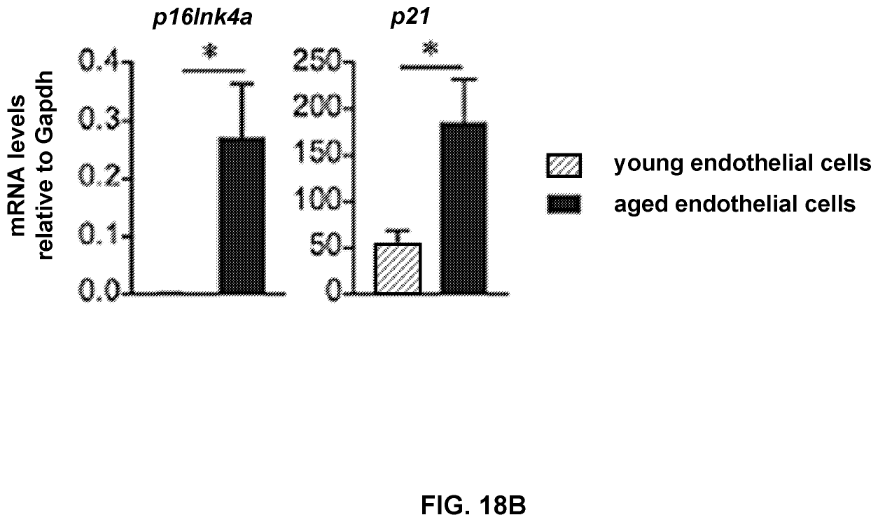

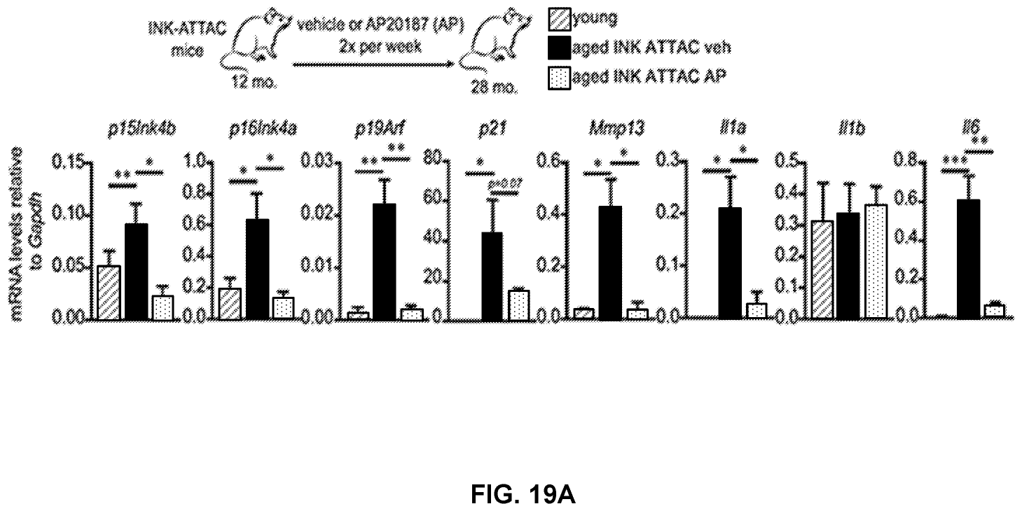

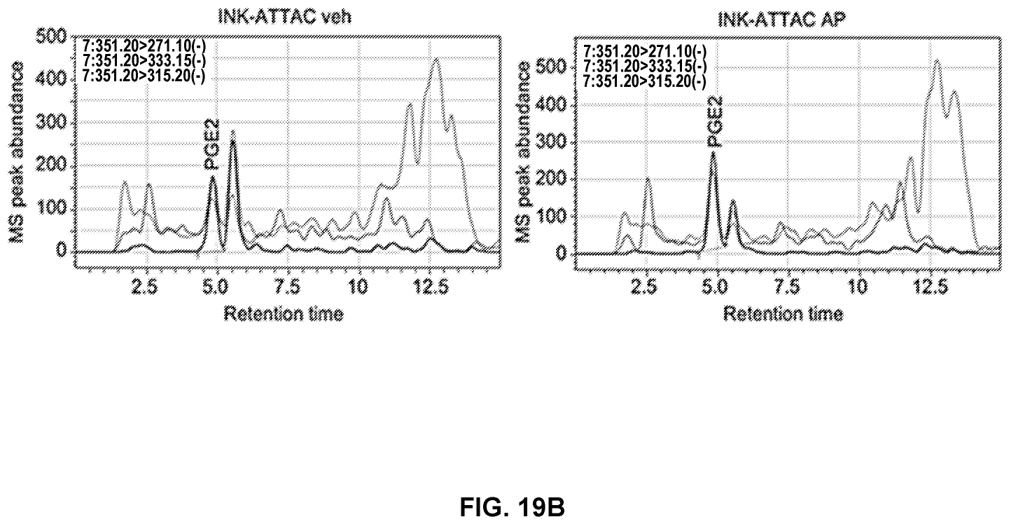

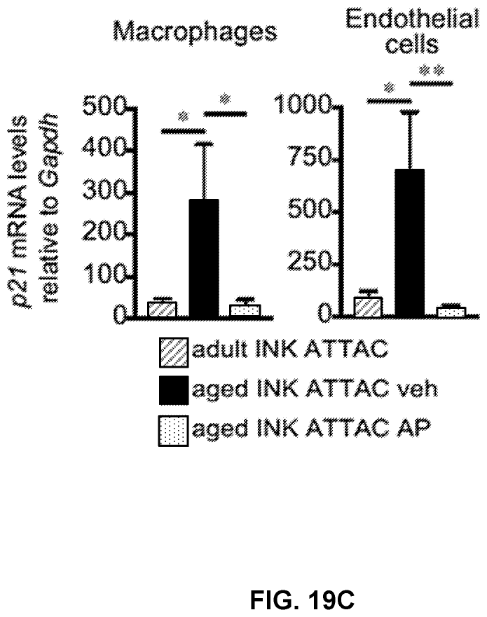

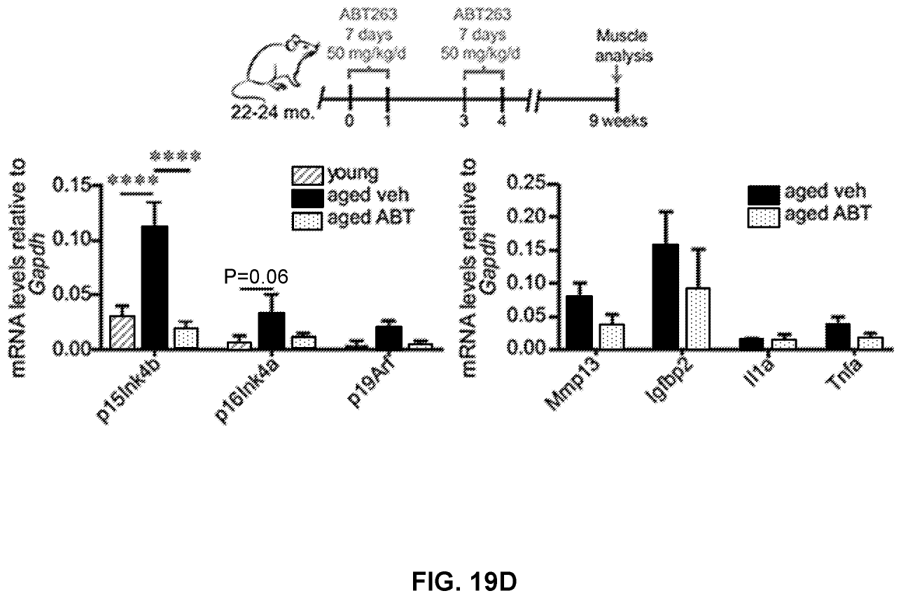

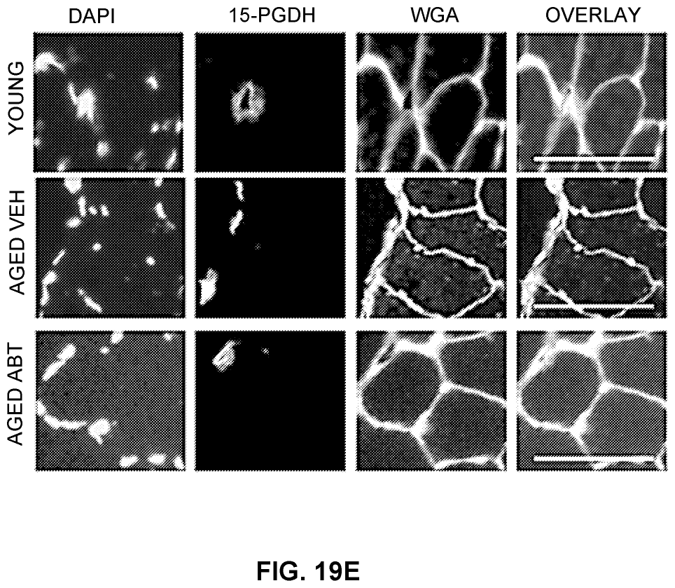

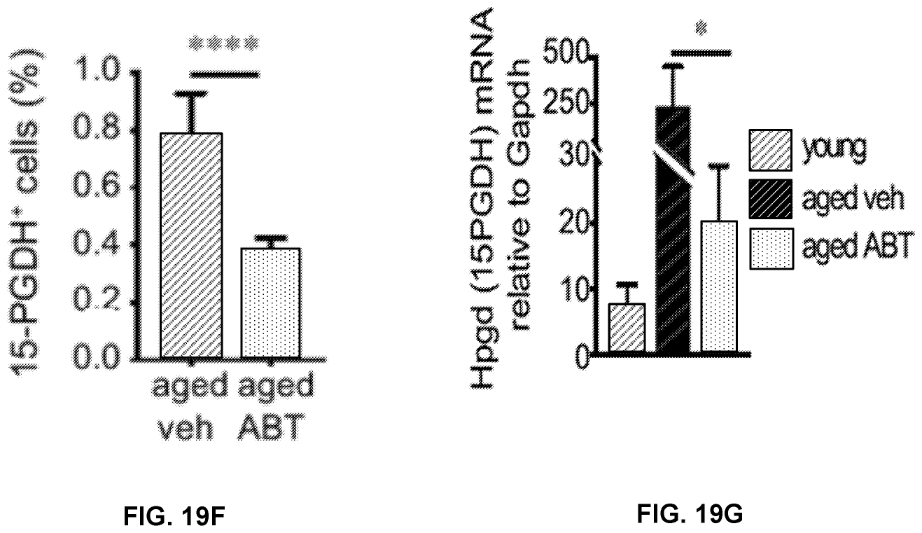

We sought to identify the cell source of 15-PGDH in aged muscle tissue. To this end, we analyzed Hpgd (15-PGDH) mRNA levels in cells isolated by fluorescence activated cell sorting from dissociated young and aged muscle tissues. A striking increase in 15-PGDH transcript levels was detected in FACS purified macrophages (Cd11b+/Cd11c-/F4/80+/Cd31-), but not in endothelial (Cd31+/Cd11b-/Cd11c-/F4/80-) or myogenic stem and progenitor cells (a7+/Cd11b-/Cd45-/Cd31-/Scal-) isolated from aged muscles (FIGS. 17A, 18A, and 18B). Additionally, aged macrophages and endothelial cells expressed high levels of cell cycle regulators p16 (Ink4a, Cdkn2a) and p21 (Cdkn1a) (FIGS. 17B and 19C), which are markers of senescent cells that have been reported to accumulate and adversely affect tissue function with aging, including muscle (15). To determine if senescent cells were the source of 15-PGDH in aged muscles, we utilized two strategies to ablate these cells, a genetic model and a senolytic drug treatment. First, we analyzed muscles from INK-ATTAC transgenic mice in which senescent cells are cleared by expression of a FK506-binding-protein-caspase8 fusion protein under the control of a minimal Ink4a promoter (p16) in response to treatment with AP20187 (AP), a dimerizer that activates the fusion protein leading to cell death (16) (FIGS. 17C and 19A). Following a 16-month AP treatment of aged INK-ATTAC mice, 15-PGDH transcript levels were markedly reduced (FIG. 17D), which led to an increase in PGE2 levels analyzed by LC-MS/MS (FIGS. 17E and 19B). To determine the cell source of 15-PGDH in this mouse model, we FACS isolated macrophages from control and AP-treated INK-ATTAC muscles and found reduced levels of 15-PGDH in these cells after clearance of senescent cells (FIG. 17F), in accordance with the reduced expression of p16 and p21 (FIG. 19C). In contrast, FACS isolated senescent endothelial cells did not express significant 15-PGDH levels (FIGS. 17F and 19C). Notably, myofibers do not die and their function is improved. Elimination of senescent cells in aged mice led to an increase in hindlimb muscle mass (TA and GA), strength assessed as grip strength, and endurance assessed as a composite measure of distance run on a treadmill until exhaustion and body weight (FIG. 17G). These aged mice in which senescent cells had been ablated, ran for a longer distance and had increased body mass, indicative of a higher capacity for work (FIG. 17G).

As a second approach, we induced apoptosis in aged senescent cells by treating aged mice with a senolytic agent, ABT-263, also known as navitoclax, a pan-Bc1 inhibitor (17) (FIG. 19D). After two months of treatment, the percentage of cells expressing 15-PGDH detected by immunohistochemistry and overall 15-PGDH gene expression levels detected by qRT-PCR in muscle tissues were markedly decreased (FIGS. 19D-G). Muscle tissue resident interstitial cells that exhibited the highest 15-PGDH staining were ablated by this senolytic treatment (FIGS. 19E and 19F), whereas myofibers were spared. These results suggest that PGE2 is degraded, in part, by a paracrine mechanism whereby senescent 15-PGDH-expressing interstitial cells, such as macrophages, in the vicinity of myofibers degrade PGE2 and contribute to the dysfunction of the aged myogenic niche, or microenvironment.

Reduced Muscle Strength after Ectopic Expression of 15-PGDH in Young Muscles

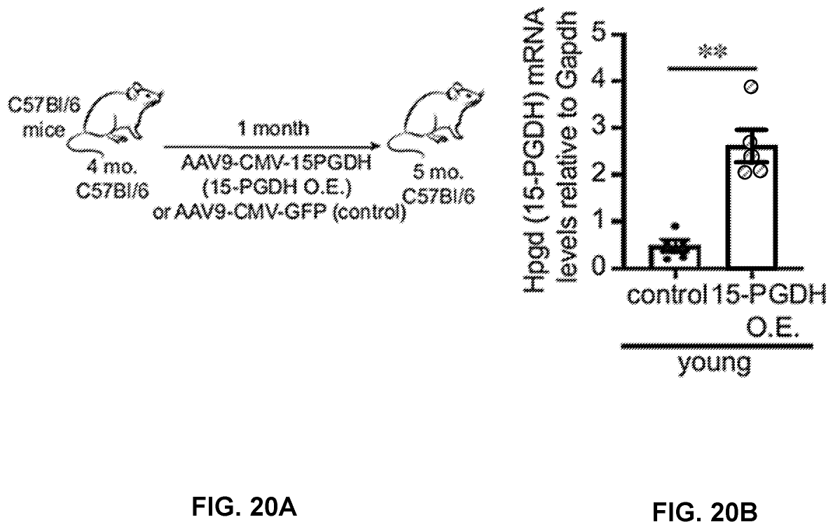

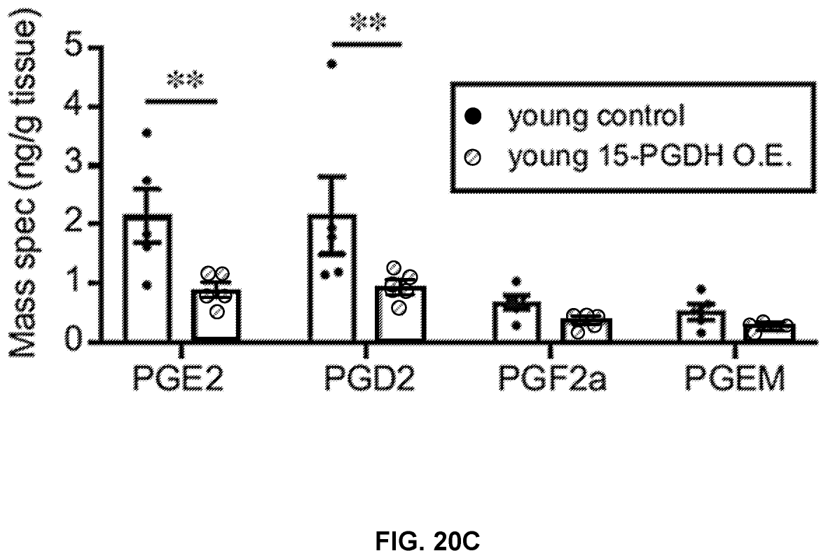

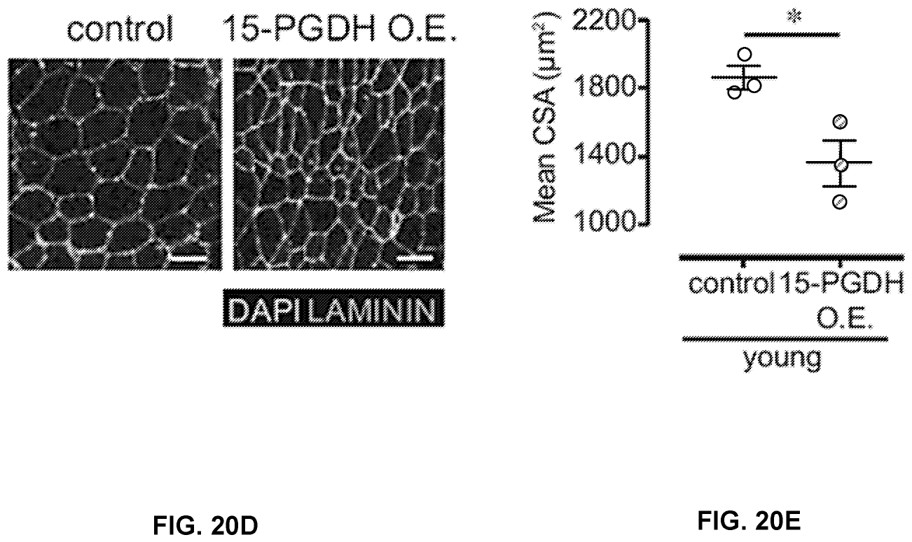

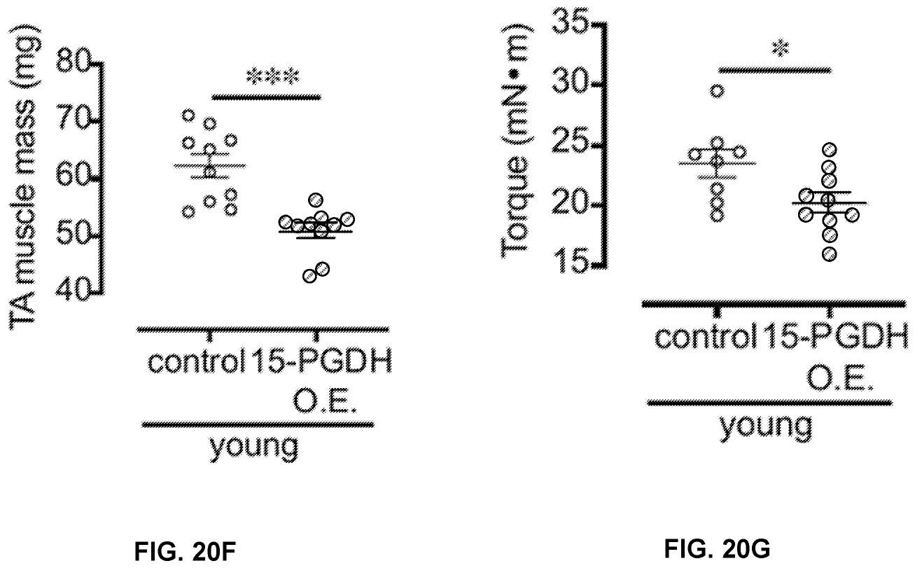

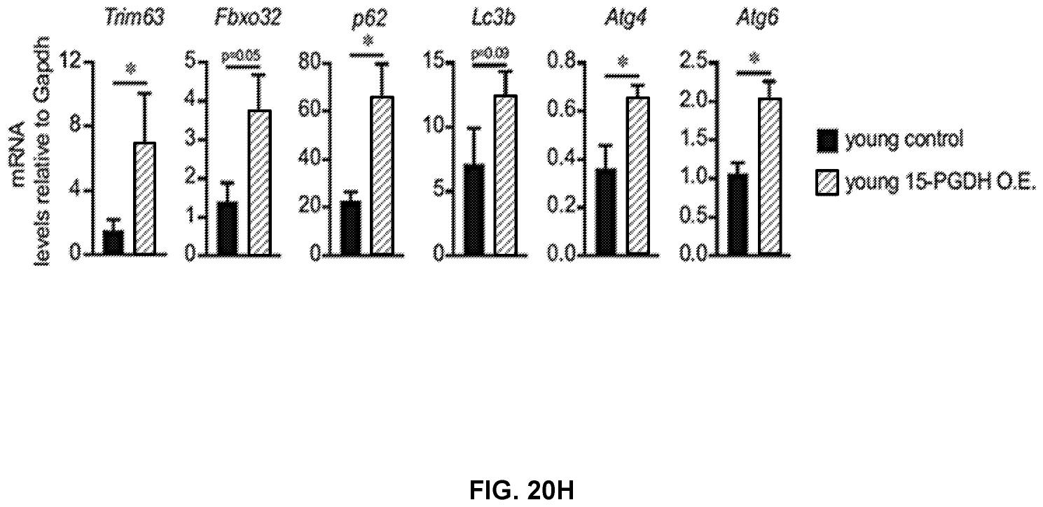

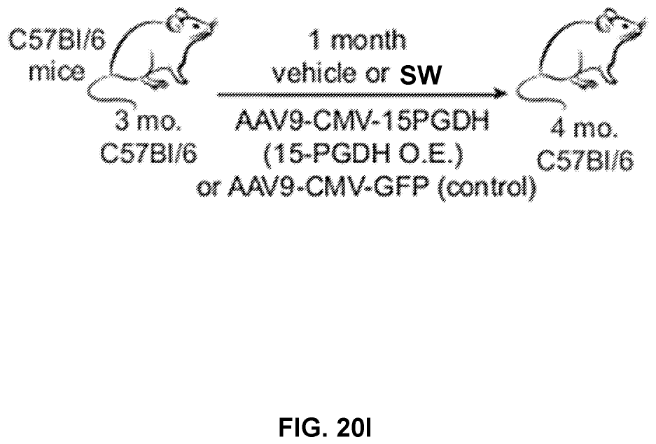

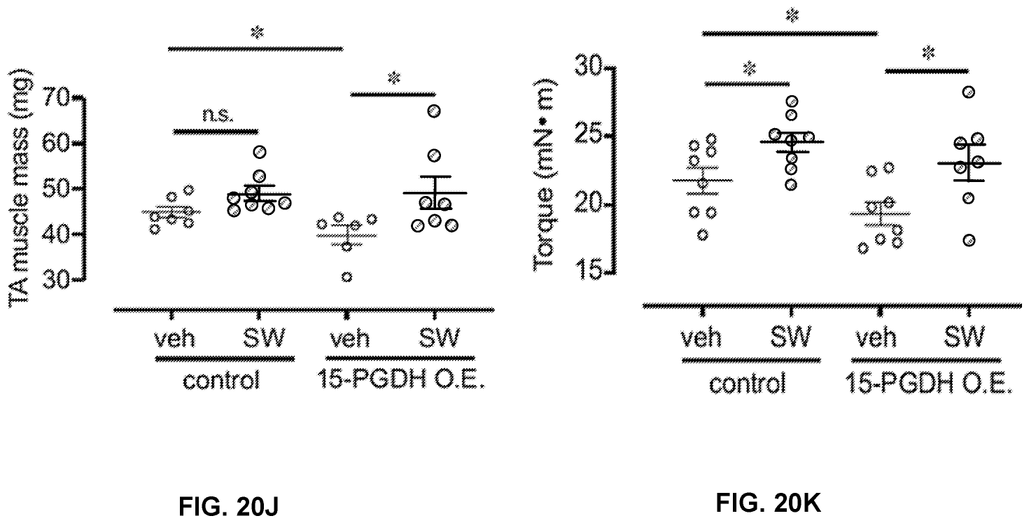

We reasoned that if 15-PGDH plays a major role in the loss of muscle function seen with aging, ectopic expression of the PGE2 degrading enzyme in muscles of young mice would have a deleterious effect on muscle function. To test this hypothesis, we used AAV9 to deliver and overexpress the 15-PGDH gene (Hpgd) under the control of the ubiquitous cytomegalovirus (CMV) promoter (FIG. 20A). We confirmed that upon intramuscular injection of AAV9-CMV-15-PGDH, expression of 15-PGDH was increased by qRT-PCR (FIG. 20B). Additionally, analysis by LC-MS/MS revealed a marked decrease in prostaglandins PGE2 and PGD2 in young muscles expressing 15-PGDH, similar to the decline in these prostaglandins seen in aged muscles (FIG. 20C). The reduction in these prostaglandins for a period of only one month resulted in a significant decrease in the average cross-sectional area of individual myofibers (FIGS. 20D and 20E) and an acute loss of muscle function, assayed as muscle mass and muscle force in young adult mice (FIGS. 20F and 20G). We analyzed markers of muscle atrophy by qRT-PCR and found that the atrogenes Trim63 (MuRFI) and Fbxo32 (Atrogin-1) and the autophagy genes p62, Lc3b, Atg4 and Atg6 were upregulated in muscles overexpressing 15-PGDH (FIG. 20H), in accordance with findings by others in acute models of atrophy (18-21). These data provide strong evidence that 15-PGDH overexpression plays a causal role in decreasing PGE2 and PGD2 levels in muscle, which in turn, leads to a decrease in muscle mass and strength. Moreover, they show that 15-PGDH activity has a profound effect on muscle homeostasis and induces an atrophy phenotype.

To determine the specificity of SW for its target 15-PGDH, we performed a rescue experiment in young mice overexpressing the enzyme following intramuscular AAV9-mediated gene delivery. We reasoned that inhibition of the over-expressed enzyme by SW should overcome the deleterious effects seen upon 15-PGDH overexpression. Accordingly, we treated control and 15-PGDH overexpressing young mice systemically with vehicle or SW (FIG. 20I). We found that treatment with SW increased the mass (FIG. 20J) and strength (FIG. 20K) of 15-PGDH overexpressing young muscles. These data demonstrate that 15-PGDH inhibition using the small molecule SW specifically targets 15-PGDH resulting in improved muscle function.

### Increase in Strength in Aged Mice Mediated by PGE2 but not PGD2

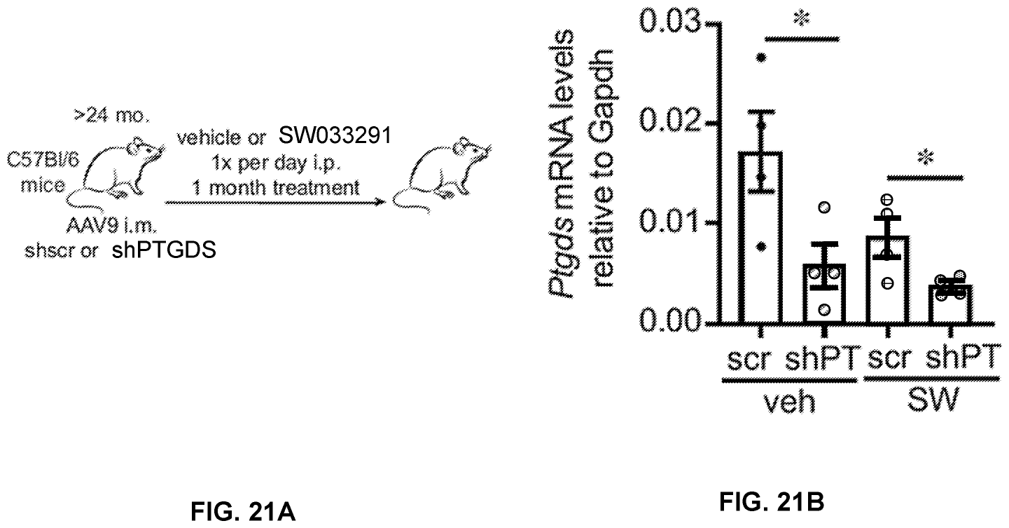

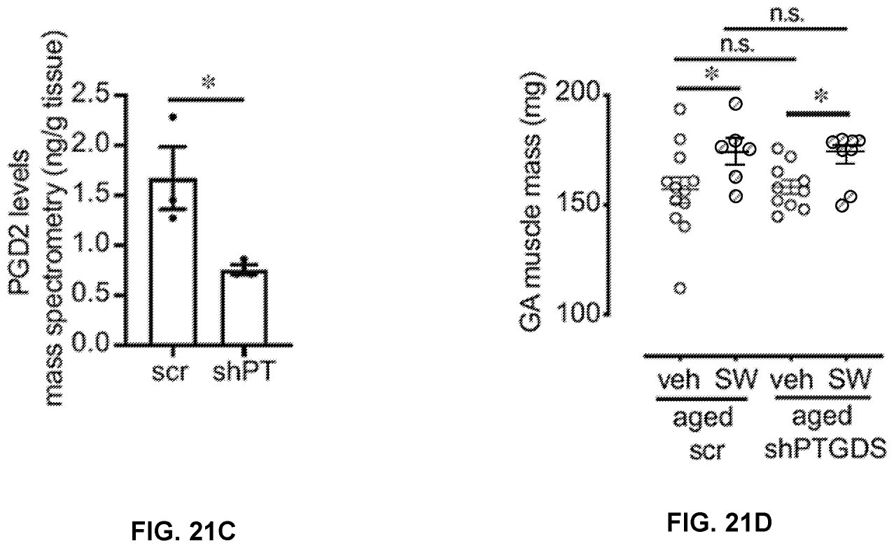

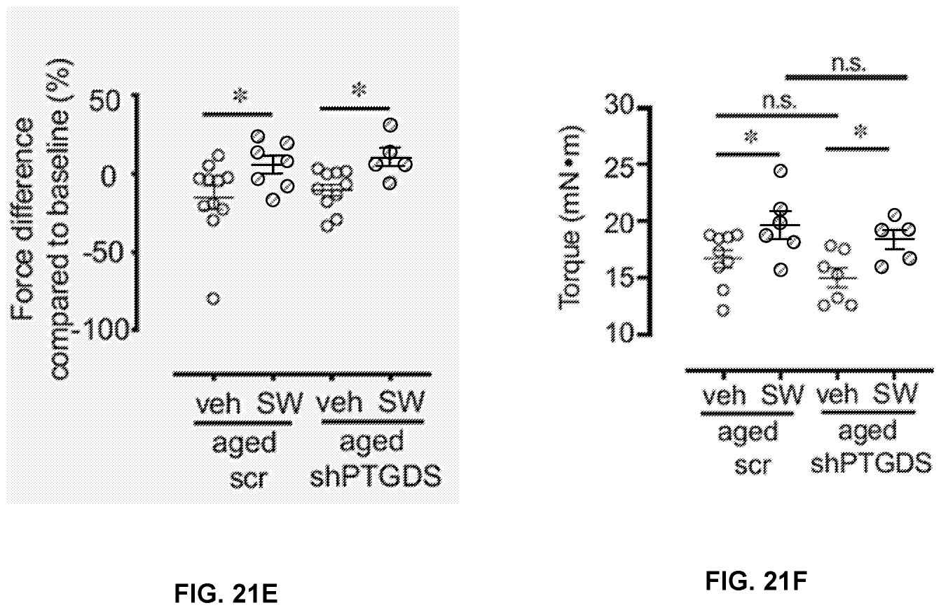

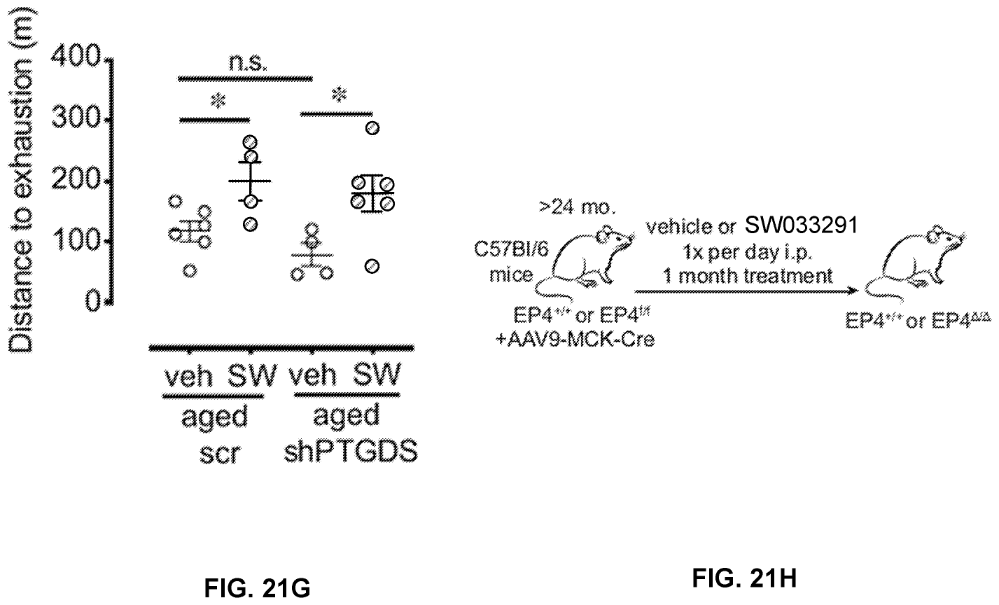

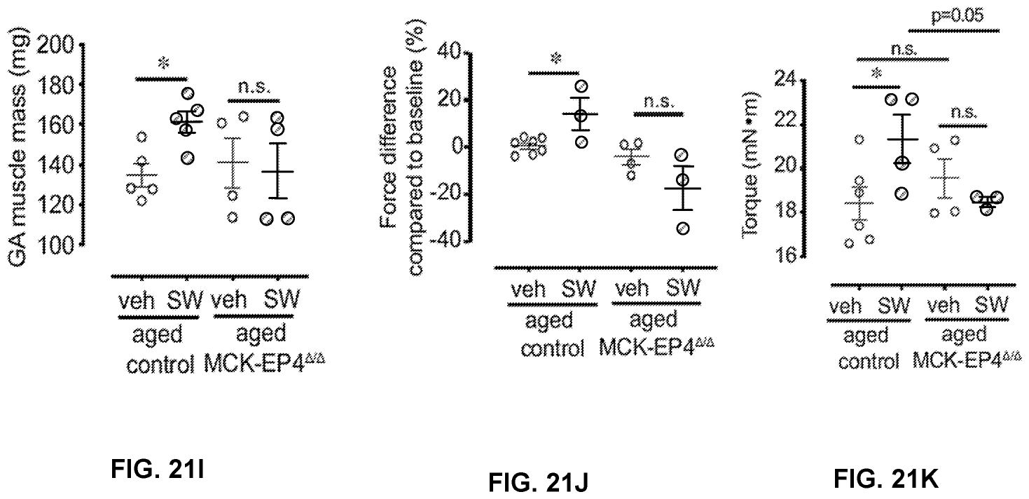

15-PGDH degrades both PGE2 and PGD2 in aged muscles. Notably, the two prostaglandins differ in their receptors and in their downstream signaling cascades (22). To determine which prostaglandin was responsible for driving the improvement in aged muscle function, we increased their levels by 15-PGDH inhibition using SW and inhibited the expression of the PGD2 synthesizing enzyme, PTGDS. This was achieved by intramuscular injection of aged muscles with an AAV9 virus encoding either an shRNA that targets PTGDS or a scrambled control shRNA and treating the mice for one month with the 15-PGDH inhibitor, SW or vehicle (FIG. 21A). We validated knockdown of PTGDS in transduced aged muscles by confirming reduced Ptgds mRNA levels by qRT-PCR and decreased levels of PGD2 by mass spectrometry (FIGS. 21B and 21C). Upon knockdown of PTGDS, an increase in muscle mass, force and endurance was seen after SW treatment (FIGS. 21D-G). These results suggest that PGE2, not PGD2, is the mediator of the increased muscle function seen in aged muscles upon 15-PGDH inhibition.

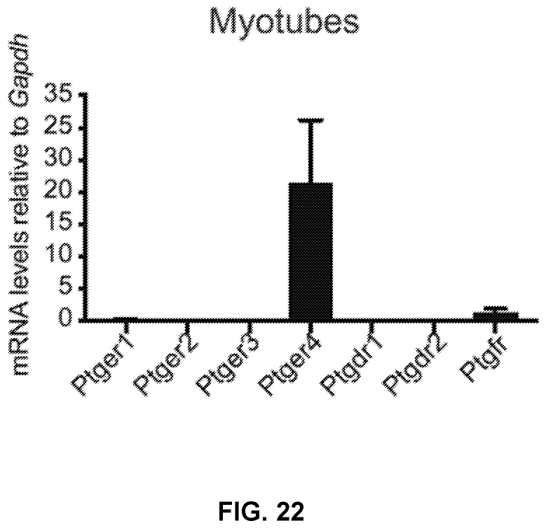

We performed additional experiments to substantiate the specific role of PGE2 in attenuating muscle atrophy in aged mice. Since three enzymes are responsible for PGE2 synthesis, cPGES, PGES1, and PGES2 (22), targeting the PGE2 synthesis pathway would entail a triple knockdown, which would be technically challenging. As an alternative approach, we focused on the PGE2 receptors in muscle. qRT-PCR revealed that the PGE2 receptor, EP4 (Ptger4), is the most highly expressed eicosanoid receptor in differentiated myotubes (FIG. 22A). To conclusively determine if the observed muscle hypertrophy was due to PGE2-mediated EP4 signaling in mature muscle myofibers in vivo, we created a mouse model in which the receptor was genetically ablated only in myofibers of GA muscles. This was achieved by intramuscular AAV9-mediated delivery of muscle creatine kinase (MCK)-promoter driven Cre to the GA myofibers of aged EP4f/f mice (MCK-EP4° ¹¹). Strikingly, loss of EP4 expression in myofibers of aged mice abrogated the beneficial effect on muscle mass and strength induced by SW-mediated 15-PGDH inhibition treatment for one month (FIGS. 21H-K). These data demonstrate that the observed effects of SW treatment are primarily mediated by PGE2 signaling through the EP4 receptor on aged myofibers.

### Increase in Mitochondrial Function and Biogenesis Following 15-PGDH Inhibition

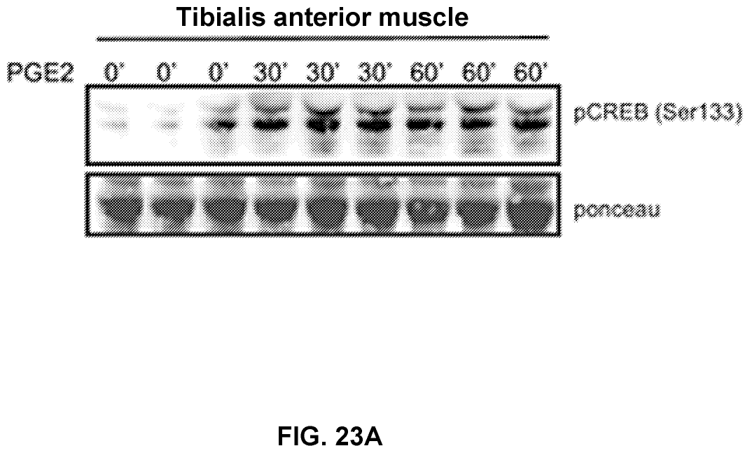

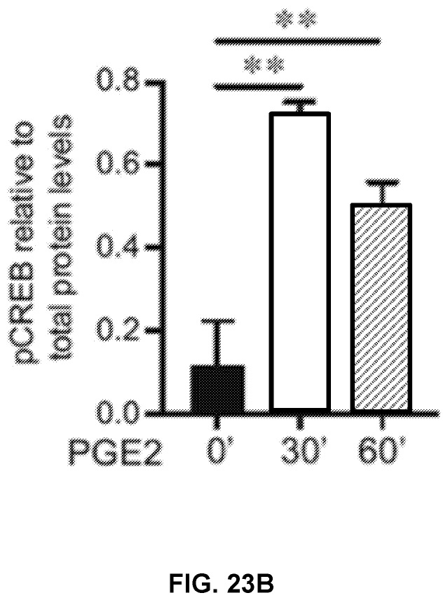

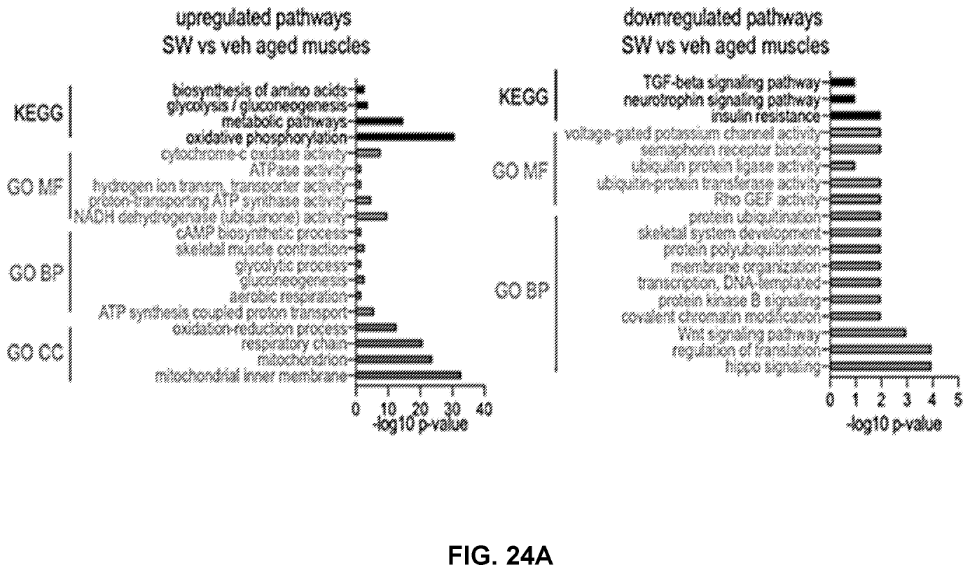

PGE2 signaling through the G-coupled protein receptor, EP4, is known to be mediated by cyclic AMP (cAMP) (12, 22, 23). We confirmed that PGE2 activates the cyclic AMP response element binding protein (CREB) in skeletal muscles (FIGS. 23A and 23B).

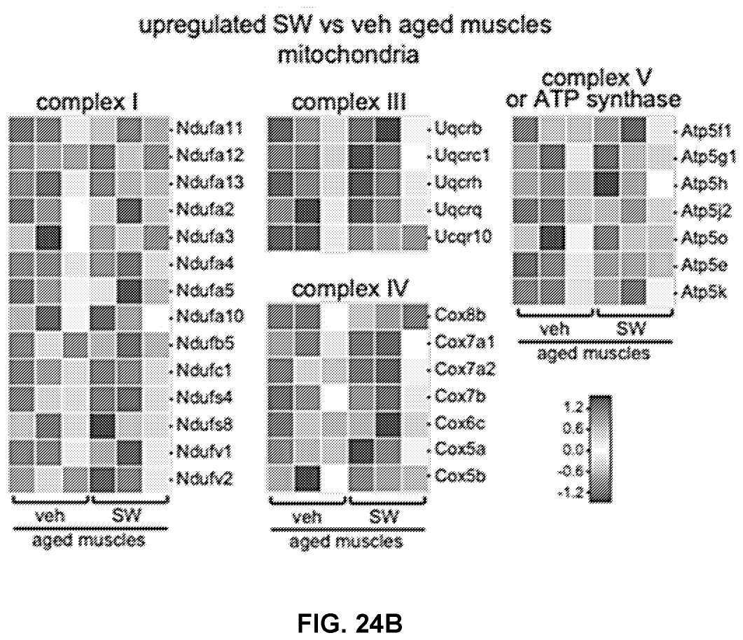

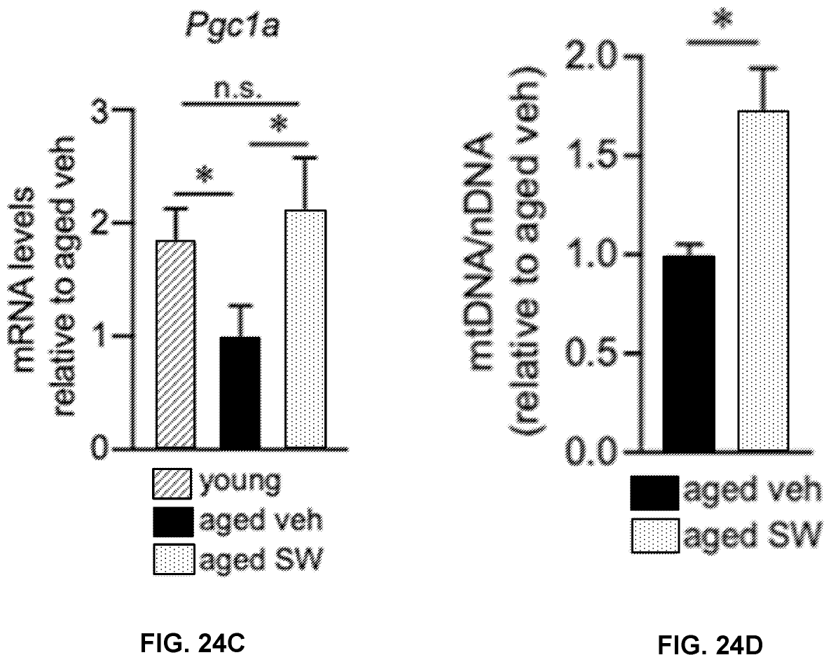

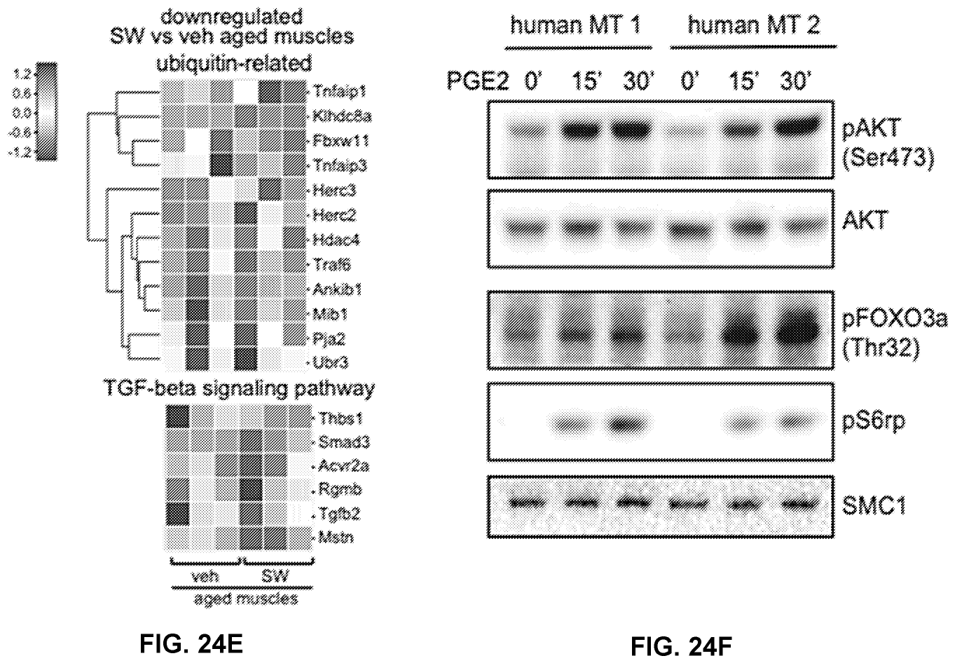

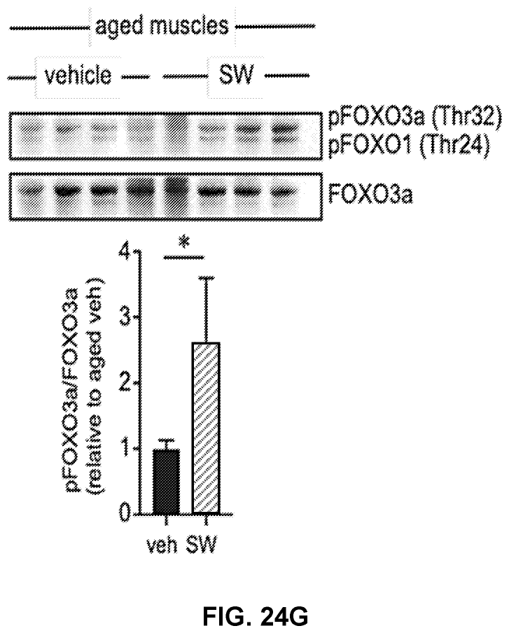

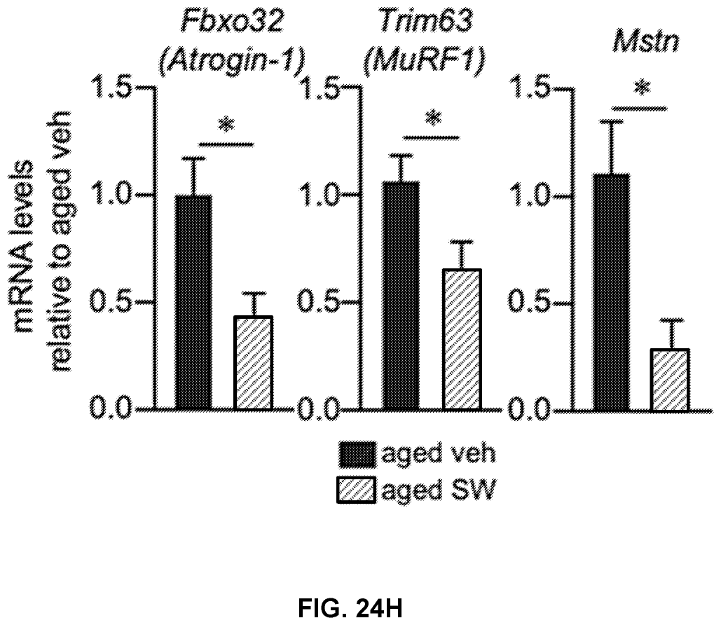

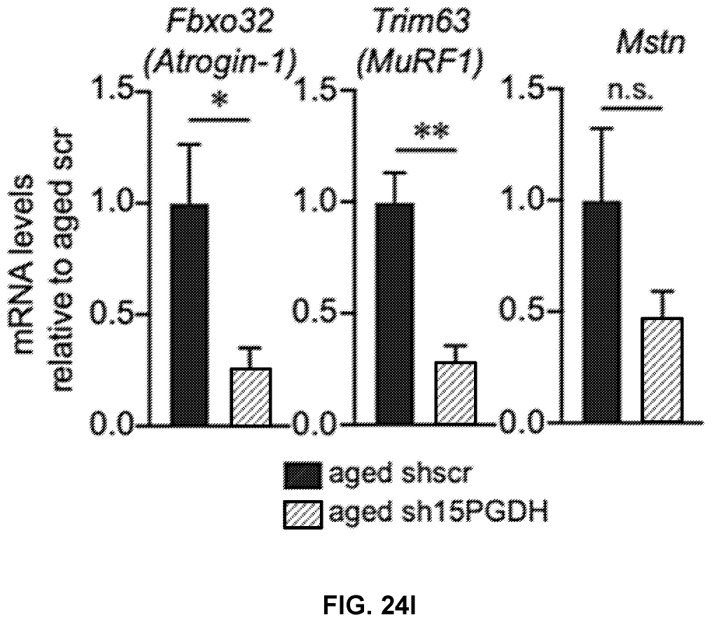

To identify downstream signaling pathways through which PGE2 exerts its effects on aged muscles, we performed an unbiased transcriptomic analysis of vehicle and SW-treated aged muscles. Most striking was the strong enrichment for mitochondrial pathways, including mitochondrial oxidative phosphorylation, ATP synthesis and other metabolic and energy producing processes (FIG. 24A). Numerous components of the mitochondria complexes I, II, IV and V of the electron transport chain were markedly increased in SW-treated aged muscles (FIG. 24B). When we assayed mRNA levels of a critical cofactor for mitochondrial biogenesis that has a CREB binding motif in its promoter, peroxisome proliferator-activated receptor gamma coactivator 1-alpha (Pgc1α) (24), we found that its level was restored to that seen in young muscles (FIG. 24C). Overall mitochondrial content was increased, as reflected by the increased ratio of mitochondrial to nuclear DNA following SW treatment of aged muscles (FIG. 24D). Together, these data provide strong evidence that PGE2 triggers a robust increase in mitochondrial number to meet the energetic requirements of muscle growth.

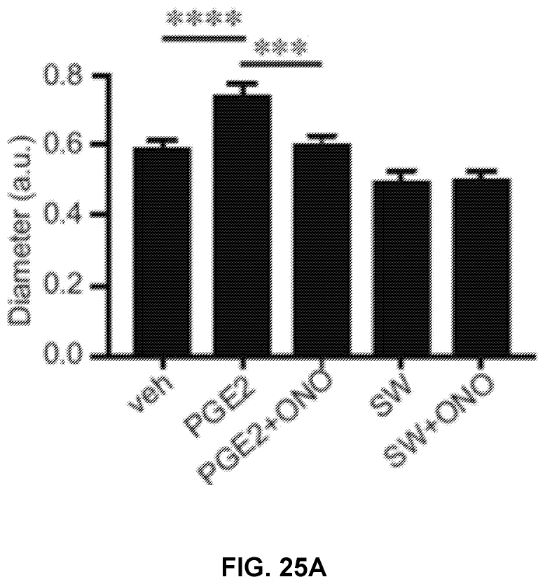

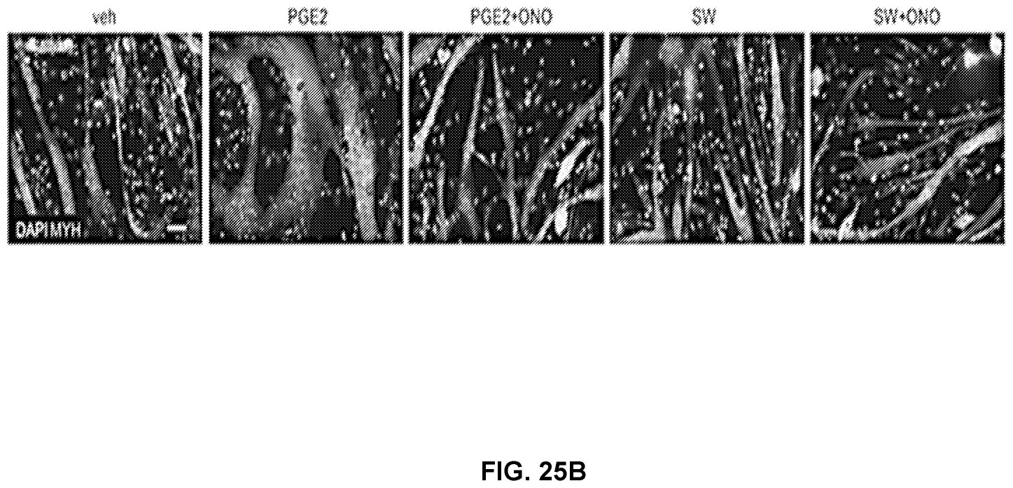

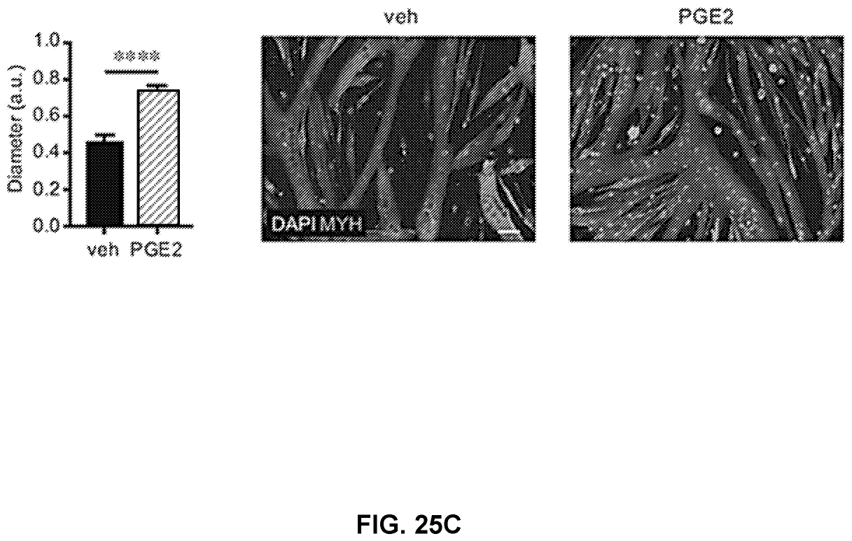

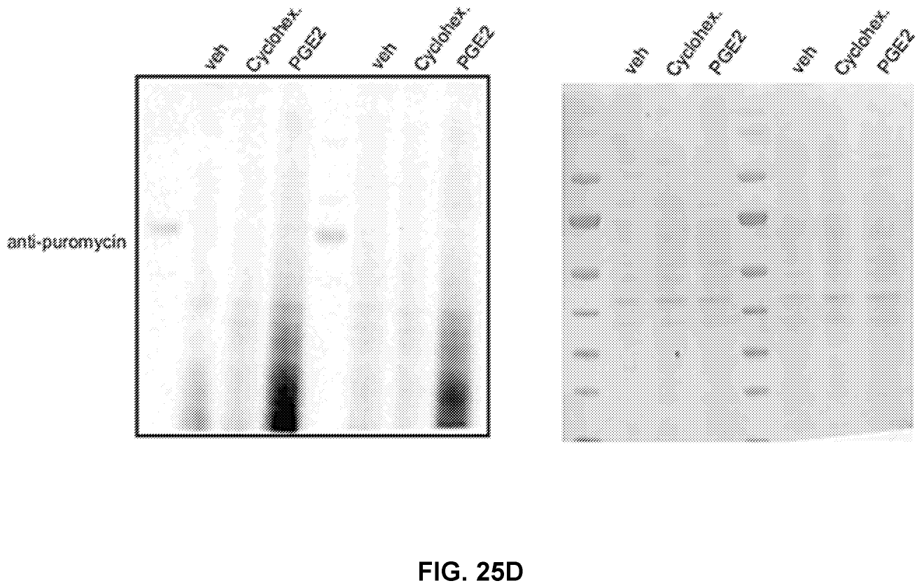

Gene expression analysis also revealed a decline in signaling pathways linked to age-related muscle atrophy. Among the top downregulated genes upon SW treatment of aged muscles were members of ubiquitin signaling pathways (FIGS. 24A and 24E). PGE2 signaling has previously been implicated in the activation of the AKT/FOXO pathway in non-muscle cells (12, 25, 26). We therefore sought to determine if this pathway might function in muscle to regulate the expression of E3 ubiquitin ligases that are known to play a role in muscle atrophy (27-29). To this end, muscle cells, in the absence of other cell types, were subjected to an acute exposure to PGE2. As shown by western blot analysis, differentiated myotubes derived from human donor muscle cells treated with PGE2 for 15 or 30 minutes exhibited increased levels of pAKT which inactivated FOXO (pFOXO3a) (FIG. 24F). Additionally, PGE2 treated myotubes activated the downstream target phospho-S6 ribosomal protein (pS6rp), indicative of increased protein synthesis (FIG. 24F) and exhibited a marked increase in diameter, not seen upon addition of the PGE2 antagonist (ONO-AE3-208) (FIGS. 25A-C). In corroboration, we observed an increase in protein synthesis quantified by puromycin incorporation after PGE2 treatment of myotubes (FIG. 25D). Treatment with SW had no effect on the diameter of cultured myotubes (FIGS. 25A and 25B), in accordance with its indirect mechanism of inhibiting 15-PGDH expression by resident interstitial cells in aged muscle tissue. These in vitro data show that PGE2 can act directly on myotubes to activate AKT signaling and enhance myotube growth and protein synthesis, providing evidence for a previously understudied role for PGE2 in countering muscle atrophy.

### Decreased Proteolysis and TGF-Beta Signaling Following 15-PGDH Inhibition in Aged Muscles

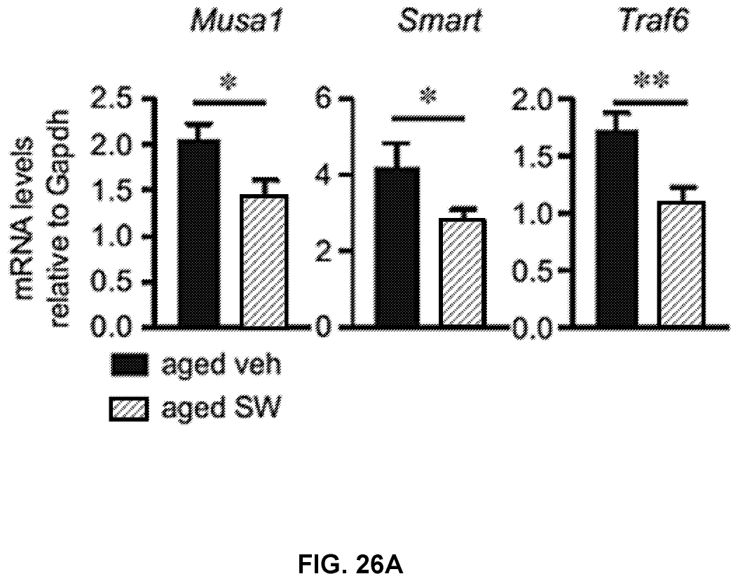

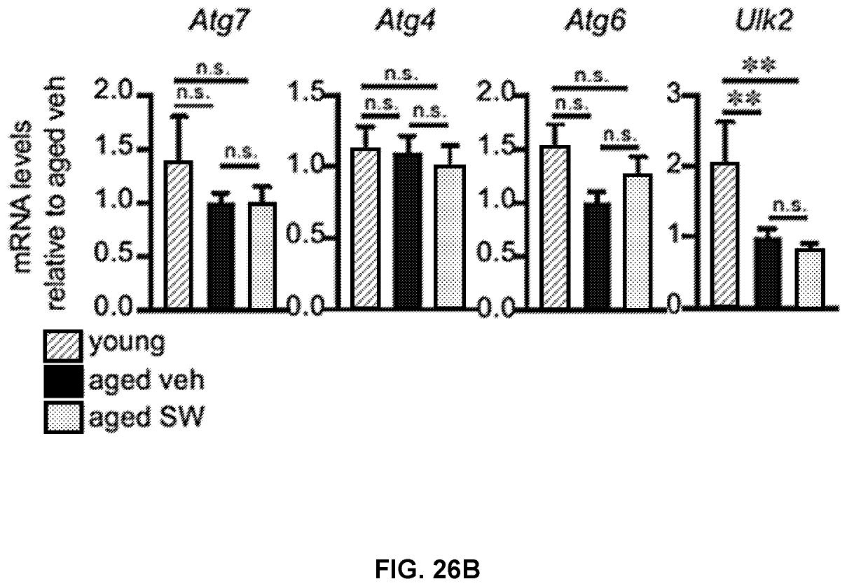

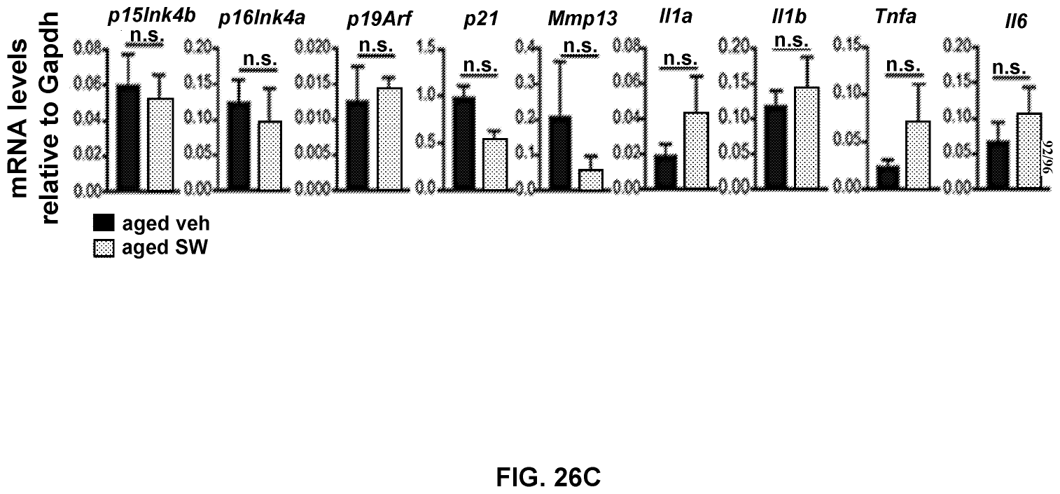

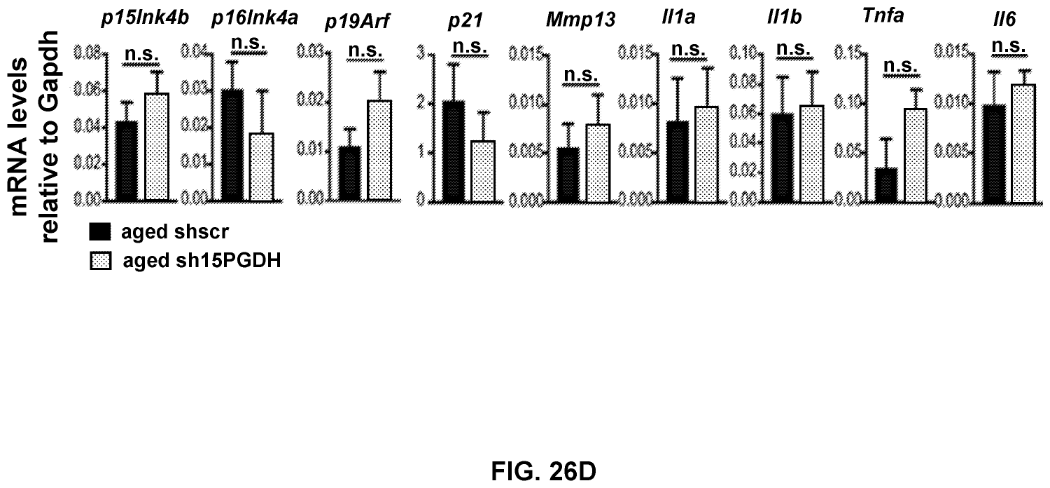

We sought to determine in vivo in aged muscle tissue if elevation of PGE2 due to 15-PGDH inhibition leads to signaling via the AKT/FOXO pathway, as seen in vitro in myotubes. We found that pFOXO was increased in SW treated aged muscles compared to vehicle treated controls (FIG. 24G). FOXO has previously been shown by others to play a role in decreasing expression of the muscle-specific atrophy-related E3 ubiquitin ligases Atrogin-1 (Fbxo32), MuRFI (Trim63), Musa] and Smart (30-32). Analysis by RT-qPCR revealed that expression of all of these atrogenes, as well as the E3 ubiquitin ligase Traf6 (33), was diminished in SW treated aged muscles compared to vehicle treated controls (FIGS. 24E, 24H, and 26A), suggesting that a modulation of proteolysis contributes to the attenuation of muscle atrophy. This finding fits well with our transcriptome analysis of aged muscles compared to young muscles which showed that the genes in the ubiquitin ligase pathway are among the top enriched upregulated genes in aged muscles (FIGS. 12A-D) and is in good agreement with the findings by others that atrogene expression is increased with aging (34-36). We observed a similar decrease in E3 ubiquitin ligase expression following a genetic inhibition of the 15-PGDH enzyme in aged muscles mediated by intramuscular delivery of an shRNA to 15-PGDH compared to scr shRNA control (FIG. 24I). Of interest, the histone deacetylase Hdac4, another mediator of muscle atrophy that deacetylates proteins such as MyHC and PGC1α leading to their ubiquitination as well as increasing expression of atrogenes Atrogin-1 and MuRFI (37, 38), was diminished in SW-treated muscles (FIG. 24E). These results show that PGE2 leads to a modulation of atrogene expression that tempers the increased protein degradation seen in aged muscles and contributes to the observed amelioration of muscle atrophy in aged muscles.

Our transcriptome analysis revealed a reduction in a second signaling pathway, the TGF-beta pathway, after one month of SW treatment, providing evidence of another synergistic beneficial effect of 15-PGDH inhibition on aged muscles. The expression of key TGF-beta pathway genes, such as myostatin, that are known to be detrimental to muscle function and associated with aged muscle atrophy in aging (Mstn, Tgfb2, Acrv2a, Smad3) (27), was decreased, which likely contributed to the observed attenuation of muscle atrophy (FIG. 24E). Notably, no significant changes were observed in other aging, inflammatory and autophagy markers assayed in muscles of SW treated aged mice (FIGS. 26B-D). Together, these results show that a one month 15-PGDH inhibition and consequent elevation of PGE2 in aged muscles stimulates several synergistic signaling pathways leading to the improvement in muscle function and attenuation of atrophy in aged mice.

### Discussion

Skeletal muscles make up 40% of the body's mass. After the age of 50, humans lose on average 15% of their muscle mass per decade (39) culminating in the drastic loss of muscle strength characteristic of sarcopenia. There are currently no therapies for sarcopenia and its healthcare burden is high (2). Here we discover that elevated expression of the prostaglandin degrading enzyme, 15-PGDH, is a new marker of aged muscles, both in mouse and humans. We find that increased 15-PGDH activity is not limited to muscle, but is a characteristic of many aged tissues, for example aged heart, skin, colon and spleen. The profound role of 15-PGDH in aging is highlighted by the finding that overexpression of this enzyme causes muscle wasting in young mice. In aged mice, inhibition of 15-PGDH, either by genetic knockdown or a small molecule, counters muscle atrophy and markedly increases muscle mass, strength and endurance. Using mass spectrometry and targeted loss of function experiments, we show that the amelioration of muscle function is due to increased PGE2 levels. We and others previously demonstrated the importance of PGE2 signaling in stimulating stem cells to regenerate damaged tissues in young mice (7-10). Here we demonstrate that PGE2 also acts on mature muscle myofibers and plays a crucial role in the maintenance of muscle tissue homeostasis. Importantly, our data suggest that 15-PGDH constitutes a therapeutic target to counter the debilitating muscle atrophy characteristic of sarcopenia.

To our knowledge, there are no prior reports that increased 15-PGDH activity leads to reduced PGE2 levels in aged tissues. Our study benefited from the LC-MS/MS method, which is capable of definitive resolution and quantification of highly similar prostaglandin family members in skeletal muscle. Accordingly, we were able to uncover the magnitude of PGE2 decline in aged muscle and implicate 15-PGDH in that decline. The significance of this enzyme in the atrophy phenotype is underscored by the finding that overexpression of the enzyme in young muscles leads to a striking loss of muscle mass and strength within one month. Taken together, our data highlight the causal role of 15-PGDH in decreasing muscle mass and function. Given that we detect increased 15-PGDH in a number of other aged tissues, this finding could have broad implications for age-related pathologies.

Our data suggest that an intercellular signaling mechanism plays a role in the reduction in PGE2 in aged muscles. Following either a senolytic treatment or genetic ablation of senescent cells in aged muscles, 15-PGDH levels are reduced and a concomitant increase in PGE2 is observed. These results implicate senescent interstitial cells in the aged muscle milieu as a major site of PGE2 catabolism. Of the senescent inflammatory cell types present in the aged muscle niche, macrophages appear to be a predominant cell type that expresses 15-PGDH and degrades PGE2. These cells appear to act indirectly via a paracrine mechanism to contribute to the muscle wasting phenotype, designated as “inflammaging” (43). This deleterious microenvironment can be overcome by eliminating senescent interstitial cells with senolytic treatments or by inhibiting 15-PGDH expression in aged muscles, both of which raise endogenous PGE2 levels sufficiently to attenuate muscle atrophy. Future studies are warranted to investigate this paracrine mechanism in detail. We postulate that similar tissue-resident senescent interstitial cells account for the elevated 15-PGDH we detected in other aged tissues.

Previous studies of the role of PGE2 in muscle protein homeostasis suggested PGE2 induces protein degradation, however, these studies were performed on denervated excised muscles undergoing rapid muscle protein catabolism precipitated by removal of the muscle from the body (44, 45). In contrast, here we provide evidence in live mice that inhibition of 15-PGDH impedes PGE2 degradation and leads to modulation of endogenous PGE2 levels within a physiological range that suffices to ameliorate muscle atrophy. Our data fit well with prior studies in which perturbation of COX enzyme levels revealed a role for prostaglandins in muscle hypertrophy and recovery from muscle atrophy (22, 46, 47). However, since COX2 is critical to the synthesis of prostaglandins with antagonistic effects, it is not an ideal therapeutic target. Here we reveal a previously unappreciated link between PGE2 signaling and muscle atrophy via multiple signaling pathways-TGF-beta, cAMP/CREB, AKT/FOXO and mitochondrial function—that synergize to augment muscle function and attenuate muscle atrophy.

Sarcopenia is a multifactorial disease, a compendium of dysregulated signaling pathways that culminate in chronic inflammation, muscle denervation, defective mitochondria, and disrupted proteostasis (4, 48, 49). In particular, mitochondrial function is impaired (50). To resolve the mechanisms underlying the beneficial effects of 15-PGDH inhibition on muscle function, we took an unbiased approach. A transcriptome analysis comparing aged muscles following a one-month treatment with a small molecule inhibitor of 15-PGDH with vehicle treated controls revealed that mitochondrial function is among the top upregulated pathways. PGE2 signaling through the EP4 receptor via cAMP/CREB could account for the observed increase in mitochondrial number and function, in agreement with prior reports (12, 22, 23). Similar to the beneficial effects on skeletal muscle previously shown for other cAMP inducing agents, such as B-adrenergic receptor (β-AR) agonists or corticotropin releasing factor receptor 2 (CRFR2) agonists, PGE2 induction of cAMP likely augments mitochondrial function by activating downstream transcriptional regulators with cAMP response elements (CREB binding motifs) that promote mitochondrial biogenesis, including the major mitochondrial regulator Pgc1a and other oxidative genes (51-53). This signaling cascade culminates in increased mitochondria mass and a marked improvement in muscle atrophy.

Our transcriptome analysis also revealed key signaling pathways that are downregulated after one month of 15-PGDH inhibition, including ubiquitin-proteasome pathway genes. In corroboration, this pathway was enriched in our transcriptome analysis of aged relative to young muscles. In agreement, others have reported elevated levels of the E3 ubiquitin ligases Atrogin-1 and MuRFI in aged rat (34, 35) and human muscles (36). Whether ubiquitin ligase expression plays a causal role in sarcopenia remains a matter of debate. Knockout models of certain E3 ubiquitin ligases, including Atrogin-1 and MuRFI, led to deleterious effects in muscle function (54, 55), but in the context of acute denervation atrophy had beneficial effects (27). Notably, these genetic models were not investigated in the context aging. Indeed, interventions such as rapalogs, sestrin, and Apelin that led to reduced atrogene expression (Atrogin-1 and MuRFI) in aged muscles (21, 56, 57) improved muscle mass and function and ameliorated sarcopenia. In agreement, we observed a decrease in expression of multiple E3-ubiquitin ligases upon 15-PGDH inhibition in aged muscles. Taken together, these data suggest that modulation of atrogene expression is beneficial to aged muscle function. In addition to atrogenes, we observed downregulation of Hdac4, which promotes atrophy by modulating E3 ubiquitin ligases (MuRFI and Atrogin-1), MyHC and Pgc1a levels (37, 38, 48) and of Traf6 which is an adapter protein and a nonconventional E3 ubiquitin ligase previously implicated in muscle atrophy (33). In addition to modulation of atrogene expression, an enhancement of autophagy has been implicated in the reversal of aging phenotypes downstream of AKT/FOXO signaling (21, 30), that was not apparent in our transcriptome analyses. Here we show that partial inhibition of 15-PGDH in aged mice leads to a reduction in a number of these atrophy markers resulting in improved muscle mass and function.

We also observed a striking downregulation of a second pathway, the TGF-beta signaling pathway in the transcriptome of SW treated aged muscles. A prominent member of this family, Myostatin, has marked suppressive effects on muscle growth, and its loss in knockout animals is associated with dramatic hypertrophy (58). Myostatin signals through activin receptors and downstream Smad transcription factors, turning off the AKT pathway and protein synthesis, while triggering the expression of ubiquitin ligases that orchestrate the degradation of muscle proteins (59). Several genes in the TGF-beta pathway, including myostatin, transforming growth factor beta-2 (TGFβ-2) and Activin receptor type-2A were markedly reduced in the transcriptome of SW-treated aged muscles.

Taken together, here we uncover 15-PGDH as a previously unrecognized marker and therapeutic target for strategies that aim to ameliorate the muscle wasting associated with aging and sarcopenia. Our intervention is advantageous, as it entails a physiological restoration of homeostatic levels of PGE2 in aged mice to those found in young mice. The resulting moderate increase in PGE2 levels modulates several signaling pathways to promote mitochondrial biogenesis and function while inhibiting TGF-beta and ubiquitin proteosome pathways, leading to an increase in muscle function. Since 15-PGDH activity is elevated in a range of tissues, we postulate that its partial inhibition could have beneficial effects that extend beyond skeletal muscle during aging.

### REFERENCES

1. S. Cohen, J. A. Nathan, A. L. Goldberg, Muscle wasting in disease: molecular mechanisms and promising therapies. Nat Rev Drug Discov 14, 58-74 (2015).
2. Y. Rolland et al., Sarcopenia: its assessment, etiology, pathogenesis, consequences and future perspectives. J Nutr Health Aging 12, 433-450 (2008).
3. R. A. Fielding et al., Sarcopenia: an undiagnosed condition in older adults. Current consensus definition: prevalence, etiology, and consequences. International working group on sarcopenia. J Am Med Dir Assoc 12, 249-256 (2011).
4. L. Larsson et al., Sarcopenia: Aging-Related Loss of Muscle Mass and Function. Physiol Rev 99, 427-511 (2019).
5. C. E. Lee, A. McArdle, R. D. Griffiths, The role of hormones, cytokines and heat shock proteins during age-related muscle loss. Clin Nutr 26, 524-534 (2007).
6. K. Lenk, G. Schuler, V. Adams, Skeletal muscle wasting in cachexia and sarcopenia: molecular pathophysiology and impact of exercise training. J Cachexia Sarcopenia Muscle 1, 9-21 (2010).
7. A. T. V. Ho et al., Prostaglandin E2 is essential for efficacious skeletal muscle stem-cell function, augmenting regeneration and strength. Proc Natl Acad Sci USA 114, 6675-6684 (2017).
8. H. Chen et al., Prostaglandin E2 mediates sensory nerve regulation of bone homeostasis. Nat Commun 10, 181 (2019).
9. T. E. North et al., Prostaglandin E2 regulates vertebrate haematopoietic stem cell homeostasis. Nature 447, 1007-1011 (2007).
10. Y. Zhang et al., TISSUE REGENERATION. Inhibition of the prostaglandin-degrading enzyme 15-PGDH potentiates tissue regeneration. Science 348, aaa2340 (2015).
11. J. K. Prasain, H. D. Hoang, J. W. Edmonds, M. A. Miller, Prostaglandin extraction and analysis in Caenorhabditis elegans. J Vis Exp, (2013).
12. D. Wang, R. N. Dubois, Eicosanoids and cancer. Nat Rev Cancer 10, 181-193 (2010).
13. Y. H. Wu et al., Structural basis for catalytic and inhibitory mechanisms of human prostaglandin reductase PTGR2. Structure 16, 1714-1723 (2008).
14. U. Raue et al., Transcriptome signature of resistance exercise adaptations: mixed muscle and fiber type specific profiles in young and old adults. J Appl Physiol (1985) 112, 1625-1636 (2012).
15. B. G. Childs, M. Dunk, D. J. Baker, J. M. van Deursen, Cellular senescence in aging and age-related disease: from mechanisms to therapy. Nat Med 21, 1424-1435 (2015).
16. D. J. Baker et al., Naturally occurring p16(Ink4a)-positive cells shorten healthy lifespan. Nature 530, 184-189 (2016).
17. J. Chang et al., Clearance of senescent cells by ABT263 rejuvenates aged hematopoietic stem cells in mice. Nature Medicine 22, 78-83 (2016).
18. L. Brocca et al., FoxO-dependent atrogenes vary among catabolic conditions and play a key role in muscle atrophy induced by hindlimb suspension. J Physiol 595, 1143-1158 (2017).
19. J. M. Sacheck et al., Rapid disuse and denervation atrophy involve transcriptional changes similar to those of muscle wasting during systemic diseases. FASEB J 21, 140-155 (2007).
20. M. F. O'Leary, A. Vainshtein, S. Iqbal, O. Ostojic, D. A. Hood, Adaptive plasticity of autophagic proteins to denervation in aging skeletal muscle. Am J Physiol Cell Physiol 304, C422-430 (2013).
21. J. Segales et al., Sestrin prevents atrophy of disused and aging muscles by integrating anabolic and catabolic signals. Nat Commun 11, 189 (2020).
22. M. Korotkova, I. E. Lundberg, The skeletal muscle arachidonic acid cascade in health and inflammatory disease. Nat Rev Rheumatol 10, 295-303 (2014).
23. E. Ricciotti, G. A. FitzGerald, Prostaglandins and inflammation. Arterioscler Thromb Vasc Biol 31, 986-1000 (2011).
24. P. J. Femandez-Marcos, J. Auwerx, Regulation of PGC-lalpha, a nodal regulator of mitochondrial biogenesis. Am J Clin Nutr 93, 8845-890 (2011).
25. H. Fujino, J. W. Regan, EP(4) prostanoid receptor coupling to a pertussis toxin-sensitive inhibitory G protein. Mol Pharmacol 69, 5-10 (2006).
26. V. Konya, G. Marsche, R. Schuligoi, A. Heinemann, E-type prostanoid receptor 4 (EP4) in disease and therapy. Pharmacol Ther 138, 485-502 (2013).
27. P. Bonaldo, M. Sandri, Cellular and molecular mechanisms of muscle atrophy. Dis Model Mech 6, 25-39 (2013).
28. M. D. Gomes, S. H. Lecker, R. T. Jagoe, A. Navon, A. L. Goldberg, Atrogin-1, a muscle-specific F-box protein highly expressed during muscle atrophy. Proc Natl Acad Sci U.S. 98, 14440-14445 (2001).
29. S. C. Bodine et al., Akt/mTOR pathway is a crucial regulator of skeletal muscle hypertrophy and can prevent muscle atrophy in vivo. Nat Cell Biol 3, 1014-1019 (2001).
30. G. Milan et al., Regulation of autophagy and the ubiquitin-proteasome system by the FoxO transcriptional network during muscle atrophy. Nat Commun 6, 6670 (2015).
31. M. Sandri et al., Foxo transcription factors induce the atrophy-related ubiquitin ligase atrogin-1 and cause skeletal muscle atrophy. Cell 117, 399-412 (2004).
32. T. N. Stitt et al., The IGF-1/PI3K/Akt pathway prevents expression of muscle atrophy-induced ubiquitin ligases by inhibiting FOXO transcription factors. Mol Cell 14, 395-403 (2004).
33. P. K. Paul et al., Targeted ablation of TRAF6 inhibits skeletal muscle wasting in mice. J Cell Biol 191, 1395-1411 (2010).
34. M. Altun et al., Muscle wasting in aged, sarcopenic rats is associated with enhanced activity of the ubiquitin proteasome pathway. J Biol Chem 285, 39597-39608 (2010).
35. S. Clavel et al., Atrophy-related ubiquitin ligases, atrogin-1 and MuRFI are up-regulated in aged rat Tibialis Anterior muscle. Mech Ageing Dev 127, 794-801 (2006).
36. S. Welle, A. I. Brooks, J. M. Delehanty, N. Needler, C. A. Thornton, Gene expression profile of aging in human muscle. Physiol Genomics 14, 149-159 (2003).
37. L. Luo et al., HDAC4 Controls Muscle Homeostasis through Deacetylation of Myosin Heavy Chain, PGC-lalpha, and Hsc70. Cell Rep 29, 749-763 e712 (2019).
38. V. Moresi et al., Myogenin and class II HDACs control neurogenic muscle atrophy by inducing E3 ubiquitin ligases. Cell 143, 35-45 (2010).
39. S. von Haehling, J. E. Morley, S. D. Anker, An overview of sarcopenia: facts and numbers on prevalence and clinical impact. J Cachexia Sarcopenia Muscle 1, 129-133 (2010).
40. M. Combrinck et al., Levels of CSF prostaglandin E2, cognitive decline, and survival in Alzheimer's disease. J Neurol Neurosurg Psychiatry 77, 85-88 (2006).
41. B. Cryer, J. S. Redfem, M. Goldschmiedt, E. Lee, M. Feldman, Effect of aging on gastric and duodenal mucosal prostaglandin concentrations in humans. Gastroenterology 102, 1118-1123 (1992).
42. M. K. Young, R. M. Bocek, P. T. Herrington, C. H. Beatty, Aging: effects on the prostaglandin production by skeletal muscle of male rhesus monkeys (Macaca mulatta). Mech Ageing Dev 16, 345-353 (1981).

43. C. Franceschi, P. Garagnani, P. Parini, C. Giuliani, A. Santoro, Inflammaging: a new immune-metabolic viewpoint for age-related diseases. Nat Rev Endocrinol 14, 576-590 (2018).
44. H. P. Rodemann, A. L. Goldberg, Arachidonic acid, prostaglandin E2 and F2 alpha influence rates of protein turnover in skeletal and cardiac muscle. J Biol Chem 257, 1632-1638 (1982).
45. H. P. Rodemann, L. Waxman, A. L. Goldberg, The stimulation of protein degradation in muscle by Ca2+ is mediated by prostaglandin E2 and does not require the calcium-activated protease. J Biol Chem 257, 8716-8723 (1982).
46. B. A. Bondesen, S. T. Mills, G. K. Pavlath, The COX-2 pathway regulates growth of atrophied muscle via multiple mechanisms. Am J Physiol Cell Physiol 290, C1651-1659 (2006).
47. T. A. Trappe, S. Z. Liu, Effects of prostaglandins and COX-inhibiting drugs on skeletal muscle adaptations to exercise. J Appl Physiol (1985) 115, 909-919 (2013).
48. C. Ibebunjo et al., Genomic and proteomic profiling reveals reduced mitochondrial function and disruption of the neuromuscular junction driving rat sarcopenia. Mol Cell Biol 33, 194-212 (2013).
49. C. Ubaida-Mohien et al., Discovery proteomics in aging human skeletal muscle finds change in spliceosome, immunity, proteostasis and mitochondria. Elife 8, (2019).
50. R. Calvani et al., Mitochondrial pathways in sarcopenia of aging and disuse muscle atrophy. Biol Chem 394, 393-414 (2013).
51. R. Berdeaux, R. Stewart, cAMP signaling in skeletal muscle adaptation: hypertrophy, metabolism, and regeneration. Am J Physiol Endocrinol Metab 303, E1-17 (2012).
52. S. Herzig et al., CREB regulates hepatic gluconeogenesis through the coactivator PGC-1. Nature 413, 179-183 (2001).
53. S. Austin, J. St-Pierre, PGClalpha and mitochondrial metabolism-emerging concepts and relevance in ageing and neurodegenerative disorders. J Cell Sci 125, 4963-4971 (2012).
54. M. Sandri et al., Signalling pathways regulating muscle mass in ageing skeletal muscle: the role of the IGF1-Akt-mTOR-FoxO pathway. Biogerontology 14, 303-323 (2013).
55. T. Zaglia et al., Atrogin-1 deficiency promotes cardiomyopathy and premature death via impaired autophagy. J Clin Invest 124, 2410-2424 (2014).
56. G. A. Joseph et al., Partial Inhibition of mTORCl in Aged Rats Counteracts the Decline in Muscle Mass and Reverses Molecular Signaling Associated with Sarcopenia. Mol Cell Biol 39, (2019).

57. C. Vinel et al., The exerkine apelin reverses age-associated sarcopenia. Nat Med 24, 1360-1371 (2018).
58. A. C. McPherron, A. M. Lawler, S. J. Lee, Regulation of skeletal muscle mass in mice by a new TGF-beta superfamily member. Nature 387, 83-90 (1997).
59. Y. Elkina, S. von Haehling, S. D. Anker, J. Springer, The role of myostatin in muscle wasting: an overview. J Cachexia Sarcopenia Muscle 2, 143-151 (2011).

### Materials and Methods.

### Mice

We performed all experiments and protocols in compliance with the institutional guidelines of Stanford University and Administrative Panel on Laboratory Animal Care (APLAC). Middle aged (18-20 mo.) and aged (>24 mo.) mice C57BL/6 were obtained the US National Institute on Aging (NIA) for aged muscle studies, and young (2-4 mo.) wild-type C57BL/6 mice from Jackson Laboratory. INK-ATTAC mice were generated as previously described (1). INK-ATTAC healthspan assessments. INK-ATTAC mice were crossed onto the C57BL6/J genetic background and maintained in specific-pathogen free housing on a 12-hour dark/light cycle for study duration. At 12 months of age, male mice were either utilized for baseline healthspan assessments and terminal muscle harvest or randomized to receive twice-weekly vehicle or AP20187 (2 mg/kg intraperitoneal injection; B/B homodimerizer, Clontech) until assessment and sacrifice at 28 months of age (2). Mice were treated for 1 month once a day by intraperitoneal injection with 5 mg/kg of SW033291 (SW) (Cayman Chemicals) or vehicle (10% ethanol, 5% Cremophor EL, 85% D5W (Dextrose 5% Water)) as previously described (3). Time and distance to exhaustion was performed as previously described (4) for SW-treated mice and their controls.

For ABT-263 treatments, 20 month old C57/B16 mice were treated with vehicle (ethanol:polyethylene glycol 400:Phosal 50 PG) or 50 mg/kg/day ABT-263 (in ethanol:polyethylene glycol 400:Phosal 50 PG) by oral gavage for 2 cycles of 1 week with a 2 week rest period between cycles as described previously (5). Intramuscular injection of PGE2 was carried out in young mice with either 13 nmol PGE2 (Cayman Chemicals) or vehicle control (PBS) into the TA muscle. Mouse transgenic strains were purchased from The Jackson Laboratory (EP4^{flox/flox}; EP4O) No. 028102 (6). We validated these genotypes by appropriate PCR-based strategies. Studies were performed with male mice.

### Primary Cell Isolation Using FACs

We isolated and enriched myogenic cells as previously described (6-9). Briefly, hindlimb muscles were minced and digested using a collagenase and dispase solution by the MACs Dissociator (Miltenyi). Using FACs, for myogenic stem and progenitor cells we isolated cells negative for the hematopoietic lineage and non-muscle cells (CD45-/CD11b-/CD31-/Scal-) and sorted for a7-integrin*cells markers. For macrophages isolation we sorted a7-/Cd11b+/Cd11c-/F4/80⁺ populations. For endothelial cells we sorted a7-/Cd11b-/Cd11c/CD31+. We generated and analyzed flow cytometry scatter plots using FlowJo v10.0.

Intramuscular AAV9 Delivery of shRNA and MCK-Cre

shRNA directed against Hpgd (15-PGDH) (NM_008278) was integrated into AAV9 under U6 promoter dependency and with eGFP (AAV9-eGFP-U6-sh15PGDH) (Vector Biolabs). Control mice were treated with a similar construction containing a scramble peptide sequence instead of sh15PGDH (AAV9-eGFP-U6-shscr). Cre was integrated into AAV9 under the muscle specific tMCK promoter and with eGFP (AAV9-tMCK-eGFP-WPRE) (Vector Biolabs). Overexpression of Hpgd (15-PGDH) was achieved by integrating AAV9 under CMV promoter and with eGFP under IRES (AA9-CMV-m-HPGD-IRES-eGFP) (Vector Biolabs). The control virus was AAV9-tMCK-eGFP-WPRE. Knockdown of Ptdgs was achieved using AAV9 integrated under U6 promoter dependency (AAV9-GFP-U6-m-PTGDS-shRNA) and control mice were injected with scrambled (AAV9-GFP-U6-scrmb-shRNA) (Vector Biolabs) at a final concentration of 2×10¹¹ GC/GA. 3-4 or >24 month old C57B1/6 mice were subject to two intramuscular injections into the Gastrocnemius (GA) with 20p dilution of the above described AAV9 particles in PBS to a final concentration of 2×10¹¹ particles/GA and/or one intramuscular injection into the Tibialis anterior (TA) to a final concentration of 2×10¹¹ GC/TA.

### Immunofluorescence Staining and Imaging

We collected and prepared recipient Tibialis anterior (TA) or gastrocnemius (GA) muscle tissues for histology as previously described (6). We fixed transverse sections or isolated myofibers from muscles using 4% PFA, blocked and permeabilized using PBS/1% BSA/0.1% Triton X-100 and incubated with biotin-anti-CD11b (BD Biosciences, catalog #553309, 1:100), anti-15-PGDH (Novus Biologicals, catalog # NB200-179SS, 1:100), anti-LAMININ (Millipore, clone A5, catalog #05-206, 1:200) and then with AlexaFluor secondary Antibodies (Jackson ImmunoResearch Laboratories, 1:200), Streptavidin-Cy3 (Biolegend, 1:500) or wheat germ agglutinin-Alexa 647 conjugate (WGA, Thermo Fisher Scientific). We counterstained nuclei with DAPI (Invitrogen).

Fiber typing was performed by immunohistochenistry of frozen 10 uM cut sections and mounted on glass slides. Air dried sections were immediately blocked in PBS/1% goat serum for 1 hour at room temperature and immunohistostained using antibodies to MHC2a (SC71 from DSHB, 1:1000), MHC2b (BF-F3 from DSHB, 1:100) (10, 11), and laminin (Millipore, clone A5, catalog #05-206, 1:200) diluted in PBS/1% goat serum overnight at 4° C. Secondaries antibodies against IgGIAlexa488, IgM Alexa 405, and IgG2b Alexa647 (Jackson Immu-noReserch Laboratories, 1:500) diluted in PBS/1% BSA were applied for 1 hour at room temperature and then nuclei were counterstained with DAPI (Invitrogen).

Images were acquired using KEYENCE BZ-X700 all in one fluorescence microscope with 20x/0.75 NA. objectives and individual fields were stitched and analyzed using Keyence Advanced Analysis Software.

For cultured myotubes we performed fixation using 4% PFA, blocking and permeabilization using PBS/1% BSA/0.1% Triton X-100 and staining with primary antibodies anti-MYH (Thermo Fisher Scientific, catalog #14-6503-82, clone MF-20, 1:500) and then with AlexaFluor secondary Antibodies (Jackson ImmunoResearch Laboratories, 1:500). We counterstained nuclei with DAPI (Invitrogen). We acquired images using the KEYENCE BZ-X700 all-in-one fluorescence microscope (Keyence) with 20x/0.75 N.A. objectives. We analyzed the fiber area using the Keyence Advanced Analysis Software. For fiber area the entire maximum cross-sectional area of the muscle was quantified or at least 10 fields of LAMININ or WGA stained myofiber cross-sections encompassing over 400 myofibers were captured for each mouse as above. For fiber typing analysis, the MATLAB application SMASH-Semi-Automatic Muscle Analysis Using Segmentation of Histology, was used as previously described (12). Data analyses were blinded. The researchers performing the imaging acquisition and scoring were unaware of treatment condition given to sample groups analyzed.

### Cell Culture

Primary murine myoblasts were grown in myogenic cell culture medium containing DMEM/F10 (50:50), 15% FBS, 2.5 ng ml⁻¹ fibroblast growth factor-2 and 1% penicillin-streptomycin. Primary human progenitors from Pectoralis muscle from two 59 year-old females as previously described (13) and grown using SkGM-2 Skeletal Muscle Growth Medium (Lonza, CC-3245). For differentiation experiments, confluent myoblasts were grown in medium containing 5% horse serum, DMEM. We added 10 ng/ml Prostaglandin E2 (Cayman Chemicals), 1 μM of SW033291 (ApexBio) or 1 μM of ONO-AE3-208 (Cayman Chemicals) to day 4 differentiated murine myotubes or day 7 differentiated human myotubes.

### Protein Synthesis by In Vitro SUnSET.

The SUnSET assay was used to monitor the rate of protein synthesis as previously described (4). Briefly, 10 min prior harvesting the cells, puromycin was added to culture medium at 1 pg/ml. As a control, cycloheximide to block protein translation was added. Cell extracts were then processed for western blotting using anti-puromycin 12D10 antibody (Millipore).

### Quantitative RT-PCR

We isolated RNA from MuSCs, myoblasts and myotubes using the RNeasy Kit (Qiagen). Muscle samples were snap frozen in liquid nitrogen, then homogenized in Trizol (Invitrogen) using the FastPrep FP120 homogenizer (MP Biomedicals) before isolating RNA. We reverse-transcribed cDNA from total mRNA from each sample using the SensiFAST™ cDNA Synthesis Kit (Bioline). We subjected cDNA to RT-PCR using SYBR Green PCR Master Mix (Applied Biosystems) or TaqMan Assays (Applied Biosystems) in an ABI 7900HT Real-Time PCR System (Applied Biosystems). We cycled samples at 95° C. for 10 min and then 40 cycles at 95° C. for 15 s and 60° C. for 1 min. To quantify relative transcript levels, we used 2-ΔΔCt to compare treated and untreated samples and expressed the results relative to Gapdh.

For SYBR Green qRT-PCR, we used the following primer sequences:

Gapdh,
forward
5′-TTCACCACCATGGAGAAGGC-3′,
reverse
5′-CCCTTTTGGCTCCACCCT-3′;
Hpgd,
forward
5′-TCCAGTGTGATGTGGCTGAC-3′,
reverse
5′-ATTGTTCACGCCTGCATTGT-3′;
Ptger1,
forward
5′ GTGGTGTCGTGCATCTGCT-3′,
reverse
5′-CCGCTGCAGGGAGTTAGAGT-3′;
Ptger2,
forward
5′-ACCTTCGCCATATGCTCCTT-3′,
reverse
5′-GGACCGGTGGCCTAAGTATG-3′;
Fbxo32 (Atrogin1),
forward
5′-TAGTAAGGCTGTTGGAGCTGATAG-3′,
reverse
5′-CTGCACCAGTGTGCATAAGG-3′;
Trim63
forward
5′-CATCTTCCAGGCTGCGAATC-3′,
reverse
5′-ACTGGAGCACTCCTGCTTGT-3′;
Atg4,
forward
5′-ATGGAGTCAGTTATGTCCAA-3′,
reverse
5′-CAATCGGGGAAAACTTCCTT-3′;
Atg6
forward
5′-GGAACTCACAGCTCCATTACTTA-3′,
reverse
5′-CATCCTGGCGAGTTTCAATAA-3′;
Pgc1a,
forward
5′-AGACAAATGTGCTTCGAAAAAGAA-3′,
reverse
5′-GAAGAGATAAAGTTGTTGGTTTGGC-3′;
Ptgdr1
forward
5′-CCCAGTCAGGCTCAGACTACA-3′,
reverse
5′-AAGTTTAAAGGCTCCATAGTACGC-3′;
Ptgdr2
forward
5′-AGCACACCCGATCAGTCAC-3′,
reverse-
5′-GTCACCCAGGAACCAGAAGA-3′;
Ptgfr
forward
5′-TCATGAAGGCCTACCAGAGATT-3′,
reverse
5′-CTGTGATCACCAGGCCACTA-3′
Musa1
forward
5′-CTTCAGTCTCGTGGAATGGTAATCTT-3′,
reverse
5′-TGCAGTACTGAATCGCCATAC-3′
Smart
forward
5′-TTTTTGAGGATGAGCTGGTGTGT-3′,
reverse
5′-AGGAACGCCTTGAGGTTATTGAG-3′
Traf6
forward
5′-TGCAAAAGATGGAACTGAGACATC-3′,
reverse
5′-TGGGACAATCCTCAATAATGTGTG-3′
Atf7
forward
5′-TCTGGGAAGCCATAAAGTCAGG-3′,
reverse
5′-GCGAAGGTCAGGAGCAGAA-3′
Bnip3
forward 5′-TGACAGCCCACCTCGC-3′,
reverse
5′-TCGACTTGACCAATCCCATA-3′
Ulk2
forward
5′-GCACCGCCAGAAAACTGAT-3′,
reverse
5′-GTTGGGCAATTCCTGAACAT-3′

For murine senescence markers and senescence associated markers we used the previously described primers (2). TaqMan Assays (Applied Biosystems) were used to quantify p21, Mstn, Ptger3 and Ptger4 in samples according to the manufacturer instructions with the TaqMan Universal PCR Master Mix reagent kit (Applied Biosystems). Transcript levels were expressed relative to Gapdh levels. For SYBR Green qPCR, Gapdh qPCR was used to normalize input cDNA samples. For Taqman qPCR, multiplex qPCR enabled target signals (FAM) to be normalized individually by their internal Gapdh signals (VIC). Mitochondrial copy number was quantified by using method and primers described previously (14).

### Microarray Data

The microarray gene expression profile was collected from the publicly available repository Gene Expression Omnibus (ncbi.nlm.nih.gov/geo). We analyzed microarray data from GSE25941 (15) for Hpgd expression.

### RNA-Seq

For RNA-seq, RNA was isolated from muscle lysates using Trizol reagent (Thermoscientific) and purified using Qiagen RNAEasy kit from. Libraries were constructed from RNA with the TruSEQ RNA Library Preparation Kit v2 (Illumina) and sequenced to 30-40×10⁶×75-bp reads per sample on a NextSeq 550 from the Stanford Functional Genomics Facility.

### RNA-Seq Analysis

For the RNA-Seq analysis, the sequences were aligned against the Mus musculus genome (mm9) using STAR (16). RSEM was used for calling transcripts and calculating transcripts per million (TPM) values as well as total counts (17). A counts matrix containing the number of counts for each gene and each sample was obtained. This matrix was analyzed by DESeq2 to calculate statistical analysis of significance of genes between samples (18). Up or downregulated genes, with p-value cutoff <0.05 were used for pathway analysis using DAVID (19). Heatmaps were generated on normalized counts and plotted on Z-score across rows using Seaborn data visualization library in python. The data reported in this paper have been deposited in the Gene Expression Omnibus (GEO) database GSE149924.

### Protein Extraction and Immunoblots

Total lysates were prepared using lysis buffer (50 mM Tris-HCl pH 7.5, 150 mM NaCl, 4 mM CaCl, 1.5% Triton X-100, protease inhibitors and micrococcal nuclease). For tissue extracts, lysates were homogenized a FastPrep 24 homogenizer (MP Biomedicals) for 40 seconds at a speed of 6 m/s. We used the following antibodies: 15-PGDH (Santa Cruz Biotechnology, cat # sc-271418); phospho-AKT (Ser 473) (Cell Signaling cat #4060), AKT (Cell signaling cat #2920); phospho-FoxO1 (Thr24)/FoxO3a (Thr32) Antibody (Cell Signaling cat #9464T); Foxo3a (Cell Signaling cat #2497); phospho-CREB (Ser133) (Cell Signaling cat #9198S); phospho-S6 ribosomal protein (Ser235/236) (Cell Signaling cat#4858); SMC1 (Bethyl Laboratories cat# A300-055A-T). We used HRP conjugated secondary antibodies and developed by incubating the membranes with ECL Western Blotting Substrate (Nacalai USA) and imaging using the ChemiDoc Imaging System (BioRad).

### 15-PGDH Kinetic Assay

### 15-PGDH Activity was Analyzed in Tissue Lysates Using the BioVision PicoProbe 15-PGDH Activity Assay Kit (Cat # K562) according to the protocol of the manufacturer

### Determination of PGE2 and Related Prostaglandins in Mouse Tissue by LC-MS/MS

### Analyte Standards

All prostaglandin standards-PGF2a; PGE2; PGD2; 15-keto PGE2; 13,14-dihydro 15-keto PGE2; PGD2-D4; PGA2; 13,14-dihydro 15-keto PGA2; PGE2-D4; and PGF2α-D9-were purchased from Cayman Chemical. For the PGE2-D4 and PGD2-D4 internal standards, positions 3 and 4 were labeled with a total of four deuterium atoms. For PGF2α-D9, positions 17, 18, 19 and 20 were labeled with a total of nine deuterium atoms.

### Calibration Curve Preparation

Analyte stock solutions (5 mg/mL) were prepared in DMSO. These stock solutions were serially diluted with acetonitrile/water (1:1 v/v) to obtain a series of standard working solutions, which were used to generate the calibration curve. Calibration curves were prepared by spiking 10 uL of each standard working solution into 200 μL of homogenization buffer (acetone/water 1:1 v/v; 0.005% BHT to prevent oxidation) followed by addition of 10 uL internal standard solution (3000 ng/mL each PGF2α-D9; PGD2-D4 and PGE2-D4). A calibration curve was prepared fresh with each set of samples. Calibration curve ranges: for PGA2; PGD2 and 13,14-dihydro 15-keto PGE2, 0.05 ng/mL to 500 ng/mL; for PGE2; 13,14-dihydro 15-keto PGA2 and PGF2u, 0.1 ng/mL to 500 ng/mL; and for 15-keto PGE2, 0.25 ng/mL to 500 ng/mL.

### Sample Preparation Procedure

The extraction procedure was modified from that of Prasain et al. (20) and included acetone protein precipitation followed by 2-step liquid-liquid extraction; the latter step enhances LC-MS/MS sensitivity. Butylated hydroxytoluene (BHT) and evaporation under nitrogen (N2) gas were used to prevent oxidation. Solid tissues were harvested, weighed, and snap-frozen with liquid nitrogen. Muscle tissue was combined with homogenization beads and 200 μL homogenization buffer in a polypropylene tube and processed in a FastPrep 24 homogenizer (MP Biomedicals) for 40 seconds at a speed of 6 m/s. After homogenization, 10 μL internal standard solution (3000 ng/mL) was added to tissue homogenate followed by shaking (Multi-Tube Vortexer, Thermo Scientific) for 2 minutes. Samples were centrifuged and the supernatant was transferred to a clean eppendorf tube. 200 μL hexane was added to the sample, followed by shaking for 15 minutes (Vortex Mixer, Thermo Scientific), then centrifugation. Samples were frozen at −80° C. for 40 minutes. The hexane layer was poured off from the frozen lower aqueous layer, and discarded. After thawing, 25 μL of 1N formic acid was added to the bottom aqueous layer, and the samples were vortexed. For the second extraction, 200 μL chloroform was added to the aqueous phase. Samples were shaken for 15 minutes to ensure full extraction. Centrifugation was performed to separate the layers. The lower chloroform layer was transferred to a new eppendorf tube and evaporated to dryness under nitrogen at 40° C. The dry residue was reconstituted in 100 μL acetonitrile/10 mM ammonium acetate (2:8 v/v) and analyzed by LC-MS/MS.

### LC-MS/MS

Since many prostaglandins are positional isomers with identical masses and have similar fragmentation patterns, chromatographic separation is critical. At least two SRM transitions—one quantifier and one qualifier—were carefully selected for each analyte. Distinctive qualifier to quantifier ion intensity ratios and retention times were essential to authenticate the target analytes. All analyses were carried out by negative electrospray LC-MS/MS using an LC-20AD_{XR} prominence liquid chromatograph and 8030 triple quadrupole mass spectrometer (Shimadzu). HPLC conditions: Acquity UPLC BEH C18 2.1×100 mm, 1.7 um particle size column was operated at 50° C. with a flow rate of 0.25 mL/min. Mobile phases consisted of A: 0.1% acetic acid in water and B: 0.1% acetic acid in acetonitrile. Elution profile: initial hold at 35% B for 5 minutes, followed by a gradient of 35%-40% in 3 minutes, then 40%-95% in 3 minutes; total run time was 14 minutes. Injection volume was 20 uL. Using these HPLC conditions, we achieved baseline separation of the analytes of interest. Selected reaction monitoring (SRM) was used for quantification. The mass transitions were as follows: PGD2: m/z 351.10→m/z 271.3 (quantifier); m/z 351.10→m/z 233.05 (qualifier) and m/z 351.10→m/z 189.15 (qualifier); PGE2: m/z 351.20→m/z 271.10 (quantifier); m/z 351.20→m/z 333.15 (qualifier) and m/z 351.20→m/z 315.20 (qualifier); PGF2a: m/z 353.10→m/z 3193.3 (quantifier) and m/z 353.10→m/z 309.20 25 (qualifier); 15 keto-PGE2: m/z 349.30→m/z 331.20 (quantifier) and m/z 349.30→m/z 113.00 (qualifier); 13, 14-dihydro 15-keto PGE2: m/z 351.20→m/z 333.30 (quantifier) and m/z 351.20→m/z 113.05 (qualifier); PGE2-D4: m/z 355.40→m/z 275.20 (quantifier); PGF2α-D9: m/z 362.20→m/z 318.30; PGD2-D4: m/z 355.10→m/z 275.40; PGA2: m/z 332.90→m/z 271.25 (quantifier) and m/z 332.90→m/z 189.10 (qualifier); and 13,14-dihydro 15-keto PGA2: m/z 332.90→m/z 235.15 (quantifier) m/z 332.90→m/z 113.00 (qualifier). Dwell time was 20-30 ms.

Quantitative data analysis was done using LabSolutions LCMS (Shimadzu). An internal standard method was used for quantification: PGE2-D4 was the internal standard for quantification of PGE2, 15-keto PGE2, and 13, 14-dihydro 15-keto PGE2, PGA2; 13,14-dihydro 15-keto PGA2. PGF2α-D9 was the internal standard for quantification of PGF2a; and PD2-D4 was the internal standard for quantification of PGD2. Calibration curves were linear (R>0.99) over the concentration range using a weighting factor of 1/X² where X is the concentration. The back—calculated standard concentrations were±15% from nominal values, and +20% at the lower limit of quantitation (LLOQ).

### In Vivo Muscle Force Measurement

The peak isometric torque (N·mm) of the ankle plantarflexors was assessed as previously described (21, 22). Briefly, the foot of anesthetized mice was placed on a footplate attached to a servomotor (model 300C-LR; Aurora Scientific). Two Pt-Ir electrode needles (Aurora Scientific) were inserted percutaneously and subcutaneously over the tibial nerve, just posterior/posterior-medial to the knee. The ankle joint was secured at a 90° angle. The peak isometric torque was achieved by varying the current delivered to the tibial nerve at a frequency of 200 Hz and a 0.1—ms square wave pulse. We performed three tetanic measurements on each muscle, with 1 min recovery between each measurement. Data were collected with the Aurora Scientific Dynamic Muscle Data Acquisition and Analysis Software.

### Statistical Analyses

A non-parametric Mann-Whitney test was used to determine the significance difference between untreated vs treated groups using α=0.05. ANOVA or multiple t-test was performed for multiple comparisons with significance level determined using Bonferroni correction or with Fisher's test as indicated in the figure legends. Unless otherwise described, data are shown as the mean+s.e.m.

### REFERENCES

1. D. J. Baker et al., Clearance of p16Ink4a-positive senescent cells delays ageing-associated disorders. Nature 479, 232-236 (2011).
2. D. J. Baker et al., Naturally occurring p16(Ink4a)-positive cells shorten healthy lifespan. Nature 530, 184-189 (2016).
3. Y. Zhang et al., TISSUE REGENERATION. Inhibition of the prostaglandin-degrading enzyme 15-PGDH potentiates tissue regeneration. Science 348, aaa2340 (2015).
4. C. Vinel et al., The exerkine apelin reverses age-associated sarcopenia. Nat Med 24, 1360-1371 (2018).
5. J. Chang et al., Clearance of senescent cells by ABT263 rejuvenates aged hematopoietic stem cells in mice. Nature Medicine 22, 78-83 (2016).
6. A. T. V. Ho et al., Prostaglandin E2 is essential for efficacious skeletal muscle stem-cell function, augmenting regeneration and strength. Proc Natl Acad Sci USA 114, 6675-6684 (2017).
7. B. D. Cosgrove et al., Rejuvenation of the muscle stem cell population restores strength to injured aged muscles. NatMed 20, 255-264 (2014).
8. A. Sacco, R. Doyonnas, P. Kraft, S. Vitorovic, H. M. Blau, Self-renewal and expansion of single transplanted muscle stem cells. Nature 456, 502-506 (2008).
9. P. M. Gilbert et al., Substrate elasticity regulates skeletal muscle stem cell self-renewal in culture. Science 329, 1078-1081 (2010).
10. G. Azzarello et al., Myosin isoform expression in rat rhabdomyosarcoma induced by Moloney murine sarcoma virus. J Cancer Res Clin Oncol 113, 417-429 (1987).
11. S. Schiaffino et al., Three myosin heavy chain isoforms in type 2 skeletal muscle fibres. JMuscle Res Cell Motil 10, 197-205 (1989).
12. L. R. Smith, E. R. Barton, SMASH—semi-automatic muscle analysis using segmentation of histology: a MATLAB application. SkeletMuscle 4, 21 (2014).
13. X. Xu et al., Human Satellite Cell Transplantation and Regeneration from Diverse Skeletal Muscles. Stem Cell Reports 5, 419-434 (2015).
14. P. M. Quiros, A. Goyal, P. Jha, J. Auwerx, Analysis of mtDNA/nDNA Ratio in Mice. Curr Protoc Mouse Biol 7, 47-54 (2017).
15. U. Raue et al., Transcriptome signature of resistance exercise adaptations: mixed muscle and fiber type specific profiles in young and old adults. JAppl Physiol (1985) 112, 1625-1636 (2012).
16. A. Dobin et al., STAR: ultrafast universal RNA-seq aligner. Bioinformatics 29, 15-21 (2013).
17. B. Li, C. N. Dewey, RSEM: accurate transcript quantification from RNA-Seq data with or without a reference genome. BMC Bioinformatics 12, 323 (2011).
18. M. I. Love, W. Huber, S. Anders, Moderated estimation of fold change and dispersion for RNA-seq data with DESeq2. Genome Biol 15, 550 (2014).
19. W. Huang da, B. T. Sherman, R. A. Lempicki, Systematic and integrative analysis of large gene lists using DAVID bioinformatics resources. Nat Protoc 4, 44-57 (2009).
20. J. K. Prasain, H. D. Hoang, J. W. Edmonds, M. A. Miller, Prostaglandin extraction and analysis in Caenorhabditis elegans. J Vis Exp, (2013).
21. E. L. Mintz, J. A. Passipieri, D. Y. Lovell, G. J. Christ, Applications of In Vivo Functional Testing of the Rat Tibialis Anterior for Evaluating Tissue Engineered Skeletal Muscle Repair. J Vis Exp, (2016).
22. K. A. Sheth et al., Muscle strength and size are associated with motor unit connectivity in aged mice. Neurobiol Aging 67, 128-136 (2018).

### Example 3. Targeting Prostaglandin E2 Degrading Enzyme to Amerliorate Non-Skeletal Muscle Tissue Function in Age-related Diseases and Conditions

As we age, quality of life is reduced and mortality is increased. Age-related diseases are a group of diseases that occur more frequently in people as they age which directly correlate to decreased longevity (1). These age-related diseases include cardiovascular diseases (atrial fibrillation, stroke, ischemic heart diseases, cardiomyopathies, endocarditis, intracerebral hemorrhage), chronic respiratory diseases (chronic obstructive pulmonary disease, asbestosis, silicosis), nutritional diseases (trachoma, diarrheal diseases, encephalitis), kidney diseases (chronic kidney diseases), gastrointestinal and digestive diseases (NASH, pancreatitis, ulcer, intestinal obstruction), neurological disorders (Alzheimer's, dementia, Parkinson's), sensory disorders (hearing loss, macular degeneration, glaucoma), skin and subcutaneous diseases (cellulitis, ulcer, fungal skin diseases, pyoderma), osteoporosis, osteoarthritis, rheumatoid arthritis and the like (2).

We determined previously that PGE2 stimulates muscle stem cells (MuSCs) to regenerate damaged muscles in young mice (3), in good agreement with findings regarding its function in regeneration in other tissues, including bone, colon, liver, and blood (4-6). We reasoned that PGE2 signaling might go awry in aging. Here we demonstrate a previously unrecognized role for the PGE2 degrading enzyme, 15-hydroxyprostaglandin dehydrogenase (15-PGDH), in aged tissues. Partial inhibition of 15-PGDH restores PGE2 and/or PGD2 to youthful levels, and can thereby rejuvenate tissue function. Our findings provide fresh insights into aging and uncover an innovative treatment strategy.

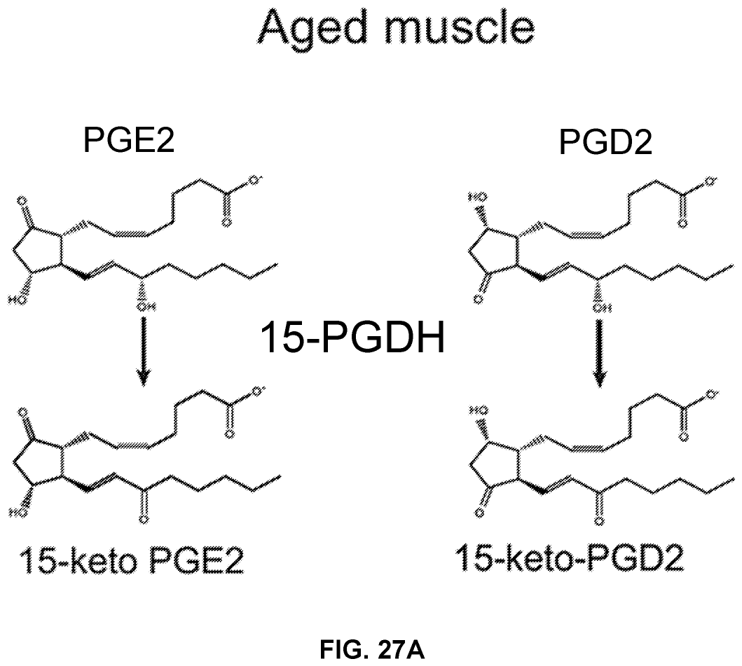

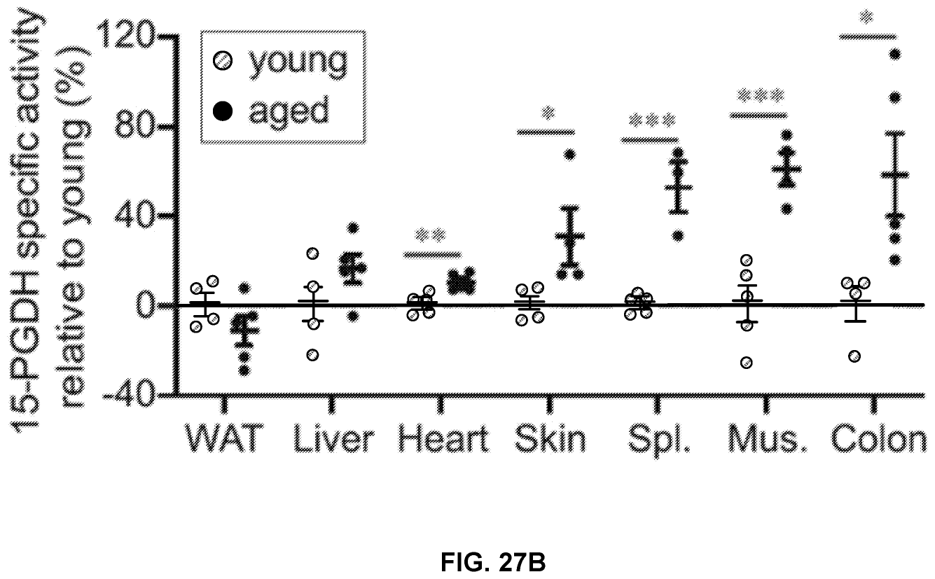

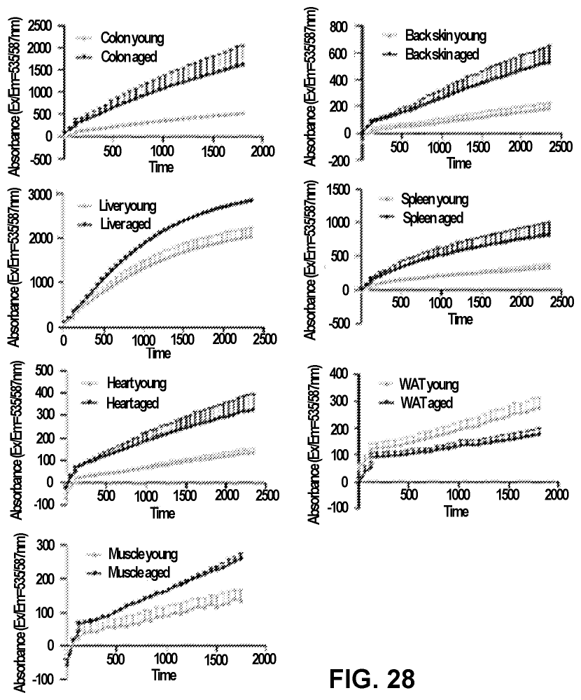

We hypothesized that a reduction in PGE2 was due to increased degradation by 15-PGDH in aged tissues (FIG. 27A). We found that the specific activity of the enzyme was indeed increased in aged tissues, including cardiac, skin, spleen and colon (FIGS. 27B and 28). Accordingly, inhibition of 15-PGDH can help ameliorate age-related diseases and conditions by restoring or increasing PGE2 and/or PGD2 levels in aged tissues.

We uncover 15-PGDH as a new marker of aging, detectable at elevated activity in numerous tissues such as heart, skin, colon, and spleen. Restoring PGE2 and/or PGD2 to youthful levels can therefore provide pleiotropic ameliorative effects, as 15-PGDH is upregulated in a range of tissues with aging.

### REFERENCES

1. D. S. Kehler, Age-related disease burden as a measure of population ageing. Lancet Public Health 4, e123-e124 (2019).
2. A. Y. Chang, V. F. Skirbekk, S. Tyrovolas, N. J. Kassebaum, J. L. Dieleman, Measuring population ageing: an analysis of the Global Burden of Disease Study 2017. Lancet Public Health 4, el59-el67 (2019).
3. A. T. V. Ho et al., Prostaglandin E2 is essential for efficacious skeletal muscle stem-cell function, augmenting regeneration and strength. Proc Natl Acad Sci USA 114, 6675-6684 (2017).
4. H. Chen et al., Prostaglandin E2 mediates sensory nerve regulation of bone homeostasis. Nat Commun 10, 181 (2019).
5. T. E. North et al., Prostaglandin E2 regulates vertebrate haematopoietic stem cell homeostasis. Nature 447, 1007-1011 (2007).
6. Y. Zhang et al., Inhibition of the prostaglandin-degrading enzyme 15-PGDH potentiates tissue regeneration. Science 348, aaa2340 (2015).

### Materials and Methods

### Mice

All experiments and protocols were performed in compliance with the institutional guidelines of Stanford University and Administrative Panel on Laboratory Animal Care (APLAC). Aged (>24 mo.) mice C57BL/6 were obtained from the US National Institute on Aging (NIA) for aged muscle studies, and young (2-4 mo.) wild-type C57BL/6 mice from Jackson Laboratory.

### 15-PGDH Kinetic Assay

15-PGDH activity was analyzed in tissue lysates using the BioVision PicoProbe 15-PGDH Activity Assay Kit (Cat # K562) according to the protocol of the manufacturer. Briefly, tissues were isolated and snap frozen in liquid nitrogen. Total lysates were prepared using lysis buffer (50 mM Tris-HCl pH 7.5, 150 mM NaCl, 4 mM CaCl, 1.5% Triton X-100, protease inhibitors and micrococcal nuclease) and homogenized using a FastPrep 24 homogenizer (MP Biomedicals) for 40 seconds at a speed of 6 m/s.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of enhancing a function of an aged skeletal muscle in a subject, the method comprising: administering to the aged skeletal muscle a 15-PGDH inhibitor in an amount effective to inhibit 15-PGDH activity and/or reduce 15-PGDH levels in one or more senescent cells in the aged skeletal muscle, thereby enhancing a function of the aged skeletal muscle.

2. A method of increasing muscle mass, muscle strength, and/or muscle endurance of an aged skeletal muscle in a subject, the method comprising: administering to the aged skeletal muscle a 15-PGDH inhibitor in an amount effective to inhibit 15-PGDH activity and/or reduce 15-PGDH levels in one or more senescent cells in the aged skeletal muscle, thereby increasing muscle mass, muscle strength, and/or muscle endurance of the aged skeletal muscle.

3. A method of increasing a level of PGE2 in an aged skeletal muscle in a subject, the method comprising: administering to the aged skeletal muscle a 15-PGDH inhibitor in an amount effective to increase PGE2 levels in the aged skeletal muscle, thereby increasing a level of PGE2 in the aged skeletal muscle.

4. The method of any one of claims 1-3, wherein the subject has one or more biomarkers of aging.

5. A method of rejuvenating an aged skeletal muscle in a subject having one or more biomarkers of aging, the method comprising: administering to the subject having one or more biomarkers of aging a 15-PGDH inhibitor in an amount effective to inhibit 15-PGDH activity and/or reduce 15-PGDH levels in the subject, thereby rejuvenating the aged skeletal muscle.

6. The method of claim 4 or 5, wherein the one or more biomarkers of aging is selected from the group consisting of: an increase in 15-PGDH levels relative to a level present in young skeletal muscle, a decrease in PGE2 levels relative to a level present in young skeletal muscle, an increase in a PGE2 metabolite relative to a level present in young skeletal muscle, an increase or a greater accumulation of senescent cells relative to a level present in young skeletal muscle, an increase in expression of one or more atrogenes relative to a level present in young skeletal muscle, a decrease in mitochondria biogenesis and/or function relative to a level present in young skeletal muscle, and an increase in transforming growth factor pathway signaling relative to a level present in young skeletal muscle.

7. The method of claim 6, wherein the one or more atrogenes is selected from the group consisting of: Atrogin1 (MAFbx1), MuSA (Fbxo30), and Trim63 (MuRF1).

8. The method of claim 6, wherein the increase in transforming growth factor pathway signaling comprises an increase in expression of one or more gene selected from the group consisting of: Activin receptor, Myostatin, a SMAD protein, and a bone morphogenetic protein.

9. The method of any one of claims 1-8, wherein the aged skeletal muscle has an increased accumulation of senescent cells relative to young skeletal muscle.

10. The method of any one of claims 1, 2, or 9, wherein the senescent cells express one or more senescent markers.

11. The method of any one of claims 1, 2, 9, or 10, wherein the senescent cells have an increased level of one or more senescent markers relative to non-senescent cells.

12. The method of claim 10 or 11, wherein the one or more senescent markers is selected from the group consisting of: p15Ink4b, p16Ink4a, p19Arf, p21, Mmp13, I11a, I11b, and 116.

13. The method of any one of claims 1, 2, or 19-12, wherein the senescent cells are macrophages.

14. The method of any one of claims 1-13, wherein the aged skeletal muscle is uninjured and/or has not undergone exercise and/or has not undergone regeneration.

15. The method of any one of claims 1-14, further comprising administering a senolytic agent to the aged skeletal muscle.

16. The method of claim 15, wherein the senolytic agent is selected from the group consisting of: a Bc12 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof.

17. The method of any one of claims 1-16, wherein the 15-PGDH inhibitor is selected from the group consisting of: a small molecule compound, a blocking antibody, a nanobody, and a peptide.

18. The method of any one of claims 1-17, wherein the 15-PGDH inhibitor is SW033291.

19. The method of any one of claims 1-16, wherein the 15-PGDH inhibitor is selected from the group consisting of: an antisense oligonucleotide, microRNA, siRNA, and shRNA.

20. The method of any one of claims 1-19, wherein the subject is a human.

21. The method of any one of claims 1-20, wherein the subject is at least 30 years of age.

22. The method of any one of claims 1-21, wherein the administering comprises systemic administration or local administration.

23. The method of any one of claims 1-22, wherein a level of PGE2 is increased in the aged skeletal muscle relative to a level of PGE2 present in the aged skeletal muscle prior to the administering of the 15-PGDH inhibitor.

24. The method of any one of claims 1-23, wherein a level of PGE2 is increased by at least 10% relative to a level of PGE2 present in the aged skeletal muscle prior to the administering of the 15-PGDH inhibitor.

25. The method of any one of claims 1-24, wherein a level of PGE2 is increased to a level that is substantially similar to a level present in young skeletal muscle.

26. The method of any one of claims 1-25, wherein a level of PGE2 is increased to a level that is within about 50% or less of a level present in young skeletal muscle.

27. The method of any one of claims 1-26, wherein the method results in an increase in myofiber and/or myotube cross-sectional area and/or diameter.

28. The method of any one of claims 1-27, wherein the method results in an increase in cross-sectional area and/or diameter of oxidative (type IIa) and/or glycolytic (type IIb) fibers.

29. The method of any one of claims 1-28, wherein the 15-PGDH inhibitor reduces or blocks 15-PGDH expression.

30. The method of any one of claims 1-29, wherein the 15-PGDH inhibitor reduces or blocks enzymatic activity of 15-PGDH.

31. The method of any one of claims 1-30, wherein the method results in an increase in muscle mass, an increase in muscle strength, an increase in muscle endurance, or any combination thereof of the aged skeletal muscle.

32. The method of any one of claims 1-31, wherein the method results in an increase in muscle mass, an increase in muscle strength, an increase in muscle endurance, or any combination thereof of the aged skeletal muscle relative to the aged skeletal muscle prior to the administering of the 15-PGDH inhibitor.

33. The method of any one of claims 1-32, wherein the method results in an increase in muscle mass, an increase in muscle strength, an increase in muscle endurance, or any combination thereof of the aged skeletal muscle to a level substantially similar to a level present in young skeletal muscle.

34. The method of any one of claims 1-33, wherein the method results in an increase in muscle mass, an increase in muscle strength, an increase in muscle endurance, or any combination thereof of the aged skeletal muscle to a level within about 50% or less of a level present in young skeletal muscle.

35. The method of any one of claims 1-34, wherein the method results in an enhanced function of the aged skeletal muscle.

36. The method of any one of claims 1-35, wherein the method results in an enhanced function of the aged skeletal muscle relative to the aged skeletal muscle prior to the administering of the 15-PGDH inhibitor.

37. The method of any one of claims 1-36, wherein the method results in an enhanced function of the aged skeletal muscle to a level substantially similar to a level present in young skeletal muscle.

38. The method of any one of claims 1-37, wherein the method results in an enhanced function of the aged skeletal muscle to a level within about 50% or less of a level present in young skeletal muscle.

39. The method of any one of claims 35-38, wherein the function is an increase in protein synthesis, an increase in cell proliferation, an increase in cell survival, a decrease in protein degradation, or any combination thereof.

40. The method of any one of claims 1-39, wherein the method results in decreased levels of a PGE2 metabolite in the aged skeletal muscle relative to the aged skeletal muscle prior to the administering of the 15-PGDH inhibitor, and/or to a level substantially similar to a level present in young skeletal muscle.

41. The method of claim 40, wherein the PGE2 metabolite is selected from the group consisting of: 15-keto PGE2 and 13,14-dihydro-15-keto PGE2.

42. The method of any one of claims 1-41, wherein the subject has sarcopenia due to aging.

43. The method of any one of claims 1-42, wherein an expression level of one or more atrogenes is decreased relative to the aged skeletal muscle prior to the administering of the 15-PGDH inhibitor and/or to a level substantially similar to a level present in young skeletal muscle.

44. The method of any one of claims 1-43, wherein an expression level of one or more components of a mitochondria complex is increased relative to the aged skeletal muscle prior to the administering of the 15-PGDH inhibitor and/or to a level substantially similar to a level present in young skeletal muscle.

45. The method of claim 44, wherein the one or more components of a mitochondria complex is selected from the group consisting of: Ndufa11, Ndufa12, Ndufa13, Ndufa2, Ndufa3, Ndufa4, Ndufa5, Ndufa10, Ndufb5, Ndufcl, Ndufs4, Ndufs8, Ndufv1, Ndufv2, Uqcrb, Uqcrcl, Uqcrh, Uqcrq, Ucqr10, Cox8b, Cox7al, Cox7a2, Cox7b, Cox6c, Cox5a, Cox5b, Atp5fl, Atp5gl, Atp5h, Atp5j2, Atp5o, Atp5e, and Atp5k.

46. The method of any one of claims 1-45, wherein an expression level of peroxisome proliferator-activated receptor gamma coactivator 1-alpha (Pgc1α) is increased relative to the aged skeletal muscle prior to the administering of the 15-PGDH inhibitor and/or to a level substantially similar to a level present in young skeletal muscle.

47. The method of any one of claims 1-46, wherein an expression level of one or more genes selected from the group consisting of: Tnfaipl, Klhdc8a, Fbxwll, Tnfaip3, Herc3, Herc2, Hdac4, Traf6, Ankibl, Mib1, Pja2, Ubr3, Thbsl, Smad3, Acvr2a, Rgmb, Tgfb2, and Mstn is decreased relative to the aged skeletal muscle prior to the administering of the 15-PGDH inhibitor and/or to a level substantially similar to a level present in young skeletal muscle.

48. The method of any one of claims 1-47, wherein the method is independent of an increase in proliferation of muscle stem cells (MuSCs) in the subject.

49. The method of any one of claims 1-48, wherein the administering comprises once a day, twice a day, once a week, or once a month administration.

50. A method of rejuvenating an aged non-skeletal muscle tissue in a subject, the method comprising: administering to the subject an amount of a 15-PGDH inhibitor effective to inhibit 15-PGDH activity and/or reduce 15-PGDH levels in the subject, thereby rejuvenating the aged non-skeletal muscle tissue.

51. The method of claim 50, wherein the administering increases a level of PGE2 in the aged non-skeletal muscle tissue of the subject.

52. The method of claim 50 or 51, wherein a level of PGE2 in the aged non-skeletal muscle tissue is increased relative to the aged non-skeletal muscle tissue prior to the administering of the 15-PGDH inhibitor.

53. The method of any one of claims 50-52, wherein a level of PGE2 in the aged non-skeletal muscle tissue is increased by at least 10% relative to the aged non-skeletal muscle tissue prior to the administering of the 15-PGDH inhibitor.

54. The method of any one of claims 50-53, wherein a level of PGE2 in the aged non-skeletal muscle tissue is increased to a level substantially similar to a level present in young non-skeletal muscle tissue.

55. The method of any one of claims 50-54, wherein a level of PGE2 in the aged non-skeletal muscle tissue is increased to a level within about 50% or less of a level present in young non-skeletal muscle tissue.

56. The method of any one of claims 50-55, wherein the aged non-skeletal muscle tissue is selected from the group consisting of: epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof.

57. The method of any one of claims 50-56, wherein the subject has one or more biomarkers of aging.

58. The method of claim 57, wherein the one or more biomarkers of aging is selected from the group consisting of: an increase in 15-PGDH levels relative to young non-skeletal muscle tissue, a decrease in PGE2 levels relative to young non-skeletal muscle tissue, an increase in a PGE2 metabolite relative to young non-skeletal muscle tissue, an increase or a greater accumulation of senescent cells relative to young non-skeletal muscle tissue, an increase in expression of one or more atrogenes relative to young non-skeletal muscle tissue, a decrease in mitochondria biogenesis and/or function relative to young non-skeletal muscle tissue, and an increase in transforming growth factor pathway signaling relative to young non-skeletal muscle tissue.

59. The method of any one of claims 50-58, wherein the aged non-skeletal muscle tissue has an increased accumulation of senescent cells relative to young non-skeletal muscle tissue.

60. The method of claim 58 or 59, wherein the senescent cells express one or more senescent markers.

61. The method of any one of claims 58-60, wherein the senescent cells have an increased level of one or more senescent markers relative to non-senescent cells.

62. The method of claim 60 or 61, wherein the one or more senescent markers is selected from the group consisting of: p15Ink4b, p16Ink4a, p19Arf, p21, Mmp13, I11a, I11b, and 116.

63. The method of any one of claims 60-62, wherein the senescent cells are macrophages.

64. The method of any one of claims 50-63, further comprising administering a senolytic agent to the aged non-skeletal muscle tissue.

65. The method of claim 64, wherein the senolytic agent is selected from the group consisting of: a Bc12 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof.

66. The method of any one of claims 50-65, wherein the 15-PGDH inhibitor is selected from the group consisting of: a small molecule compound, a blocking antibody, a nanobody, and a peptide.

67. The method of any one of claims 50-66, wherein the 15-PGDH inhibitor is SW033291.

68. The method of any one of claims 50-65, wherein the 15-PGDH inhibitor is selected from the group consisting of: an antisense oligonucleotide, microRNA, siRNA, and shRNA.

69. The method of any one of claims 50-68, wherein the subject is a human.

70. The method of any one of claims 50-69, wherein the subject is at least 30 years of age.

71. The method of any one of claims 50-70, wherein the 15-PGDH inhibitor reduces or blocks 15-PGDH expression.

72. The method of any one of claims 50-71, wherein the 15-PGDH inhibitor reduces or blocks enzymatic activity of 15-PGDH.

73. The method of any one of claims 50-72, wherein a function of the aged non-skeletal muscle is enhanced relative to a function of the aged non-skeletal muscle prior to the administering of the 15-PGDH inhibitor.

74. The method of any one of claims 50-73, wherein a function of the aged non-skeletal muscle tissue is enhanced by at least 10% relative to the function of the aged non-skeletal muscle prior to the administering of the 15-PGDH inhibitor.

75. The method of any one of claims 50-74, wherein a function of the aged non-skeletal muscle tissue is enhanced to a level that is substantially similar to a level present in young non-skeletal muscle tissue.

76. The method of any one of claims 50-75, wherein a function of the aged non-skeletal muscle tissue is enhanced to a level that is within about 50% or less of a level present in young non-skeletal muscle tissue.

77. The method of any one of claims 73-76, wherein the function comprises increased protein synthesis, increased cell proliferation, increased cell survival, decreased protein degradation, or any combination thereof.

78. The method of any one of claims 50-77, wherein the method results in decreased levels of a PGE2 metabolite in the aged non-skeletal muscle tissue relative to the aged non-skeletal muscle tissue prior to the administering of the 15-PGDH inhibitor and/or to a level that is substantially similar to a level present in young non-skeletal muscle.

79. The method of claim 78, wherein the PGE2 metabolite is selected from the group consisting of: 15-keto PGE2 and 13,14-dihydro-15-keto PGE2.

80. A method of enhancing a function of a skeletal muscle in a subject, the method comprising: administering to the subject a 15-PGDH inhibitor in an amount effective to inhibit 15-PGDH activity and/or reduce 15-PGDH levels in the skeletal muscle, thereby enhancing a function of the skeletal muscle in the subject,
wherein the skeletal muscle is healthy, and
wherein the method is independent of an increase in proliferation of muscle stem cells (MuSCs) in the subject.

81. The method of claim 80, wherein the skeletal muscle is uninjured.

82. The method of claim 80 or 81, wherein the skeletal muscle is not undergoing regeneration.

83. The method of any one of claims 80-82, wherein the skeletal muscle has not undergone significant or substantial exercise.

84. The method of any one of claims 80-83, wherein the function is enhanced relative to the skeletal muscle prior to the administering of the 15-PGDH inhibitor.

85. The method of any one of claims 80-84, wherein the function is an increase in protein synthesis, an increase in cell proliferation, an increase in cell survival, a decrease in protein degradation, or any combination thereof.

86. The method of any one of claims 80-85, wherein the method results in an increase in muscle mass, an increase in muscle strength, an increase in muscle endurance, or any combination thereof relative to the skeletal muscle prior to the administering of the 15-PGDH inhibitor.

87. The method of any one of claims 80-86, wherein the skeletal muscle is young skeletal muscle.

88. The method of claim 87, wherein the subject is less than 30 years of age.

89. The method of any one of claims 80-86, wherein the skeletal muscle is aged skeletal muscle.

90. The method of claim 89, wherein the subject is greater than 30 years of age.
